(19) United States
(12) Patent Application Publication
(10) Pub. No.: US 2008/0038809 A1
(43) Pub. Date: Feb. 14, 2008

(54) ISOLATED LECTIN POLYPEPTIDES CONSISTING OF TRUNCATED MAMMALIAN UDP-GalNAc:POLYPEPTIDE N- ACETYLGALACTOSAMINYLTRANSFERASES

(75) Inventors: Henrik CLAUSEN, Holte (DK); Eric P. BENNETT, Lyngby (DK)

Correspondence Address: DARBY & DARBY P.C., P.O. BOX 770, Church Street Station, New York, NY, 10008-0770, US

(73) Assignee: GLYCOZYM APS, Horsholm (DK)

(21) Appl. No.: 11/870,966

(22) Filed: Oct. 11, 2007

Related U.S. Application Data
(63) Continuation of application No. 10/705,401, filed on Nov. 10, 2003, now abandoned.
(63) Continuation of application No. PCT/DK03/00763, filed on Nov. 7, 2003.
(60) Provisional application No. 60/425,204, filed on Nov. 8, 2002.

(51) Int. Cl. C07K 14/42; C12N 9/10

(52) U.S. Cl. 435/193; 530/396

(57) ABSTRACT

Novel methods for identification of inhibitors or modulators of binding activities mediated by lectin domains of polypeptide GalNAc-transferases are disclosed. Direct binding activity of GalNAc-transferase lectins has been demonstrated for the first time and methods to measure lectin mediated binding of isolated lectins or enzymes with lectin domains are disclosed. The present invention specifically discloses a novel selective inhibitor of polypeptide GalNAc-transferase lectin domains, which provides a major advancement in that this inhibitor and related inhibitors sharing common characteristics of activity bind lectin domains without serving as acceptor substrate for glycosyltransferases involved in synthesis of O-glycans. This inhibitor is represented by the O-anomeric configuration of GalNAc-benzyl, GalNAcβ-benzyl. Methods for inhibiting intracellular transport, cell surface expression, and secretion of mucins and O-glycosylated glycoproteins without affecting O-glycosylation processing are disclosed using the novel selective inhibitor identified.

This application claims priority from U.S. Provisional Application Ser. No. 60/425,204, filed Nov. 8, 2002, and from International Application PCT/DK03/______ (Attorney docket number 04305/200H154-WO1), filed Nov. 6, 2003, each of which is hereby incorporated by reference in its entirety.

### TECHNICAL FIELD

The present invention relates generally to the biosynthesis, sorting and secretion of mucins, O-glycosylated glycoproteins, and glycoproteins. More specifically, it relates to modulation of the functions of a homologous family of UDP-N-acetyl-α-D-galactosamine:polypeptide N-acetylgalactosaminyltransferases (GalNAc-transferases), which are generally characterized by the enzyme activity to add N-acetylgalactosamine (GalNAc) to the hydroxy group of serine and threonine amino acid residues in peptides, glycopeptides, proteins, and glycoproteins.

In particular, this invention concerns a method of inhibiting or modulating functions mediated by lectin domains of polypeptide GalNAc-transferases. More particularly, this invention is related to a lectin domain found in the C-terminal region of most GalNAc-transferases, which is structurally and functionally distinct from the catalytic domain of the enzymes and can be expressed in a functional form as a truncated or mutated protein that is enzymatically inactive. Still more particularly, this invention is related to the lectin domains of more than 16 GalNAc-transferases, designated GalNAc-T1 to GalNAc-T16.

Further, this invention concerns a method of screening one or more test substances for the ability to inhibit or modulate polypeptide GalNAc-transferase lectins in a cell-free or cell-based assay, in order to find agents which are effective in binding to one or more lectin domains of polypeptide GalNAc-transferases, for example, selective inhibitors of the binding properties of the above mentioned lectin domains and selective inhibitors of the effects that these lectin domains exert on intracellular transport, transport to cell surface, and secretion of mucins, glycoproteins, and proteins.

Even further, this invention provides a preferable inhibitor, GalNAcβ-benzyl, which is a novel inhibitor and representative of a novel group of inhibitors which display the common characteristic of selectively inhibiting lectins of polypeptide GalNAc-transferases in direct binding assays and not serve as substrates for other glycosyltransferases involved in O-glycan biosyntheses, while exhibiting inhibitory activity of secretion and intracellular transport of mucins and glycoproteins in cells. GalNAcβ-benzyl and related inhibitors with the same biological functions represent preferable selective inhibitor compared to GalNAcα-benzyl because these do not serve as substrates for glycosyltransferases extending O-glycans and do not provide a general inhibition of the O-glycosylation process in cells.

### BACKGROUND OF THE INVENTION

Mucin-type O-glycosylation, one of the most abundant forms of protein glycosylation, is found on secreted and cell surface associated glycoproteins of all eukaryotic cells except yeast. Mucin-type O-glycans contribute to a number of important molecular functions, including: direct effects on protein conformation, solubility, and stability; specific receptor functions that regulate cell trafficking and cell-cell interactions; and microbial clearance. Mucin-type O-glycans are synthesised in the Golgi through the sequential addition of saccharide residues, first to hydroxyl groups on serines and threonines of a protein core and subsequently to hydroxyl groups on the growing saccharide chains that extend from the protein core. There is great diversity in the structures created by O-glycosylation (hundreds of potential structures), which are produced by the catalytic activity of hundreds of glycosyltransferase enzymes that are resident in the Golgi complex. Diversity exists at the level of the glycan structure and in positions of attachment of O-glycans to protein backbones. Despite the high degree of potential diversity, it is clear that O-glycosylation is a highly regulated process that shows a high degree of conservation among multicellular organisms.

The factors that regulate the attachment of O-glycans to particular protein sites and their extension into specific structures are poorly understood. Longstanding hypotheses in this area propose that mucin-type O-glycosylation occurs in a stochastic manner where structure of acceptor proteins combined with topology and kinetic properties of resident Golgi glycosyltransferases determine the order and degree of glycosylation¹. This concept does not fully explain the high degree of regulation and specialisation that governs the O-glycosylation process. In particular it is difficult to envision how large mucin molecules with high densities of O-glycans are glycosylated in the Golgi by stochastic mechanisms that also create other sparsely glycosylated proteins.

The first step in mucin-type O-glycosylation is catalysed by one or more members of a large family of UDP-GalNAc: polypeptide N-acetylgalactosaninyltransferases (GalNAc-transferases) (EC 2.4.1.41), which transfer GalNAc to serine and threonine acceptor sites². To date twelve members of the mammalian GalNAc-transferase family have been identified and characterized³, and several additional putative members of this gene family have been predicted from analysis of genome databases. The GalNAc-transferase isoforms have different kinetic properties and show differential expression patterns temporally and spatially, suggesting that they have distinct biological functions 2. Sequence analysis of GalNAc-transferases have led to the hypothesis that these enzymes contain two distinct subunits: a central catalytic unit, and a C-terminal unit with sequence similarity to the plant lectin ricin, designated the “lectin domain”⁴⁻⁷. Previous experiments involving site-specific mutagenesis of selected conserved residues confirmed that mutations in the catalytic domain eliminated catalytic activity. In contrast, mutations in the “lectin domain” had no significant effects on catalytic activity of the GalNAc-transferase isoform, GalNAc-T1⁴. Thus, the C-terminal “lectin domain” was believed not to be functional and not to play roles for the enzymatic functions of GalNAc-transferases⁴.

However, recent evidence demonstrates that some GalNAc-transferases exhibit unique activities with partially GalNAc-glycosylated glycopeptides. The catalytic actions of at least three GalNAc-transferase isoforms, GalNAc-T4, -T7, and -T10, selectively act on glycopeptides corresponding to mucin tandem repeat domains where only some of the clustered potential glycosylation sites have been GalNAc glycosylated by other GalNAc-transferases⁸⁻¹¹. GalNAc-T4 and -T7 recognize different GalNAc-glycosylated peptides and catalyse transfer of GalNAc to acceptor substrate sites in addition to those that were previously utilized. One of the functions of such GalNAc-transferase activities is predicted to represent a control step of the density of O-glycan occupancy in mucins and mucin-like glycoproteins with high density of O-glycosylation. It was hypothesized that such sequential actions of multiple GalNAc-transferase isoforms may be required to complete O-glycan attachments to some mucin peptide sequences allowing for detailed control of density.

One example of this is the glycosylation of the cancer-associated mucin MUC1. MUC1 contains a tandem repeat O-glycosylated region of 20 residues (HGVTSAPDTRPAPGSTAPPA) with five potential O-glycosylation sites. GalNAc-T1, -T2, and -T3 can initiate glycosylation of the MUC1 tandem repeat and incorporate at only three sites (HGVtSAPDTRPAPGstAPPA, GalNAc attachment sites in lower-case). GalNAc-T4 is unique in that it is the only GalNAc-transferase isoform identified so far that can complete the O-glycan attachment to all five acceptor sites in the 20 amino acid tandem repeat sequence of the breast cancer associated mucin, MUC1. GalNAc-T4 transfers GalNAc to at least two sites not used by other GalNAc-transferase isoforms on the GalNAc₄TAP24 glycopeptide (tAPPAHGVtSAPDTRPAPGstAPP, GalNAc attachment sites in lower case)¹⁰. An activity such as that exhibited by GalNAc-T4 appears to be required for production of the glycoform of MUC1 expressed by cancer cells where all potential sites are glycosylated¹². Normal MUC1 from lactating mammary glands has approximately 2.6 O-glycans per repeat¹³ and MUC1 derived from the cancer cell line T47D has 4.8 O-glycans per repeat¹². The cancer-associated form of MUC1 is therefore associated with higher density of O-glycan occupancy and this is accomplished by a GalNAc-transferase activity identical to or similar to that of GalNAc-T4.

The specific mechanism by which GalNAc-T4, -T7, and -T10 recognize and function with GalNAc-glycosylated glycopeptides is not known. However, it was originally demonstrated that the GalNAc-glycopeptide specificity exerted by GalNAc-T4 is directed or at least dependent on its lectin domain. A single amino acid substitution in the T4 lectin domain predicted to inactivate its function abolished the GalNAc-glycopeptide specificity of T4 without adversely affecting the basic catalytic mechanism of the transferase². This suggests that the lectin domain interacts with GalNAc-glycopeptides and confers a novel catalytic function to the enzyme protein. Despite extensive attempts it has in the past not been possible to demonstrate actual binding of the transferase and lectin to sugars and glycopeptides, but it was possible to demonstrate selective inhibition of the GalNAc-glycopeptide activity of GalNAc-T4 using 230 mM concentration of GalNAc². The present inventors and coworkers demonstrated that mM concentrations of GalNAcα-benzyl can inhibit the lectin mediated GalNAc-glycopeptide substrate specificity of GalNAc-T4 as well as -T7 (PCT WO 01/85215 A2). Polypeptide GalNAc-transferases, which have not displayed apparent GalNAc-glycopeptide specificities, also appear to be modulated by their putative lectin domains (PCT WO 01/85215 A2). Recently, it was found that mutations in the GalNAc-T1 putative lectin domain, similarly to those previously analysed in GalNAc-T4², modified the activity of the enzyme in a similar fashion as GalNAc-T4. Thus, while wild type GalNAc-T1 added multiple consequtive GalNAc residues to a peptide substrate with multiple acceptor sites, mutated GalNAc-T1 failed to add more than one GalNAc residue to the same substrate¹⁴. The mechanism is however not understood.

Glycosylation confers physico-chemical properties including protease resistance, solubility, and stability to proteins¹⁵⁻¹⁷. Glycosylation furthermore confers changes in immunological responses to proteins and glycoproteins. O-glycosylation on mucins and mucin-like glycoproteins protect these molecules found in the extracellular space and body fluids from degradation. Control of O-glycosylation with respect to sites and number (density) of O-glycan attachments to proteins as well as control of the O-glycan structures made at specific sites or in general on glycoproteins, is of interest for several purposes. Diseased cells e.g. cancer cells often dramatically change their O-glycosylation and the altered glycans and glycoproteins may constitute targets for therapeutic and diagnostic measures^{18,19}. Mucins functioning in body fluids may have different properties depending on density and structure of O-glycans attached in protection against disease, including infections by microorganisms. Furthermore, mucins with different glycosylation may change physico-chemical properties including stability and solubility properties that may influence turnover and removal of mucous. A number of lung diseases, e.g. cystic fibrosis, asthma, chronic bronchitis, smokers lungs, are associated with symptomatic mucous accumulation²⁰⁻²², and it is likely that the nature and structure of mucins play a role in the pathogenesis of such diseases.

Partial inhibitors of O-glycosylation in cells have been reported. Aryl-N-acetyl-α-galactosaminides such as benzyl-, phenyl-, and p-nitrophenyl-GalNAc were originally found to inhibit the second step in O-glycosylation, the O-glycan processing step, by inhibiting synthesis of core 1 (Galβ1-3GalNAcα1-R) and more complex structures²³. Benzyl-αGalNAc was also found to inhibit sialylation. It is generally believed that the downstream effects of benzyl-αGalNAc treatment are mediated by substrate competition of biosynthetic glycosylation products of benzyl-αGalNAc. Thus, e.g. the immediate glycosylation product of benzyl-αGalNAc is Galβ1-3GalNAcα-benzyl and this serves as an efficient substrate for the core 1 α2-3sialyltransferase ST3Gal-I^{24,25}. GalNAcα-benzyl has been the most widely used inhibitor of O-glycosylation, but it has only been used in cell culture as effective treatment concentrations lead to intracellular build-up of vesicles with GalNAcα-benzyl products and treated cells change morphology and growth characteristics²⁶.

Treatment of cells with benzyl-αGalNAc inhibit O-glycan processing and affect apical sorting of some O-glycosylated proteins²⁷⁻²⁹. The mechanism for this is generally believed to be through inhibition of sialylation²⁶. Inhibition of mucin secretion has also been observed in culture cells, more specifically HT29 MTX cells, but this effect is not generally found in mucin secreting cells²⁶.

True inhibitors of O-glycosylation, i.e. inhibitors of the initial O-glycan attachment process governed by polypeptide GalNAc-transferases have not been identified. Benzyl-αGalNAc has been shown to exhibit inhibitory effect on the GalNAc-glycopeptide substrate specificity of some polypeptide GalNAc-transferases in test tube enzyme assays (PCT WO 01/85215 A2), however the effect of this compound in vivo on density of O-glycosylation is unknown.

Inhibitors of the initiating step in O-glycosylation could completely or selectively block attachment of O-glycans to O-glycosylation sites in proteins. Compounds inhibiting the catalytic function of a selected subset of the polypeptide GalNAc-transferase family may be predicted to only lead to partial inhibition of O-glycosylation capacity of cells. Proteins with no or little O-glycosylation may have entirely different biological properties than their normal glycosylated counterparts. Complete inhibition of O-glycosylation is not desirable because of the many diverse functions of O-glycans, and it is expected to result in cell death. Selective inhibition of O-glycosylation on the other hand is desirable in many cases such as cancer cells producing glycoproteins and mucins with denser O-glycosylation than normal cells. For example breast cancer cells appear to hyperglycosylate the cancer-associated cell surface mucin MUC1 compared to glycosylation in normal cells^{12,30}. The overexpression of MUC1 and hyperglycosylation found in cancer cells are likely to be important for the pathobiology of cancers. Methods of inhibiting the hyperglycosylation of mucins in cancer cells are desirable.

It is apparent from the above that inhibitors in the prior art interfere with O-glycan processing, i.e. the glycosylation process that extend GalNAc residues directly attached to proteins at serine and threonine residues. Existing inhibitors of O-glycosylation are not suitable for therapeutic treatment in mammals including man as they profoundly affect O-glycosylation processing as well as lead to undesired morphological and growth effects on culture cells.

Consequently, there exists a need in the art for therapeutic compounds that display selectively and limited inhibition of O-glycosylation without generally affecting the process of O-glycosylation. The present invention meets these needs, and further presents other related advantages.

### SUMMARY OF THE INVENTION

The present invention provides for isolated polypeptide lectin domains of GalNAc-transferases. The present invention also provides for mammalian GalNAc-transferase lectin domains, lectin-functional variants and fragments thereof. The present invention also provides for human GalNAc-T1-T16 lectin domains having the sequences disclosed in Table III herein.

The present invention also provides for isolated nucleic acids encoding lectin domains of GalNAc-transferases. These nucleic acid sequences can encode for mammalian GalNAc-transferase lectin domains, lectin-functional variants and fragments thereof. The present invention also provides for isolated nucleic acids encoding human GalNAc-T1-T16 (these sequences are disclosed in Table III, herein).

The present invention further provides a method of inhibiting or modulating specific functions mediated by lectin domains of polypeptide GalNAc-transferases comprising administering an effective amount of an appropriate agent which is effective in binding to one or more lectin domains of polypeptide GalNAc-transferases and inhibiting functions mediated by said lectin domains.

The present invention specifically discloses a novel selective inhibitor of polypeptide GalNAc-transferase lectin domains, which provides a major advancement in that this inhibitor and related inhibitors sharing common characteristics of activity binds lectin domains without serving as acceptor substrate for glycosyltransferases involved in synthesis of O-glycans. For this reason treatment of cells with this and related compounds is not expected to affect O-glycosylation extension including O-glycan branching and sialylation. A preferred embodiment of this invention is a selective inhibitor of GalNAc-transferase lectins that does not serve as substrates for other glycosyltransferases.

In a preferred embodiment, the novel selective GalNAc-transferase lectin inhibitor is represented by the β-anomeric configuration of GalNAc-benzyl, GalNAcβ-benzyl. Additional preferred selective inhibitors including but not limited to the following: GalNAcβ1-R (R represents any aglycone such as benzyl, phenyl, p-nitrophenyl, or umbrelliferyl, without limitation), selective GalNAcβ-peptides, mimetics or compounds derived herefrom. The identifying characteristics of the preferred selective inhibitor is that it retains the binding activity for lectin domains similar to GalNAcβ-benzyl and GalNAcα-benzyl as determined in binding assays as disclosed in the present invention, and lacks the ability to serve as acceptor substrate for glycosyltransferases involved in O-glycosylation as determined by standard enzyme assays widely known by the skilled in the art.

The lectin domains confer unique properties to the GalNAc-transferases including but not limited to selective GalNAc-glycopeptide substrate specificity, as well as binding properties for peptides and carbohydrates to enhance catalytic properties and other functions related to the O-glycosylation process. In a preferred embodiment, methods of selectively blocking the lectin mediated acceptor substrate specificities of such GalNAc-transferase isoforms by βGalNAc and βGalNAc containing structures are disclosed.

The present invention provides a novel method for large scale screening of test substances for the ability to inhibit lectin-mediated activity of polypeptide GalNAc-transferases in a cell-free assay, which comprises:
(i) contacting an isolated polypeptide GalNAc-transferase, an isolated lectin domain from a polypeptide GalNAc-transferase, or a fragment of a polypeptide GalNAc-transferase capable of displaying lectin-mediated binding on its substrate, with one or more test substances under assay conditions suitable for the detection of said binding ability; and
(ii) measuring whether said lectin-mediated activity is thereby inhibited or modulated by one or more of the substances.

The present invention also provides a method of screening one or more test substances for the ability to inhibit or modulate intracellular transport and/or cell surface expression of mucins, O-glycosylated glycoproteins, glycoproteins and proteins in a cell-based assay, which comprises:
(i) contacting a cell that expresses mucins, O-glycosylated glycoproteins, glycoproteins and proteins, with one or more test substances under assay conditions suitable for the detection of inhibition or modulation of said expression; and
(ii) measuring whether intracellular transport and cell surface expression of said mucins, O-glycosylated glycoproteins, glycoproteins and proteins are thereby inhibited or modulated by one or more of the substances.

The present invention also provides a method of screening one or more test substances for the ability to inhibit or modulate secretions of mucins, O-glycosylated glycoproteins, glycoproteins and proteins in a cell-based assay, which comprises:
(i) contacting a cell that secretes mucins, O-glycosylated glycoproteins, glycoproteins with one or more test substances under assay conditions suitable for the detection of inhibition or modulation of said secretion; and
(ii) measuring whether secretion of said mucins, O-glycosylated glycoproteins, glycoproteins and proteins are thereby inhibited or modulated by one or more of the substances.

Substances identified as agents which are effective in binding to one or more lectin domains of polypeptide GalNAc-transferases using the above method of the invention may e.g. be selected from the group consisting of naturally or non-naturally occurring carbohydrates, peptides, glycopeptides, glycoconjugates and portions and fragments thereof. They may also be found among nucleic acids as well as small organic or inorganic molecules. They include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)2, and Fab expression library fragments, and epitope-binding fragments thereof)], and polypeptides. A substance to be used as an agent according to the invention may be an endogenous physiological compound or it may be a natural or synthetic compound.

Agents in accordance with the present invention are useful for changing the density and sites of O-glycan occupancy in mucins and O-linked glycoproteins. Further uses are in changing Golgi-transport and intracellular sorting events conferred by the lectin domains of GalNAc-transferases. For example, inhibitors of lectin domains of GalNAc-transferases may be useful in manipulating disease associated O-glycosylation to augment immunity and to prepare vaccines. Further use may be found in manipulating mucin secretion and O-glycan density in diseases associated with mucous accumulation to decrease secretion and enhance clearance of mucins. Further use may entail modulating O-glycosylation of recombinant glycoproteins by inhibition of polypeptide GalNAc-transferases in host expression cells. These and other aspects of the present invention will become evident upon reference to the following detailed description and drawings.

### BRIEF DESCRIPTION OF THE DRAWINGS

FIG. 1 is a schematic of the predicted domain structure of polypeptide GalNAc-transferases³¹. Polypeptide GalNAc-transferases are predicted to be type II transmembrane proteins with a cytoplasmic N-terminal tail, a non-cleaved hydrophobic transmembrane retention signal (TM, grey box), a stem region of variable size, a well conserved catalytic unit of approximately 340 amino acid residues, and a poorly conserved C-terminal domain of approximately 130 amino acids which display structural similarity to the lectin ricin and have been designated the “lectin domain”. The putative lectin domains consist of α-, β-, and γ-ricin-like repeats. C indicates conserved cysteine residues among sequences.

FIG. 2 is a multiple sequence alignment (ClustalW) of putative lectin domains derived from 16 human polypeptide GalNAc-transferases. Positions of conserved motifs CLD and QxW in the α, β, and γ repeats are indicated. The numbering indicated in the margins reflects numbering of the analysed sequence region of each GalNAc-transferase. Conserved residues are indicated by black box'ing.

FIGS. 3A and B are schematic representations of human soluble secreted MUC1 expression constructs used for stable transfectants of CHO cells. Panel A: IgG2A His-tag was inserted into Bsu36I/XbaI site of MUC1FL, generating a His-tagged MUC1 construct containing the endogenous MUC1 secretion signal peptide. Panel B: Muc1FL Sau3AI insert was inserted into the BamHI site of pcDNA-inf., generating a non-tagged MUC1 construct containing the γ-interferon secretion signal peptide.

FIG. 4 is a plot of absorbance v. ligand dilution showing GalNAc-transferase binding to GalNAc-MUC1 glycopeptide. A direct binding assay (ELISA) mediated by the lectin domain was developed and validated with soluble secreted GalNAc-T4 and -T2 enzyme proteins. ELISA plates were coated with peptides or glycopeptides at 1 μg/ml, blocked with BSA, and incubated with biotinylated enzymes. After washing, bound enzyme proteins were detected with HRP-Streptavidin as described in detail in Example 2. Secreted soluble constructs of GalNAc-transferases which are enzymatically active may bind to (glyco)peptide substrates through their catalytic units as originally described for GalNAc-T2³². However, GalNAc-transferase binding to substrates by the catalytic domain requires UDP and divalent cat-ions (binding destroyed by EDTA treatment), in accordance with previous experience³². Panel A: GalNAc-T4 wild type enzyme proteins (□) and GalNAc-T2 (▪) selectively bind GalNAc-MUC1 glycopeptides, with no significant binding observed to the unglycosylated peptide (GalNAc-T4 wt (Δ) and GalNAcT2 (▴)). Panel B: Furthermore, the GalNAc-T4 lectin mutant did not bind to either glycosylated Muc1 (GalNAc-Muc1) (●) or non glycosylated Muc1 (Muc1) (∘), whereas GalNAc-T4 wild type binds GalNAc-Muc1 (□) but not nonglcosylated Muc1 (▪). Binding was not affected by 10 mM EDTA. Soluble secreted GalNAc-T4 mutant, GalNAc-T4^{459H 2}, in which the lectin domain has been selectively inactivated by a single amino acid substitution, showed no binding demonstrating that the binding observed with the wild type enzyme is mediated through the lectin domain.

FIG. 5 is a plot (absorbance at 495 v. concentration of inhibitor) showing inhibition of GalNAc-T4 lectin binding. Direct binding assays were performed with preincubation of GalNAc-T4 with inhibitors followed by incubation of GalNAc-T4 in ELISA plates activated with GalNAc-Muc1 as described in detail in Example 2. GalNAcα-benzyl (▪) as well as GalNAcβ-benzyl (□) inhibit at 3-6 mM, whereas the control GlcNAcα-benzyl (▴) showed no inhibition. This demonstrates that the GalNAc-transferase lectin domains show no specificity for the anomeric configuration of GalNAc, and identifies a novel inhibitor, GalNAcβ-benzyl, of GalNAc-transferase lectins.

FIGS. 6 A-J are a series of photomicrographs showing immunostaining of wild type CHO and transfected wild type CHO cells with a secreted MUC1 construct. CHO1d1D/MUCsol-cloneD5 was established from this population. MUC1 expression in the cytoplasm of 10-20% cells is visualized by HMFG2, SM3, and vu-4H5 antibodies. Anti-T antibody HH8 reacted only after neuraminidase pretreatment and the anti-Tn antibody reacted similarly before and after neuraminidase treatment. This suggests that cells grown in GalNAc alone produce mainly the Tn glycoform of MUC1, while cells grown in Gal and GalNAc produce mainly the sialylated T (core 1) glycoforms.

FIG. 7 is a series of SDS-PAGE Western analysis of MUC1 secreted from wild type CHO cells stably transfected cells with a secreted MUC1 construct (CHO1d1D/MUCsol-cloneD5). Neu+ indicates pretreatment of samples with neuraminidase as described in Examples. Cells were grown in culture medium after 24 or 48 hours analysed directly or after neuraminidase treatment.

FIG. 8 A-X (left to right from top to bottom) is a series of photomicrographs showing immunostaining of CHO 1d1D cells stably transfected cells with a full coding cell surface secreted MUC1 construct. CHO1d1D/MUC1F-clone2 cells were grown in Optimem medium without and with 1 mM GalNAc, and 1 mM GalNAc plus 0.1 mM Gal for 24-48 hours. Cells were trypsinized, washed, air-dried on cover slides, and immunostained as described in Examples with antibodies to MUC1 and T and Tn carbohydrates. Reactivity was evaluated before and after neuraminidase treatment of dried acetone fixed cells. +/−neu indicates that the staining was identical with or without neuraminidase pretreatment.

FIGS. 9A and B are SDS-PAGE Western analysis of MUC1 secreted from CHO 1d1D cells stably transfected cells with a secreted MUC1 construct. CHO1d1D/MUCsol-cloneD5 cells were grown in the presence or absence of sugars indicated, and samples of the culture medium analysed directly after 24-48 hours. Positive control (GalNAc-peptide) is a 60-mer MUC1 tandem repeat GalNAc-glycopeptide glycosylated with human polypeptide GalNAc-transferase GalNAc-T2. Lane labeled control includes medium from CHO 1d1D cells. Anti-MUC1 monoclonal antibodies 5E5 and HMFG2 were used.

FIGS. 10A and B are SDS-PAGE Western analysis of MUC1 secreted from CHO 1d1D cells stably transfected cells with a secreted MUC1 construct. Same experiment as FIG. 9 using anti-MUC1 monoclonal antibodies VU-4H5 and VU-2G7.

FIGS. 11A-D are a series of photomicrographs (left to right from top to bottom of) anti-MUC1 antibody immunofluorescence staining of CHO 1d1D cells stably transfected with a full coding cell surface MUC1 (CHO1d1D/MUC1F-clone2). Cell grown in the presence of GalNAc were treated with the O-glycosylation inhibitor GalNAcα-benzyl or control GlcNAcα-benzyl. Cells were grown in plates and stained without permeabilization as described in Example 4.

FIG. 12 is an SDS-PAGE Western analysis of GalNAcα-benzyl inhibition of MUC1 expression in CHO 1d1D cells stably transfected with a full coding MUC1 construct. Cells were grown for 24 hours (lanes 1-6) or 48 hours (lanes 7-12) in the presence of 1 mM GalNAc (lanes 1-3 and 7-9) or 1 mM GalNAc and 0.1 mM Gal (lanes 4-6 and 10-12) to limit core O-glycosylation to GalNAcα1-O-Ser/Thr and Galβ1-3GalNAcα1-O-Ser/Thr, respectively. Cells were further treated with 2 mM GalNAcα-benzyl (lanes 1, 4, 7, 10), 2 mM GlcNAcα-benzyl (lanes 2, 5, 8, 11) or no inhibitor (lanes 3, 6, 9, 12). Cells were washed and lysed at 24 or 48 hours and the lysates subjected to immunoprecipitation with monoclonal antibody HMFG2, which broadly recognize MUC1 glycoforms. Immunoprecipitates were analysed by SDS-PAGE and western blot using HMFG2 antibody to detect MUC1 expression. Lane M indicates molecular markers with assigned mw. Lane C includes a control MUC1 180-mer tandem repeat peptide which has been GalNAc-glycosylated with 3 moles GalNAc per repeat using GalNAc-T2. The sharp bands migrating at 100-200 kd are immunoglobulins indicated by IgG. At 24 hours the MUC1 glycoforms expressed by cells grown in GalNAc or Gal and GalNAc migrated similarly, indicating that synthesis of sialylated core 1 O-glycans were time limited (lanes 1-6). At 48 hours, MUC1 glycoforms migrating as higher molecular weight species were expressed by cells grown in Gal and GalNAc (lanes 11-12). Treatment with GalNAcα-benzyl had no significant effect at 24 hours (lanes 1 and 4), but after 48 hours a significant reduction in MUC1 expression was found in cells grown in GalNAc as well as in Gal and GalNAc (lanes 7 and 10). In the latter case a significant shift in migration further confirmed that GalNAcα-benzyl also serves as an inhibitor of O-glycan extension and reduces O-glycosylation to GalNAcα1-O-Ser/Thr. GlcNAcα-benzyl served as a control and had no effect on MUC1 expression and O-glycosylation compared to untreated cells (lanes 8 and 11).

FIG. 13 is a SDS-PAGE Western analysis resulting from the same experiment as in FIG. 12, but using a novel monoclonal antibody, 5E5, to visualize MUC1 expression. Cells were grown for 48 hours in the presence of 1 mM GalNAc (lanes 1-3) or 1 mM GalNAc and 0.1 mM Gal (lanes 4-6) to limit core O-glycosylation to GalNAcα1-O-Ser/Thr and Galβ1-3GalNAcα1-O-Ser/Thr, respectively. Cells were further treated with 2 mM GalNAcα-benzyl (lanes 1, 4), 2 mM GlcNAcα-benzyl (lanes 2, 5) or no inhibitor (lanes 3, 6). Cells were washed and lysed at 48 hours and the lysates subjected to immunoprecipitation with monoclonal antibody HMFG2, which broadly recognize MUC1 glycoforms. Immunoprecipitates were analysed by SDS-PAGE and western blot using 5E5 antibody, which selectively recognize GalNAc-glycosylated MUC1 expression and show no reactivity with unglycosylated MUC1 peptides. Lanes M and C as described in legend to FIG. 12. Treatment with GalNAcα-benzyl produced a significant reduction in MUC1 expression in cells grown in GalNAc as well as in Gal and GalNAc (lanes 1 and 4). In cells grown in Gal and GalNAc (lanes 4-6) only weak expression of MUC1 was detected, but treatment of cells with GalNAcα-benzyl still produced a marked shift in migration to lower molecular weight migrating species

FIG. 14 is an SDS-PAGE Western analysis showing the identification of a novel inhibitor, GalNAcβ-benzyl, which exhibits the same effect on mucin transport as GalNAcα-benzyl, but does not affect O-glycan extension and O-glycosylation in general. CHO 1d1D cells stably transfected with a full coding MUC1 construct were grown for 36 hours in the presence of 1 mM GalNAc (lanes 1-3 and 7-9) or 1 mM GalNAc and 0.1 mM Gal (lanes 4-6 and 10-12) to limit core O-glycosylation to GalNAcα1-O-Ser/Thr and Galβ1-3 GalNAcα1-O-Ser/Thr, respectively. Cells were further treated with 2 mM GalNAcα-benzyl (lanes 1, 4, 7, 10), 2 mM GalNAcβ-benzyl (lanes 2, 5, 8, 11) or 2 mM GlcNAcα-benzyl (lanes 3, 6, 9, 12). Cells were washed and lysed at 36 hours and the lysates subjected to immunoprecipitation with monoclonal antibodies HMFG2 (lanes 1-6) or 5E5 (lanes 7-12). Immunoprecipitates were analysed by SDS-PAGE and western blot using HMFG2 antibody. Lanes M and C as described in legend to FIG. 7. Treatment with GalNAcβ-benzyl produced the same or better reduction in MUC1 expression as treatment with GalNAcα-benzyl in cells grown in GalNAc as well as in Gal and GalNAc (lanes 2, 5, 8). In cells grown in Gal and GalNAc (lanes 4-6) MUC1 expression was reduced with GalNAcβ-benzyl treatment (lane 5), but in contrast to cells treated with GalNAcα-benzyl (lane 4), GalNAcβ-benzyl produced no change in the migration of MUC1 demonstrating that this inhibitor does not affect O-glycosylation. The lack of immunoprecipitation of MUC1 by 5E5 in cells grown in Gal and GalNAc (lanes 10-12) indicates that MUC1 is glycosylated with more complex structures than GalNAcα1-O-Ser/Thr as recognized by this antibody.

FIG. 15 is an SDS-PAGE Western analysis resulting from the same experiment as FIG. 14, except that expression is visualized by the monoclonal antibody 5E5. Cells were grown for 36 hours in the presence of 1 mM GalNAc (lanes 1-3 and 7-9) or 1 mM GalNAc and 0.1 mM Gal (lanes 4-6 and 10-12) to limit core O-glycosylation to GalNAcα1-O-Ser/Thr and Galβ1-3GalNAcα1-O-Ser/Thr, respectively. Cells were further treated with 2 mM GalNAcα-benzyl (lanes 1, 4, 7, 10), 2 mM GalNAcβ-benzyl (lanes 2, 5, 8, 11) or 2 mM GlcNAcα-benzyl (lanes 3, 6, 9, 12). Cells were washed and lysed at 36 hours and the lysates subjected to immunoprecipitation with monoclonal antibodies HMFG2 (lanes 1-6) or 5E5 (lanes 7-12). Immunoprecipitates were analysed by SDS-PAGE and western blot using 5E5 antibody. Lanes M and C as described in legend to FIG. 12. Treatment with GalNAcβ-benzyl produced the same or better reduction in MUC1 expression as treatment with GalNAcα-benzyl in cells grown in GalNAc (lanes 2 and 8). The lack of immunostaining of MUC1 by 5E5 in cells grown in Gal and GalNAc indicates that MUC1 is glycosylated with more complex structures than GalNAcα1-O-Ser/Thr as recognized by this antibody.

FIGS. 16A-O (left to right from top to bottom) is a series of photomicrographs showing that the main O-glycan phenotype of CHO 1d1D cells grown in Gal and GalNAc is sialylated T and that the 5E5 antibody does not react with MUC1, with T or sialylated T glycoforms of MUC1.

FIG. 17 illustrates SDS-PAGE Western analysis of MUC5AC secretion from HT29MTX cells using polyclonal anti-MUC5AC antibody LUM5-1. In order to evaluate GalNAcβ-benzyl as an inhibitor of mucin glycosylation and secretion we used the HT-29 metotrexate (MTX) cultured colon carcinoma cells of mucin secreting phenotype. HT-29 MTX cells have a goblet cell like phenotype with constitutive production of MUC5AC. Cells were continuously grown up to 21 days in the presence of 5 mM GalNAcα-benzyl, 5 mM GalNAcβ-benzyl, 5 mM GlcNAcα-benzyl and without inhibitor. SDS-PAGE Western blotting of media (10 μL) using the polyclonal anti-MUC5AC antibody LUM5-1 detected MUC5AC in media from untreated cells (Lane 1) and cells treated with GlcNAcα-benzyl (Lane 2). In striking contrast, MUC5AC was not detected in the media from cells treated with GalNAcα-benzyl (Lane 3) or GalNAcβ-benzyl (Lane 4). Results shown are from media collected at 14 days.

FIG. 18 illustrates immuno-fluorescence staining of HT29MTX cells treated with inhibitors as indicated and stained with the anti-MUC5AC monoclonal antibody, CLH2. Treatment of cells with GalNAcα-benzyl (Panel A) induced a storage phenotype with increased intracellular staining of MUC5AC. Cells treated with GalNAcβ-benzyl (Panel B) did not lead to a storage disease phenotype, and furthermore a clearly diminished intracellular staining of MUC5AC was found compared to cells treated with GlcNAcα-benzyl (Panel C) and untreated cells (Panel D).

### DETAILED DESCRIPTION OF THE INVENTION

All patent applications, patents, and literature references cited in this specification are hereby incorporated by reference in their entirety. In the case of conflict, the present description, including definitions, is intended to control.

### 1. DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the term “about” or “approximately” means within 50% of a given value, preferably within 20%, more preferably within 10%, more preferably still within 5%, and most preferably within 1% of a given value. Alternatively, the term “about” or “approximately” means that a value can fall within a scientifically acceptable error range for that type of value, which will depend on how qualitative a measurement can be given the available tools. “About” or “approximately” may define a distribution around a mean value, rather than a single value.

Molecular Biology Definitions. In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

“Nucleic acid” or “polynucleotide” as used herein refers to purine- and pyrimidine-containing polymers of any length, either polyribonucleotides or polydeoxyribonucleotides or mixed polyribo-polydeoxyribo nucleotides. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases (see below).

“Complementary DNA or cDNA” as used herein refers to a DNA molecule or sequence that has been enzymatically synthesised from the sequences present in an mRNA template, or a clone of such a DNA molecule. A “DNA Construct” is a DNA molecule or a clone of such a molecule, either single- or double-stranded, which has been modified to contain segments of DNA that are combined and juxtaposed in a manner that would not otherwise exist in nature. By way of non-limiting example, a cDNA or DNA which has no introns are inserted adjacent to, or within, exogenous DNA sequences.

A plasmid or, more generally, a vector, is a DNA construct containing genetic information that may provide for its replication when inserted into a host cell. A plasmid generally contains at least one gene sequence to be expressed in the host cell, as well as sequences that facilitate such gene expression, including promoters and transcription initiation sites. It may be a linear or closed circular molecule.

Nucleic acids are “hybridizable” to each other when at least one strand of one nucleic acid can anneal to another nucleic acid under defined stringency conditions. Stringency of hybridization is determined, e.g., by a) the temperature at which hybridization and/or washing is performed, and b) the ionic strength and polarity (e.g., formamide) of the hybridization and washing solutions, as well as other parameters. Hybridization requires that the two nucleic acids contain substantially complementary sequences; depending on the stringency of hybridization, however, mismatches may be tolerated. Typically, hybridization of two sequences at high stringency (such as, for example, in an aqueous solution of 0.5×SSC, at 65° C.) requires that the sequences exhibit some high degree of complementarity over their entire sequence. Conditions of intermediate stringency (such as, for example, an aqueous solution of 2×SSC at 65° C.) and low stringency (such as, for example, an aqueous solution of 2×SSC at 55° C.), require correspondingly less overall complementarily between the hybridising sequences. Hybridization stringency has been defined in numerous publication known to the skilled in the art (Meinkoth and Wahl, Anal. Biochem. 138, 267-284, 1984; Maniatis et al., Molecular cloning: a laboratory manual. Cold Spring Harbor Laboratory Press, 1989; J. Q. Zhang, Eur. J. Biochem. 239, 835-841:1996; M. Friedman-Einat, General and Comparative Endocrinology 115, 354-363:1999: M. Szabo, J. Bacteriology, 1544-1553:1995; S. Sau, J. Bacteriology, 21182126, 1996). Nucleic acids are “hybridizable” to each other when at least one strand can anneal to another nucleic acid under defined stringency conditions. High stringency hybridization is defined as 42° C. over night hybridization under standard conditions (Maniatis et al., Molecular cloning: a laboratory manual. Cold Spring Harbor Laboratory Press, 1989), followed by 5 washes with 2×SSC, 0.1% SDS at 42° C., once with 0.5×SSC, 0.1% SDS at 55° C. and once with 0.1×SSC, 0.1% SDS at 55° C. (1×SSC is 0.15M NaCl, 0.015M Na citrate). Northern and Southern nucleic acid blotting hybridization techniques, especially for the purpose of investigating hybridization specificity, is well known to those skilled in the field of the invention.

An “isolated” nucleic acid or polypeptide as used herein refers to a component that is removed from its original environment (for example, its natural environment if it is naturally occurring). An isolated nucleic acid or polypeptide contains less than about 50%, preferably less than about 75%, and most preferably less than about 90%, of the cellular components with which it was originally associated.

A “probe” refers to a nucleic acid that forms a hybrid structure with a sequence in a target region due to complementarily of at least one sequence in the probe with a sequence in the target region.

A nucleic acid that is “derived from” a designated sequence refers to a nucleic acid sequence that corresponds to a region of the designated sequence. This encompasses sequences that are homologous or complementary to the sequence, as well as “sequence-conservative variants” and “function-conservative variants”. Sequence-conservative variants are those in which a change of one or more nucleotides in a given codon position results in no alteration in the amino acid encoded at that position.

Function-conservative variants of polypeptide GalNAc-transferases are those in which a given amino acid residue in the polypeptide has been changed without altering the overall conformation and enzymatic activity (including substrate specificity) of the native polypeptide; these changes include, but are not limited to, replacement of an amino acid with one having similar physico-chemical properties. This includes but is not limited to, replacement of an amino acid with one having similar structural or physical properties, including polar or non-polar character, size, shape and charge (see, e.g., Table A).

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “mutation” means any process or mechanism resulting in a mutant protein, enzyme, polypeptide, polynucleotide, gene, or cell. This includes any mutation in which a protein, enzyme, polynucleotide, or gene sequence is altered, and any detectable change in a cell arising from such a mutation. The altered protein, enzyme, polypeptide or polynucleotide is a “mutant”, also called a “variant.” Typically, a mutation occurs in a polynucleotide or gene sequence, by point mutations (substitutions), deletions, or insertions of single or multiple nucleotide residues. A mutation includes polynucleotide alterations arising within a protein-encoding region of a gene as well as alterations in regions outside of a protein-encoding sequence, such as, but not limited to, regulatory or promoter sequences. A mutation in a gene can be “silent”, i.e., not reflected in an amino acid alteration upon expression, leading to a “sequence-conservative” variant of the gene. This generally arises when one amino acid corresponds to more than one codon. Table A outlines which amino acids correspond to which codon(s).

Thus, due to the degeneracy of the genetic code, any three-nucleotide codon that encodes the GalNAc-transferase lectin domain polypeptides described herein is within the scope of the invention.

The terms “mutant” and “variant” may also be used to indicate a modified or altered gene, DNA or RNA sequence, enzyme, cell, etc., i.e., any kind of mutant. Such changes also include changes in the promoter, ribosome binding site, etc.

As outlined above, amino acid substitutions are generally based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

In addition, modifications, which do not normally alter the primary sequence of the GalNAc-transferase lectin domain polypeptides, include in vivo or in vitro chemical derivatization of polypeptides, e.g., acetylation, methylation, or carboxylation. Also included as variant polypeptides of this invention are these polypeptides modified by glycosylation, e.g., those made by modifying the glycosylation patterns of a polypeptide during its synthesis and processing or in further processing steps; or by exposing the polypeptide to enzymes which affect glycosylation, such as mammalian glycosylating or deglycosylating enzymes. Also embraced as variant polypeptides are the above-identified mutagenized sequences, which have phosphorylated amino acid residues, e.g., phosphotyrosine, phosphoserine, or phosphothreonine.

TABLE A

Amino Acids, Corresponding Codons, and
Functionality/Property

| | | | Side |
|---|---|---|---|
| Amino | | | Chain |
| Acid | SLC | DNA codons | Property |
| Isoleu- | I | ATT, ATC, ATA | Hydro- |
| cine | | | phobic |
| Leucine | L | CTT, CTC, CTA, CTG, TTA, TTG | Hydro- |
| | | | phobic |
| Valine | V | GTT, GTC, GTA, GTG | Hydro- |
| | | | phobic |
| Phenyla- | F | TTT, TTC | Aromatic |
| nine | | | side |
| | | | chain |
| Methio- | M | ATG | Sulphur |
| nine | | | group |
| Cy- | C | TGT, TGC | Sulphur |
| steine | | | group |
| Alanine | A | GCT, GCC, GCA, GCG | Hydro- |
| | | | phobic |
| Glycine | G | GGT, GGC, GGA, GGG | Hydro- |
| | | | phobic |
| Proline | P | CCT, CCC, CCA, CCG | Secon- |
| | | | dary |
| | | | amine |
| Threo- | T | ACT, ACC, ACA, ACG | Alipha- |
| nine | | | tic |
| | | | hydroxyl |
| Serine | S | TCT, TCC, TCA, TCG, AGT, AGC | Alipha- |
| | | | tic |
| | | | hydroxyl |
| Tyrosine | T | TAT, TAC | Aromatic |
| | | | side |
| | | | chain |
| Trypto- | W | TGG | Aromatic |
| phan | | | side |
| | | | chain |
| Gluta- | Q | CAA, CAG | Amide |
| mine | | | group |
| Aspara- | N | AAT, AAC | Amide |
| gine | | | group |
| Histi- | H | CAT, CAC | Basic |
| dine | | | side |
| | | | chain |
| Glutamic | E | GAA, GAG | Acidic |
| acid | | | side |
| | | | chain |
| Aspartic | D | GAT, GAC | Acidic |
| Acid | | | side |
| | | | chain |
| Lysine | K | AAA, AAG | Basic |
| | | | side |
| | | | chain |
| Arginine | R | CGT, CGC, CGA, CGG, AGA, AGG | Basic |
| | | | side |
| | | | chain |
| Stop | Stop | TAA, TAG, TGA | — |
| codons | | | |

As referred to herein, “sequence similarity” means the extent to which nucleotide or protein sequences are related. The extent of similarity between two sequences can be based on percent sequence identity and/or conservation. Amino acids other than those indicated as conserved may differ in a protein or enzyme so that the percent protein or amino acid sequence similarity between any two proteins of similar function may vary and can be, for example, at least 70%, preferably 75%, more preferably 80%, even more preferably 85%, and most preferably at least 90%, as determined according to an alignment scheme.

“Sequence identity” herein means the extent to which two nucleotide or amino acid sequences are invariant.

“Sequence alignment” means the process of lining up two or more sequences to achieve maximal levels of sequence identity (and, in the case of amino acid sequences, conservation), e.g., for the purpose of assessing the degree of sequence similarity. Numerous methods for aligning sequences and assessing similarity and/or identity are known in the art such as, for example, the ClustalW method, the Cluster Method, wherein similarity is based on the MEGALIGN algorithm, as well as BLASTN, BLASTP, and FASTA (Lipman and Pearson, 1985; Pearson and Lipman, 1988). When using all of these programs, the preferred settings are those that result in the highest sequence similarity.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

A “donor substrate” is a molecule recognised by, e.g., a polypeptide GalNAc-transferees and that contributes a N-acetylgalactosamine moiety for the transferase reaction. For polypeptide GalNAc-transferases, a donor substrate is UDP-N-acetylgalactosamine or with some GalNAc-transferase isoforms UDP-galactose. An “acceptor substrate” is a molecule, preferably a peptide, protein, glycopeptide, and glycoprotein, that is recognised by, e.g., a polypeptide GalNAc-transferase and that is the target for the modification catalysed by the transferase, i.e., receives the carbohydrate moiety. For polypeptide GalNAc-transferases, acceptor substrates include without limitation peptides, proteins, glycopeptides, and glycoproteins.

The term “agonist” refers to a molecule that increases the amount of, or prolongs the duration of, the activity of the polypeptide. The term “enhancer” refers to a molecule that similarly increases the amount of, or prolongs the duration of, the activity of the polypeptide. The term “antagonist” refers to a molecule, which decreases the biological or immunological activity of the polypeptide. The term “inhibitor” similarly refers to a molecule, which decreases the biological or immunological activity of the polypeptide. Agonists, antagonists, and inhibitors may include proteins, nucleic acids, carbohydrates, or any other molecules that associate with a polypeptide GalNAc-transferase.

The term “agent” includes small molecules, peptide mimetics and polypeptides.

“Mimetics” of GalNAc-transferase lectin-domain inhibitors are molecules that functionally mimic the structure or function of a GalNAc-transferase lectin-domain inhibitor. Molecular mimetics include, but are not limited to: small organic compounds; nucleic acids and nucleic acid derivatives; saccharides or oligosaccharides; peptide mimetics including peptides, proteins, and derivatives thereof, such as peptides containing non-peptide organic moieties, synthetic peptides which may or may not contain amino acids and/or peptide bonds, but retain the structural and functional features of a peptide ligand; pyrrolidines; peptoids and oligopeptoids which are molecules comprising N-substituted glycine, such as those described by Simon et al. (1992) Proc. Natl. Acad. Sci. USA 89:9367.

The human N-acetylgalactosaminyltransferase T1 gene (GALNT1) has been described previously³². The sequence of the GALNT1 mRNA and the sequence of the GalNAc-T1 polypeptide have been submitted to GenBank/EBI Data Bank and assigned accession numbers X85018 and CAA59380, respectively.

The human N-acetylgalactosaminyltransferase T2 gene (GALNT2) has been described previously³². The sequence of the GALNT2 mRNA and the sequence of the GalNAc-T2 polypeptide have been submitted to GenBank/EBI Data Bank and assigned accession numbers X85019 and CAA59381, respectively.

The human N-acetylgalactosaminyltransferase T3 gene (GALNT3) has been described previously³³. The sequence of the GALNT3 mRNA and the sequence of the GalNAc-T3 polypeptide have been submitted to GenBank/EBI Data Bank and assigned accession numbers X92689 and CAA63371, respectively.

The human N-acetylgalactosaminyltransferase T4 gene (GALNT4) has been described previously¹⁰. The sequence of the GALNT4 mRNA and the sequence of the GalNAc-T4 polypeptide have been submitted to GenBank/EBI Data Bank and assigned accession numbers Y08564 and CAA69875, respectively.

The human N-acetylgalactosaminyltransferase T5 gene (GALNT5) has been described previously⁸. The sequence of the GALNT5 mRNA and the sequence of the GalNAc-T5 polypeptide have been submitted to GenBank/EBI Data Bank and assigned accession numbers AJ245539 and CAB65104, respectively.

The human N-acetylgalactosaminyltransferase T6 gene (GALNT6) has been described previously³⁴. The sequence of the GALNT6 mRNA and the sequence of the GalNAc-T6 polypeptide have been submitted to GenBank/EBI Data Bank and assigned accession numbers AJ133523 and CAB55325, respectively. The human N-acetylgalactosaminyltransferase T7 gene (GALNT7) has been described previously⁸. The sequence of the GALNT7 mRNA and the sequence of the GalNAc-T7 polypeptide have been submitted to GenBank/EBI Data Bank and assigned accession numbers AJ002744 and CAB60270, respectively.

The human N-acetylgalactosaminyltransferase T8 gene (GALNT8) has been described previously³⁵. The sequence of the GALNT8 mRNA and the sequence of the GalNAc-T8 polypeptide have been submitted to GenBank/EBI Data Bank and assigned accession numbers AJ271385 and CAB89199, respectively.

The human N-acetylgalactosaminyltransferase T9 gene (GALNT9) has been described previously³⁶. The sequence of the GALNT9 mRNA and the sequence of the GalNAc-T8 polypeptide have been submitted to GenBank/EBI Data Bank and assigned accession numbers AB040672 and BAB13699, respectively.

The human N-acetylgalactosaminyltransferase T10 nucleic acid sequence (GALNT10) and the sequence of the encoded GalNAc-T10 polypeptide have been submitted to GenBank/EBI Data Bank. The nucleic acid accession number is AJ505950.

The human N-acetylgalactosaminyltransferase T11 gene (GALNT11) has been described previously³. The sequence of the GALNT11 mRNA and the sequence of the GalNAc-T11 polypeptide have been submitted to GenBank/EBI Data Bank and assigned accession numbers Y12434 and CAC79625, respectively.

The human N-acetylgalactosaminyltransferase T12 nucleic acid sequence (GALNT12) and the sequence of the GalNAc-T12 polypeptide have been submitted to GenBank/EBI Data Bank. The nucleic acid accession number is AJ132365. This sequence is disclosed herewith.

The human N-acetylgalactosaminyltransferase T13 nucleic acid sequence (GALNT13) and the sequence of the GalNAc-T13 polypeptide have been submitted to GenBank/EBI Data Bank. The nucleic acid accession number is AR153422.

The references cited above for describing human GalNAc-T1-13 are incorporated herein by reference in their entirety and to the same extent as if each reference was individually incorporated by reference.

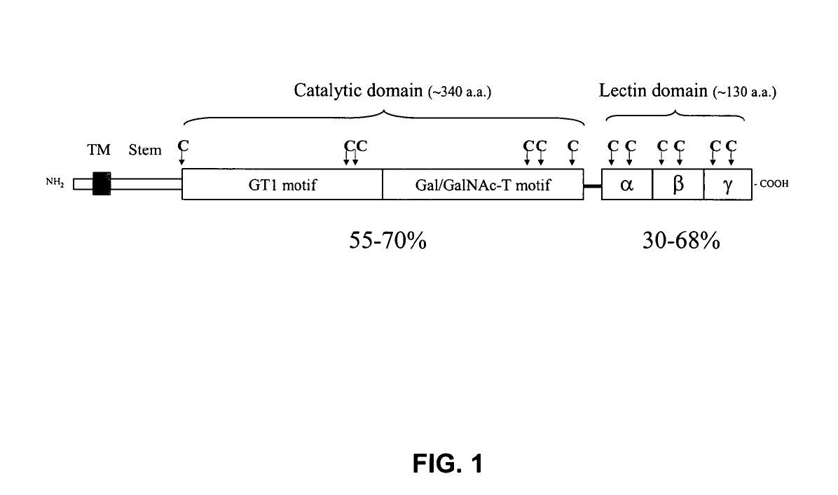

Expression to produce enzymatically-active polypeptide GalNAc-transferases can be carried out in any number of conventional expression systems familiar to those skilled in the art. In one embodiment, GalNAc-transferases are expressed in a secreted soluble form, which can be recovered from the culture medium. Such secreted enzymes lack the N-terminal cytoplasmic tail and transmembrane retention sequence, and have N-terminal sequence starting in the predicted stem region (depicted domain structures of polypeptide GalNAc-transferases shown in FIG. 1). The boundaries of the stem is N-terminally defined by the hydrophobic signal sequence, while the C-terminal boundary is less clearly defined but limited to the conserved catalytic unit of the enzymes as defined by multiple sequence alignments. For some isoforms including GalNAc-T2 the N-terminal sequence have been determined in naturally occurring soluble proteins derived from proteolytic cleavage³². In another embodiment, host cells (e.g. CHO cells) are engineered to express full coding GalNAc-transferases and glycosylate substrates in vivo in host cells.

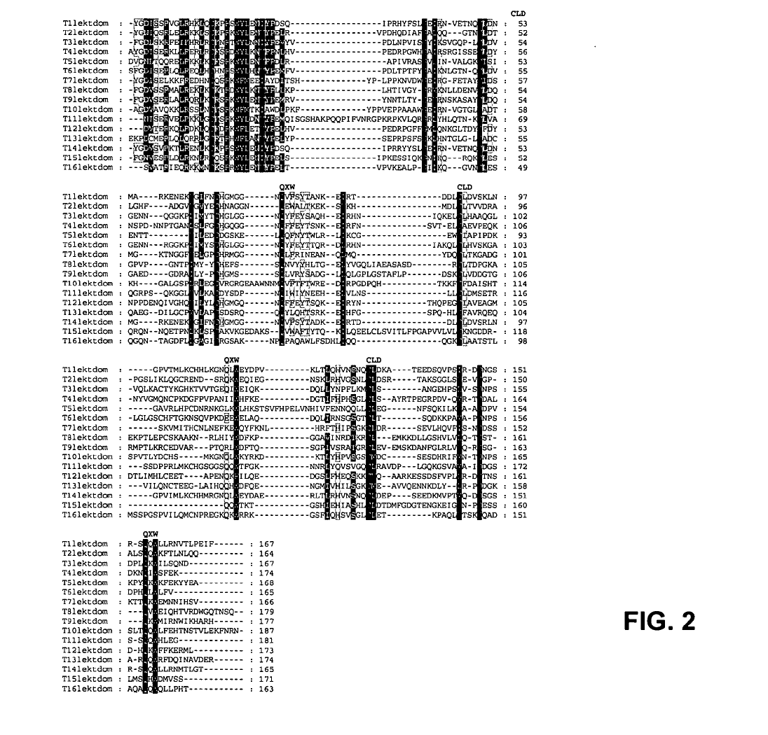

Expression to produce functional lectin domains of polypeptide GalNAc-transferases without the catalytic unit (or activity) can be carried out in any number of conventional expression systems familiar to those skilled in the art. In one embodiment, GalNAc-transferase lectins are expressed in a secreted soluble form, which can be recovered from the culture medium. Such secreted soluble forms lack the N-terminal cytoplasmic tail, transmembrane retention sequence, stem region and the catalytic unit. The boundaries of the catalytic units and lectin domains are defined by multiple sequence alignments and experimentation of lectin binding activity (multiple sequence alignment analysis of the C-terminal sequences polypeptide GalNAc-transferases including the most C-terminal boundaries of the catalytic domains and the entire lectin domains shown in FIG. 2). The boundaries cannot be clearly defined but the most C-terminal well-conserved sequence motif of the catalytic units (WYLENVYP) can be excluded from the lectin domains. Parts of or the entire catalytic domains may be included to produce functional lectin domains, and inclusion of inactivating mutations in the catalytic units (e.g. mutations in the D×H motif important for donor substrate binding, or residues important for acceptor substrate binding⁴) may be used to avoid additional binding activity mediated through the catalytic units. In another embodiment, host cells (e.g. CHO cells) are engineered to express full coding polypeptide GalNAc-transferases with or without mutations in their catalytic units and binding mediated through lectin domains are determined in vivo in host cells.

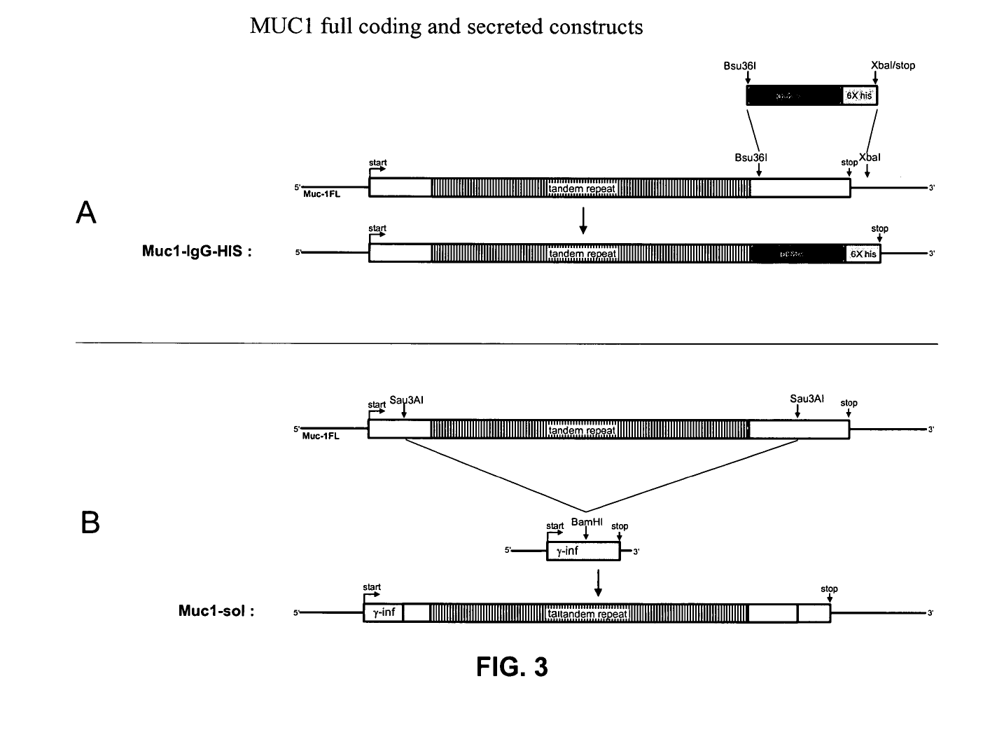

Cells stably or transiently transfected with full coding or secreted expression constructs of mucins, mucin-like glycoproteins, O-glycosylated proteins, or proteins can be carried out by any number of conventional methods familiar to those skilled in the art. In one embodiment, the mucin MUC1 is expressed in a soluble form, which can be recovered from the culture medium (FIG. 3 illustrates MUC1 expression constructs used in this invention; the DNA sequence is available from GenBank accession number M61170). In another embodiment, host cells (e.g. CHO or CHO 1d1D cells) are engineered to express MUC1 on the cell surface. In a preferred embodiment of the invention the cells are mammalian and more preferably, the cells are human.

Human cell lines expressing cell surface mucins or secreting mucins can be selected, cultured and treated by any number of conventional methods familiar to those skilled in the art. In one embodiment, mucins are expressed in a secreted soluble form without transmembrane retention sequence, which can be recovered from the culture medium. In another embodiment, host cells (e.g. CHO 1d1D cells) are engineered to express full coding mucins on the cell surface.2.

### 2. GENERAL ASPECTS OF THE INVENTION

The putative lectin domains of some GalNAc-transferases notably GalNAc-T4 and -T7 have previously been shown to be important for the GalNAc-glycopeptide substrate specificities exhibited by these GalNAc-transferase isofomis (PCT WO 01/82215). The mechanism by which the putative lectin domains exert this effect on the enzyme activities is unknown. However, because GalNAc and GalNAcα-benzyl were found to selectively inhibit these activities it was hypothesized that the putative lectin domains functioned by recognizing the sugar or glycopeptide in a lectin-like interaction². Considerable efforts have been applied to demonstrate actual binding without success in the past (Bennett et al. unpublished, personal communications).

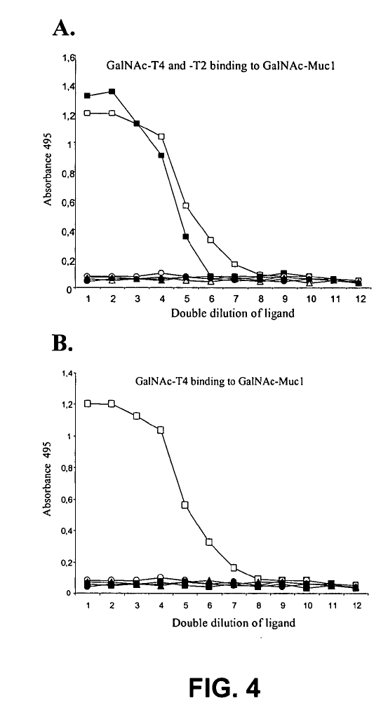

In the present invention a direct binding assay was developed using secreted soluble GalNAc-T4 and -T2, and chemoenzymatically produced multimeric MUC1 tandem repeat GalNAc-glycopeptides (FIG. 4). Short MUC1 glycopeptides of traditional length of 15-20 amino acids have failed to provide significant binding in the same assay system, and one improvement leading to the success was the application of extended multimeric MUC1 GalNAc-glycopeptides. Binding was also found to an enzymatically GalNAc-glycosylated fusion protein expressed in E. coli and containing 30 amino acids of the MUC2 tandem repeat. Another improvement was the use of biotinylation of the enzymes, which provided an improved signal compared to previous attempts with identifying retained or bound enzyme by measuring activity. The specific activities of GalNAc-transferases as measured in in vitro assays are relatively low, and in past attempts to use binding and elution of enzyme activity presumably the detection level was not sufficient to detect binding. The developed assay was validated to demonstrate binding through the lectin domains by several ways: i) binding was selective for GalNAc-glycosylated glycopeptides with no significant binding to unglycosylated peptides; ii) a single amino acid substitution in the lectin domains of GalNAc-T4 (and -T2), known to selectively destroy GalNAc-glycopeptide specificity of these enzymes without affecting the catalytic unit² abolished binding; iii) binding was not affected by EDTA treatment which is known to destroy catalytic activity of GalNAc-transferases^{32,37-39}; iv) binding was selectively inhibited by the monosacharide GalNAc and not by other sugars.

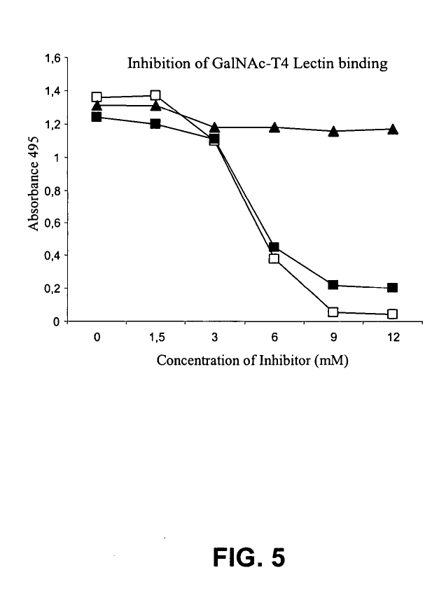

In order to minimize the size and functional complexity of GalNAc-transferase lectins to be used as probes for binding studies, we used multiple sequence alignment analysis to predict and design suitable expression constructs for isolated lectin domains (FIG. 2). In the present invention a direct binding assay was developed using isolated lectin domains of GalNAc-T4 and -T2 with minimal size (FIG. 5). Analysis of the fine specificity of the binding by inhibition studies showed that GalNAc-T2 and -T4 lectins exhibit restricted specificity for GalNAc structures, and surprisingly that the anomeric configuration of the GalNAc residue is not important. Thus, both GalNAcα- and GalNAcβ-benzyl inhibited binding to the same degree. The lectin Helix Pomatia (HP) was used as a control plant lectin with known binding specificity for GalNAcβ-structures. HP lectin showed a very different highly preferred binding specificity for GalNAcα-structures.

Studies with GalNAcα-benzyl have shown that this compound is effectively taken up by cells and used in the Golgi compartments²⁶. It is also well known in the art that sugar-aryl compounds are taken up by the cell and used in the Golgi compartments. Thus, the surprising finding that GalNAc-transferase lectins can be inhibited by βGalNAc (GalNAcβ-benzyl), provides a new tool to study the function of polypeptide GalNAc-transferases in vivo; GalNAcβ-benzyl, because it, too, will enter the cell and be used in the Golgi compartments.

Availability of a binding assay is a useful tool to identify and characterize inhibitors of GalNAc-transferase lectins. In accordance with the binding assay method of the invention, GalNAc-transferases are contacted with a glycopeptide, glycoprotein, fusionprotein, or other appropriate structure or polymer containing the sugar hapten structure recognized by the GalNAc-transferase lectins, preferably N-acetylgalactosamine, and the GalNAc-transferase protein or lectin bound is quantitatively measured. The GalNAc-transferases may be in the form of a secreted soluble construct as applied in this invention, and any extended or truncated construct of a GalNAc-transferase as well as hybrid fusion protein that maintains the binding properties of the lectin. The ligand may be in the form of a chemoenzymatically produced MUC1 GalNAc-glycopeptide as applied in this invention, and any glycopeptide, glycoprotein, fusionprotein of any size or sequence, or other appropriate structure or polymer containing the sugar hapten structure recognized by the GalNAc-transferase lectins. Synthesis and chemoenzymatic synthesis of glycopeptides are familiar to those in the art, and are described in the literature cited above and in the Examples provided below. The ligand sugar may be GalNAc, N-acetylgalactosamine, or any other sugar in any linkage and sequence recognized by a GalNAc-transferase lectin. The binding assay may be an enzyme-linked solid phase immunoadsorption assay (ELISA) as applied in the invention, and any variant assay hereof where binding to ligand can be detected including without limitation radioimmunoassay (RIA), surface plasmon resonance (SPR), chemoluminescense, nuclear magnetic resonance spectroscopy (¹H-NMR), and other methods know in the art. Binding may be detected by horse-radish-peroxidase HRP-Avidin biotin as applied in this invention, and any other detection system including without limitation enzyme reactions, fluorescence, radioactivity, spectroscopy, spectrometry and other methods. The GalNAc-transferases may be labelled by biotinylation as applied in this invention, and any other labelling including without limitation antibody tags, enzymes, fluorochromes, radioisotopes and other methods know in the art, as well as detected by antibodies, phage antibody fragments or other binding proteins. The assay may used to characterize binding specificities of GalNAc-transferase lectins, screen and identify inhibitors of GalNAc-transferase lectins, and screen and identify competitive binders such as different GalNAc-transferase lectins and other lectins and proteins with binding properties for carbohydrates.

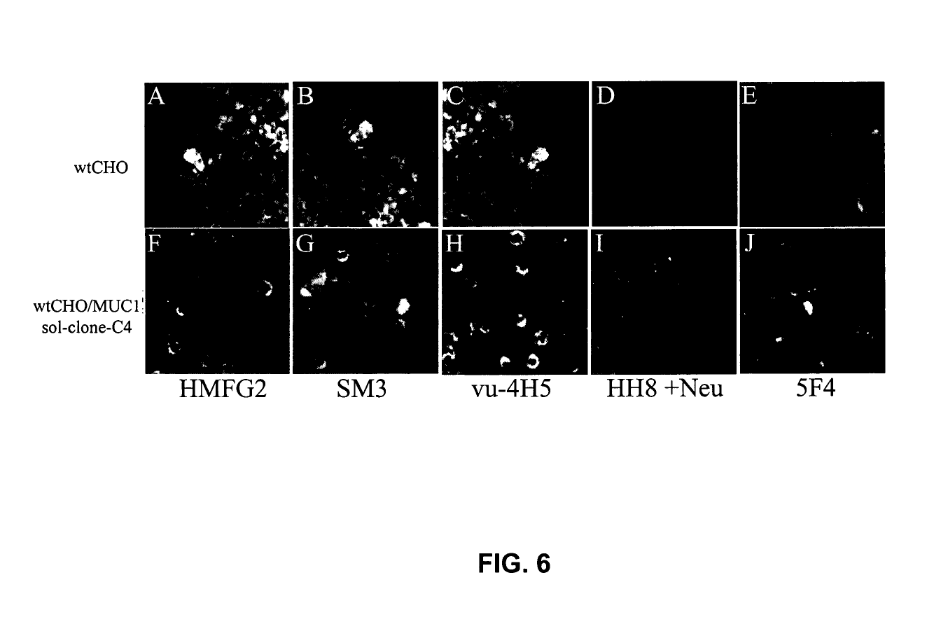

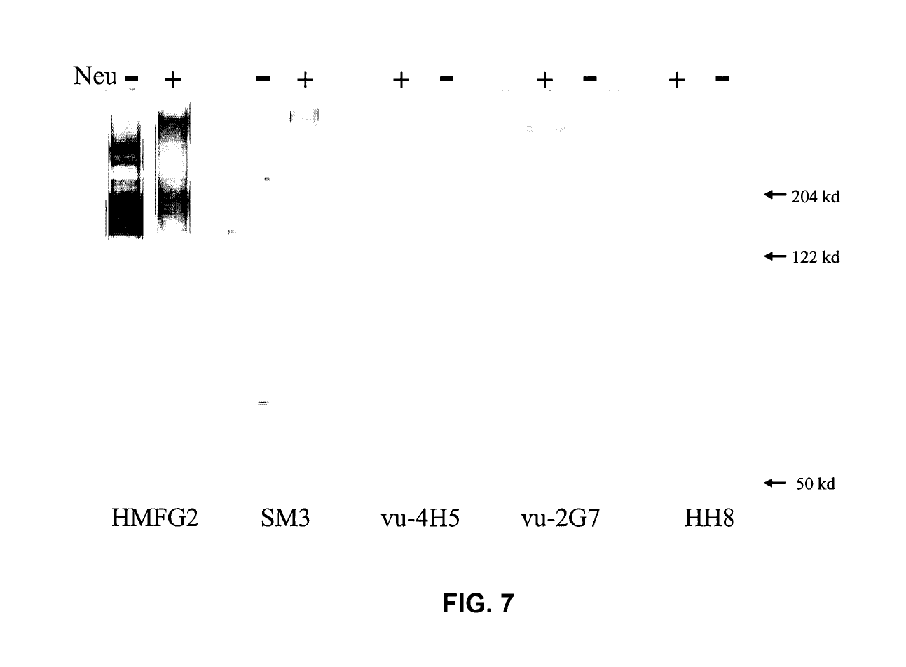

An in vivo model system for secretion of mucins was developed. A truncated secreted expression construct of the human cell surface mucin MUC1 containing 32 tandem repeats (FIG. 3), was stably transfected into CHO wild type and CHO 1d1D cells⁴⁰. FIG. 6 illustrates intracellular expression of soluble MUC1 in wild type CHO transfectant clone, wtCHO/MUC1sol-clone-C4, visualized by multiple monoclonal anti-MUC1 antibodies. Analysis of glycosylation was performed with a panel of antibodies with well-defined specificities for carbohydrate structures, and reactivity was mainly found with anti-T after pre-treatment with neuraminidase to remove sialic acids. Weak staining with anti-Tn was also found in some cells. FIG. 7 illustrates western blot analysis of secreted MUC1 from the same cells. High molecular weight MUC1 migrating with apparent mw higher than 300 Kd is labelled by HMFG2, SM3, and VU-2G7, while all antibodies including VU-4H5 label a low molecular weight MUC1 migrating with apparent mw of 130 Kd and presumed to represent virtually unglycosylated MUC1. Pretreatment with neuraminidase decreased migration of the high molecular weight MUC1 bands, and anti-T antibody reactivity emerged. Stable MUC1 transfectants in CHO 1d1D showed similar patterns of reactivity when grown in Gal and GalNAc.

An in vivo model system for cell surface expression of mucins was developed. A full coding expression construct of the human cell surface mucin MUC1 containing 32 tandem repeats (FIG. 3), was stably transfected into CHO wild type and CHO 1d1D cells. CHO 1d1D cells were originally established by Krieger et al.⁴⁰ and found to have a defect in UDP-Gal/GalNAc epimerase that renders the cells incapable of producing UDP-Gal and UDP-GalNAc. Lack of UDP-Gal limits the synthesis of all types of glycoconjugates including glycosphingolipids, N-linked and O-linked glycoproteins. The synthesis of O-linked glycoproteins will be arrested at GalNAcα1-O-Ser/Thr with or without addition of α2,6 linked sialic acid. In the absence UDP-GalNAc mainly O-linked mucin-type glycoconjugates are affected, and essentially no glycosylation occur, as the first sugar attached is GalNAc. The defect in CHO 1d1D cells can be selectively restored by addition of 1 mM GalNAc and or 0.1 mM Gal to the growth medium⁴⁰. Addition of both sugars essentially restores normal glycosylation, whereas addition of GalNAc alone limits O-glycosylation to GalNAcα1-O-Ser/Thr with or without addition of α2,6 linked sialic acid, and also affects galactosylation of N-linked glycosylation and glycolipid biosynthesis. Altschuler et al.⁴¹ have previously shown that cell surface expression of MUC1 in CHO 1d1D cells requires addition of GalNAc.

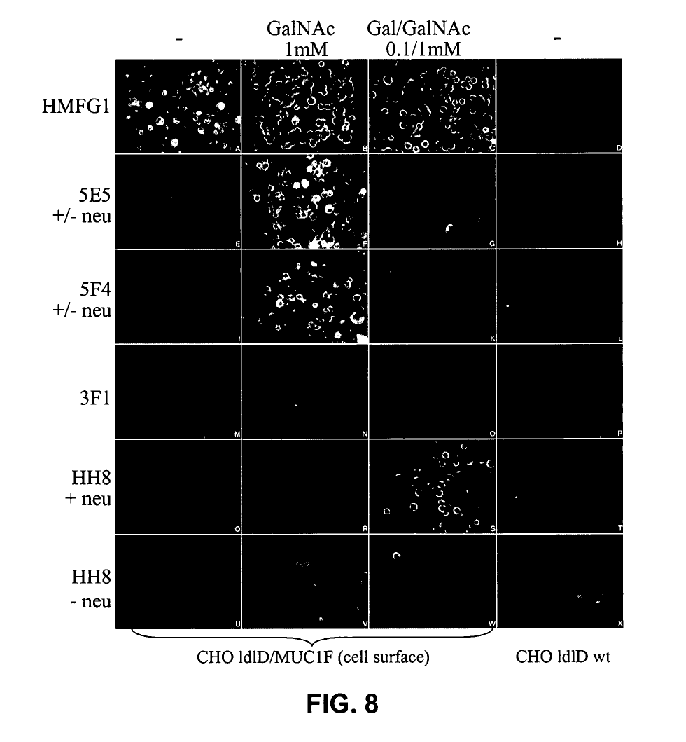

Cell surface expression of MUC1 was established in stably transfected CHO wildtype and CHO 1d1D cells. MUC1 was detected at the surface of non-permeabilized cells using monoclonal anti-MUC1 antibodies (FIG. 8). In accordance with Alschuler et al.⁴¹ MUC1 surface expression in CHO 1d1D cells was only found in cells grown in GalNAc or Gal and GalNAc, whereas cells grown without sugars or only in Gal failed to express MUC1 at the surface. In agreement with the conclusion drawn by Altchuler et al. 41 surface expression of MUC1 was dependent only on the first step in O-glycosylation, the addition of GalNAc.

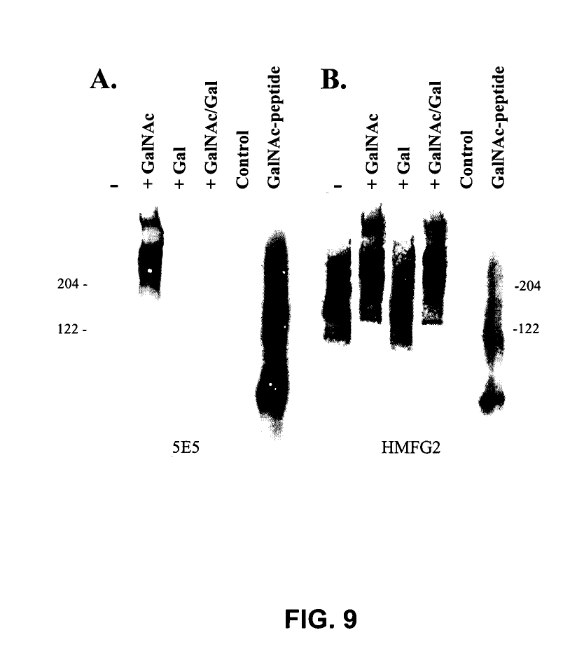

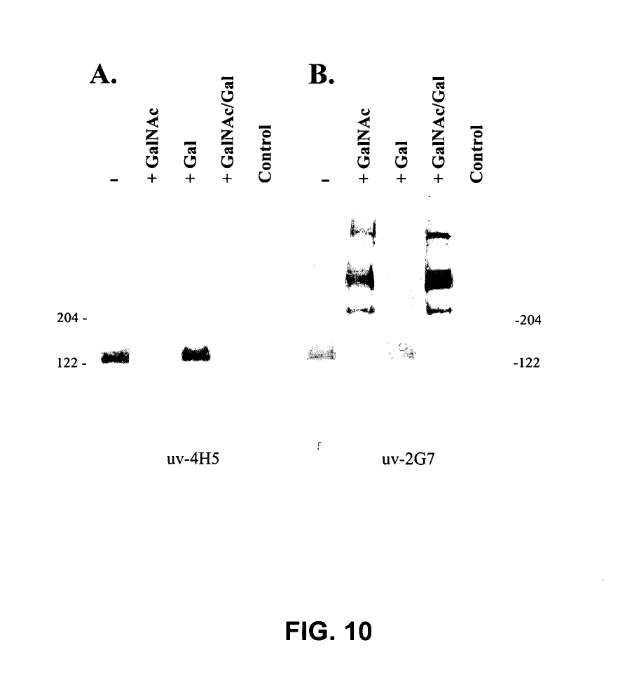

MUC1 produced in CHO 1d1D cells grown without GalNAc is not accumulated in Golgi, but degraded in lysosomes⁴¹. This indicates that measuring total MUC1 in cell lysates rather than exclusively at the cell surface may be used as a measure of MUC1 expression. The experiments shown in FIGS. 9-10 use immunoprecipitation of total cell lysates with anti-MUC1 antibody followed by western blot analysis with the same or different anti-MUC1 antibody to quantify and characterize MUC1 expression in cells. MUC1 produced in cell grown without GalNAc or only in the presence of Gal migrate close to the predicted mass of the protein core. With the addition of GalNAc to the medium high molecular weight forms of MUC1 are found, and these react with all antibodies except VU-2G7. The antibody 5E5 only reacts with Tn glycoforms and lack of reactivity with MUC1 from cells grown in Gal and GalNAc indicate that the majority of MUC1 produced is glycosylated with sialyl-T structures.

GalNAcα-benzyl is a well-known inhibitor of O-glycosylation extension²⁶. Treatment of cells with 1-2 mM GalNAcα-benzyl partially blocks core 1 O-glycosylation including α2,3 sialylation. Treatment of cells with GalNAcα-benzyl is also known to affect surface expression of mucins and O-glycosylated glycoproteins, as well as in some cases secretion of mucins. A number of mammalian cell lines have been treated with 1-2 mM GalNAcα-benzyl in the past and the resulting effects on O-glycosylation as well as mucin secretion have varied with cell type (for a detailed review see⁴²).

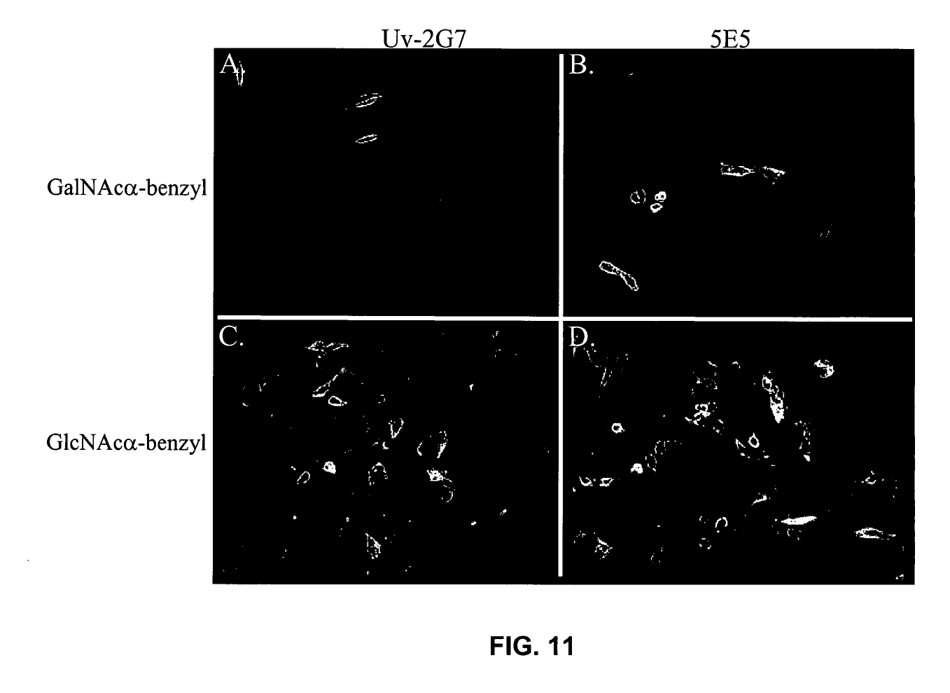

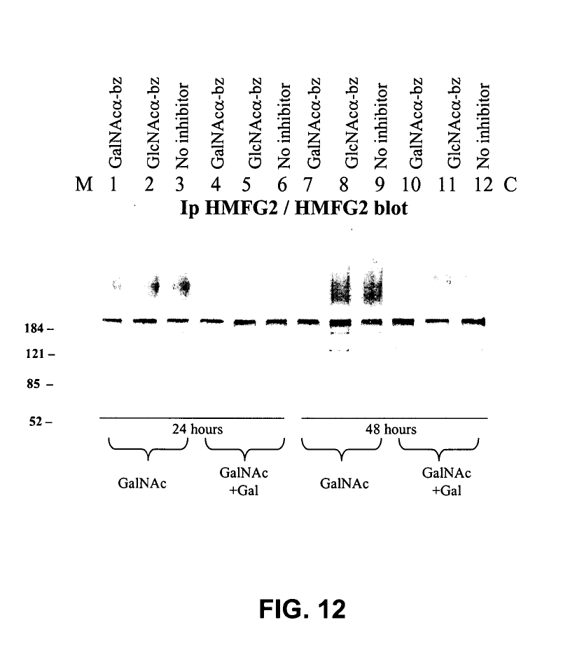

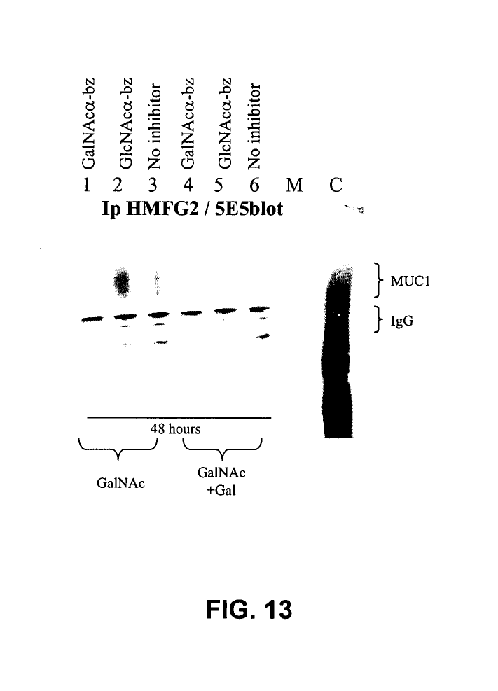

The effect of GalNAcα-benzyl on mucin transport and secretion has been concluded to be due to blockage of O-glycosylation extension⁴², FIGS. 11-13 illustrate that CHO 1d1D cells grown in Gal and GalNAc (or wild type CHO cells), and treated with 2 mM GalNAcα-benzyl in agreement with this, exhibits reduced expression of MUC1 as well as altered O-glycosylation as judged by an altered SDS-PAGE migration pattern. Wildtype CHO cells as well as CHO 1d1D cells grown in Gal and GalNAc produce O-glycans of the mono- and disialylated core 1 structures (NeuAcα2-3Galβ1-3-[NeuAcα2-6]+/−GalNAcα-O-Ser/Thr). Treatment with GalNAcα-benzyl results in some exposure of unsialylated core 1 as evaluated by staining with anti-T monoclonal antibody HH8, whereas only very little Tn is exposed as evaluated with anti-Tn monoclonal antibody 5F4. The altered SDS-PAGE migration of MUC1 produced in CHO 1d1D cells grown in Gal and GalNAc (or wild type CHO cells, not shown) shown in FIGS. 12-13 (lanes 4 and 10 when indicated) reflects mainly loss of sialic acids.

If the effect of GalNAcα-benzyl treatment on mucin transport and secretion is due to inhibition of sialylation, then treatment of CHO 1d1D cells grown only in GalNAc and hence producing only GalNAcα1-O-Ser/Thr O-glycosylation (neglible STn is produced as evidenced by lack of staining with anti-STn monoclonal antibodies 3F1 and TKH2, while cells stain very strongly with anti-Tn monoclonal antibodies 5F4 and 1E3), should have no effect on expression of MUC1 in these cells. Surprisingly as shown in FIGS. 12-13 (lanes 1 and 7 when indicated), GalNAcα-benzyl treatment does inhibit MUC1 expression in CHO 1d1D cells with O-glycosylation controlled and limited to Tn glycoforms. This result shows for the first time that mucin transport and secretion may be directly affected by treatment with GalNAcα-benzyl and not through a mechanism involving inhibition of sialylation or the O-glycosylation extension pathways. Combined with the findings of Altchuler et al.⁴¹, these results indicate that mucin transport and secretion requires some degree of GalNAc O-glycosylation, whereas O-glycan extension including sialylation seems to be of less importance for this process.

An appropriate control for GalNAcα-benzyl treatment has not previously been studied. Selection of a benzyl monosaccharide that is not involved in and does not affect glycosylation pathways in cells is problematic. We chose to use GlcNAcα-benzyl as such a control as this structure is not used in glycosylation pathways of mammalian glycoproteins and glycosphingolipids. As shown in FIGS. 12-13 (lanes 2, 5, 8, and 11, when indicated) treatment of CHO 1d1D cells with 2 mM GlcNAcα-benzyl had no effect on MUC1 expression and glycosylation. GlcNAcα-benzyl thus serves as a control for treatment of cells with benzyl sugars, and this is important because benzyl sugars and their biosynthetic products appear to aggregate in cells and cause morphological changes with prolonged treatment²⁶.

Since transport of mucin in cells was selectively inhibited by GalNAcα-benzyl (and not GlcNAcα-benzyl), even in cells limited to GalNAcα1-O-Ser/Thr O-glycosylation, we hypothesized that polypeptide GalNAc-transferases and in particular their lectin domains could be involved in ensuring mucin transport and preventing direction to lysosomes. On the one hand, GlcNAcα-benzyl does not in general inhibit GalNAc O-glycosylation and polypeptide GalNAc-transferase enzyme activity. On the other, we had previously discovered that the GalNAc-glycopeptide acceptor substrate specificities of some GalNAc-transferase isoforms including GalNAc-T4 and -T7² (PCT WO 01/85215), which suggested that these enzymes could be involved. These precepts thus give rise to plural hypothesis. One hypothesis would suggest that inhibition of the GalNAc-glycopeptide acceptor substrate specificity of GalNAc-transferases leads to mucin glycoforms with lower density of O-glycan occupancy (shown in vitro for e.g. MUC1 tandem repeats²), and that this decrease in O-glycan density results in increased targeting to lysosomal degradation and hence decrease in expression. Another hypothesis would suggest that the lectin domains of GalNAc-transferases in general have the capacity to bind GalNAc and hence provide a lectin mediated chaperone-like function, which is required for Golgi transport of O-glycosylated proteins. Lectin chaperones are well known to function ER transport as well as in lysosomal targeting⁴³, but the existence of such lectin chaperones for cell surface expression and secretion have not been demonstrated in the Golgi or trans-Golgi network.

As described above we found in the present invention that GalNAc-transferase lectins in a binding assay to GalNAc-glycopeptides surprisingly showed similar inhibition with GalNAcα-benzyl and GalNAcβ-benzyl. This indicates that these lectins in contrast to many lectins including Helix Pomatia fail to distinguish the anomeric configuration of the monosaccharide hapten recognized. βGalNAc is a rare linkage in mammalian glycoproteins and is found only in N-linked glycoproteins in man associated with the hormone specific glycosylation pattern where it generally is sulphated. Although, βGalNAc is found in both ganglioseries (GalNAcβ1-4Galβ1-4Glcβ1-Cer) and globoseries (GalNAcβ1-3Galβ1-4Galβ1-4Glcβ1-Cer) it is expected that treatment of cells with 2 mM GalNAcβ-benzyl will not interfere significantly with glycosylation. This is based on the findings that the β1,3galactosyltransferases (β3Gal-T4 and β3Gal-T5, respectively) involved in extending βGalNAc in these two glycolipid structures show no or very poor activity with GalNAcβ-benzyl^{44,45}. The only βGalNAc containing structure in O-linked glycosylation is found in the blood group related Sda structure (GalNAcβ1-4(Neuα2-3)Galβ1-3GalNAcα1-O-Ser/Thr) which has very restricted expression⁴⁶.

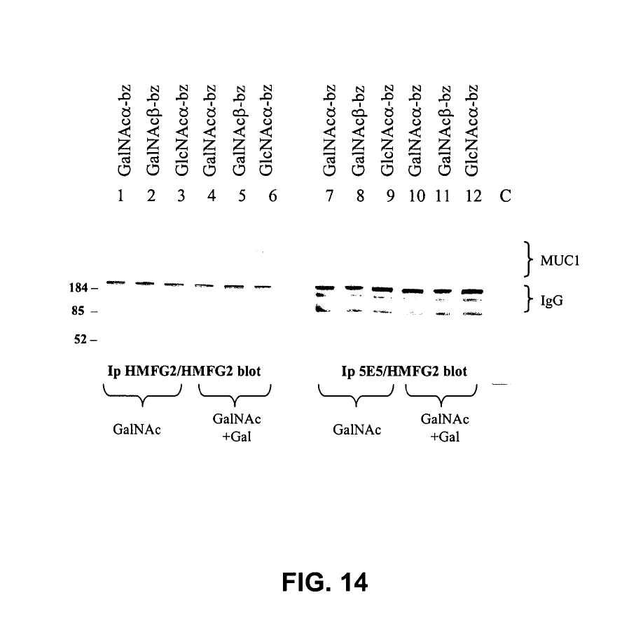

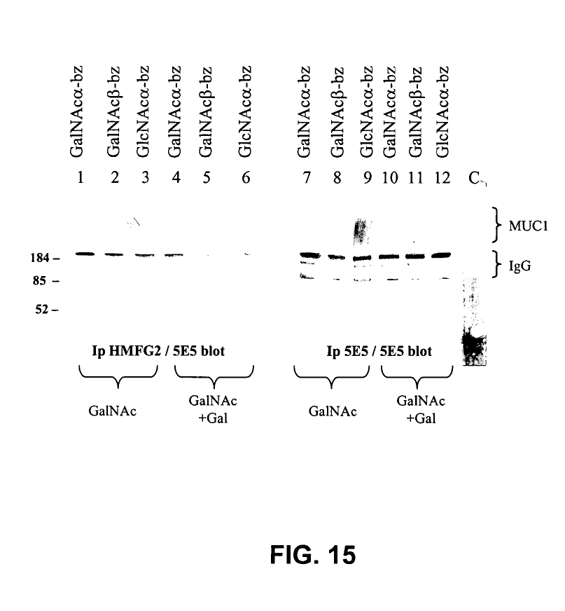

We therefore tested if GalNAcβ-benzyl treatment of cells showed the same effects as GalNAcα-benzyl treatment. As shown in FIGS. 14-15 (lane 5) GalNAcβ-benzyl treatment does not interfere with O-glycosylation in contrast to GalNAcα-benzyl (lane 4), as no difference in SDS-PAGE migration is observed. However, GalNAcα-benzyl, as well as GalNAcβ-benzyl treatment of cells, produces similar significant reduction in expression of MUC1 FIGS. 14-15. This shows surprisingly and for the first time that GalNAcβ-benzyl represents a selective inhibitor of mucin transport and secretion. GalNAcβ-benzyl is a novel preferred inhibitor of transport, surface expression and secretion of O-glycosylated proteins and mucins, because it does not interfere with the O-glycosylation extension process. GalNAcβ-benzyl further does not accumulate biosynthetic oligosaccharide products similar to those found with GalNAcα-benzyl treatment (see Example 8)²⁶. The finding that GalNAcβ-benzyl exerts these effects on mucin expression combined with the finding that it inhibits polypeptide GalNAc-transferases strongly indicate that the mechanism by which GalNAcα- and GalNAcβ-benzyl inhibits mucin expression is through inhibition of GalNAc-transferase lectins. This supports the second hypothesis articulated above. Polypeptide GalNAc-transferase lectins thus represent prime targets for intervention with mucin secretion and cell surface expression, and GalNAcβ-benzyl represents a novel selective prototype inhibitor for such intervention.

Preferred compounds for inhibition of GalNAc-transferase lectins are inactive as acceptor substrates for glycosyltransferases. In particular, the following glycosyltransferase activities: core 1 UDP-Gal:GalNAc-peptide β1,3galactosyltransferases, CMP-NeuAc:GalNAc-peptide α2,6sialyltransferases, and UDP-GlcNAc:β1,3N-acetylglucosaminyltransferases involved in O-glycosylation, are inactive with the preferred inhibitory compounds. Examples of such inhibitory compounds are GalNAcα1-O-benzyl with substitution of hydroxyl groups at C3 and/or C6 with methyl or acetyl groups to block acceptor sites.

The methods described herein are designed to identify substances and compounds that bind to and or modulate the biological activity of a polypeptide GalNAc-transferase lectin, including substances that interfere with or enhance the activity of a polypeptide GalNAc-transferase lectin.

GalNAc-transferase lectins may be used in the form of a truncated lectin domain as shown in Example 3, as a secreted GalNAc-transferase enzyme as shown in Example 2, or as a truncated protein or fusion protein with or without catalytic activity but with retained lectin domain and carbohydrate binding activity.

Agents that modulate a polypeptide GalNAc-transferase lectin can be identified based on their ability to associate with such a lectin. Therefore, the invention also provides a method of identifying agents that associate with a polypeptide GalNAc-transferase lectin. Agents identified using the method of the invention may be isolated, cloned and sequenced using conventional techniques. An agent that associates with a polypeptide GalNAc-transferase lectin may be an agonist or antagonist of the biological or immunological activity of the lectin.

Agents that can associate with a polypeptide GalNAc-transferase lectin may be identified by reacting such GalNAc-transferase lectin with a test substance, which potentially associates with a polypeptide GalNAc-transferase lectin under conditions which permit the association, and removing and/or detecting the associated GalNAc-transferase lectin and substance. The substance-lectin complex, free substance, or non-complexed lectin may be assayed. Conditions, which permit the formation of substance-lectin complexes, may be selected having regard to factors such as the nature and amounts of the substance and the lectin.

The substance-lectin complex, free substance or non-complexed lectin may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, a labelled antibody against the transferase or the substance or a labelled lectin or a labelled substance may be utilized. The antibodies, lectins, or test substances may be labelled with a detectable substance as described above.

A polypeptide GalNAc-transferase lectin, or a test substance used in the method of the invention may be insolubilized. For example, a lectin, or a test substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized lectin or substance may be prepared by reacting the material with a suitable insoluble carrier using known chemical or physical methods, for example, cyanogen bromide coupling.

The invention also contemplates a method for evaluating an agent for its ability to modulate the biological activity of a polypeptide GalNAc-transferase lectin by assaying for an agonist or antagonist (i.e. enhancer or inhibitor) of the association of the lectin with a substance that interacts with the polypeptide (e.g. carbohydrate binding site or parts thereof). The basic method for evaluating whether an agent is an agonist or antagonist of the association of a polypeptide GalNAc-transferase lectin and a substance that associates with the lectin is to prepare a reaction mixture containing the lectin and the substance under conditions which permit the formation of substance-lectin complexes, in the presence of a test agent. The test agent may be initially added to the mixture, or may be added subsequent to the addition of the lectin and substance. Control reaction mixtures without the test agent or with a placebo are also prepared. The formation of complexes is detected and the formation of complexes in the control reaction, but not in the reaction mixture, indicates that the test agent interferes with the interaction of the lectin and substance. The reactions may be carried out in the liquid phase or the lectin, substance, or test agent may be immobilized as described herein.

It will be understood that the agonists and antagonists, i.e. enhancers and inhibitors, that can be assayed using the methods of the invention may act on one or more of the interaction sites on the lectin or substance including agonist binding sites, competitive antagonist binding sites, non-competitive antagonist binding sites or allosteric sites. It will also be understood that competitive assays, in addition to direct assays, can be used to screen for and identify the agents of the present invention.

The invention also makes it possible to screen for antagonists that inhibit the effects of an agonist of the interaction of a polypeptide GalNAc-transferase lectin with a substance capable of associating with the lectin. Thus, the invention may be used to assay for an agent that competes for the same interacting site of a polypeptide GalNAc-transferase lectin.

Test compounds are screened from, for example, large libraries of synthetic or natural compounds. Numerous means are currently used for random and directed synthesis of saccharide, peptide, and nucleic acid based compounds. Examples of available libraries are synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.).

Agents which are effective in modulating a polypeptide GalNAc-transferase lectin can be identified, based on their ability to interfere with or enhance the lectin mediated binding capacity of the GalNAc-transferase protein or fragment hereof containing the lectin region. Therefore, the invention provides a method for evaluating a test substance for its ability to modulate the binding capacity of a polypeptide GalNAc-transferase lectin comprising
(a) reacting a binding substrate with a GalNAc-transferase or lectin polypeptide or fragment hereof in the presence of a test substance;
(b) measuring the amount of binding substrate bound to the GalNAc-transferase polypeptide or fragment hereof, and
(c) carrying out steps (a) and (b) in the absence of the test substance to determine if the substance interferes with or enhances the binding by the polypeptide GalNAc-transferase.

Suitable binding substrates for use in the methods of the invention are polypeptides, glycopolypeptides, or glycoproteins, which are either synthetic or naturally occurring structures. The GalNAc-transferase lectin polypeptide may be obtained from natural sources or produced using recombinant methods as described and referenced herein.

The binding or modifying substrates may be labelled with a detectable substance as described herein, and the interaction of the polypeptide of the invention with the binding or modifying substrates will give rise to a detectable change. The detectable change may be colorimetric, photometric, radiometric, potentiometric, etc. The GalNAc-transferase lectin polypeptide is reacted with the binding or modifying substrates at a pH and temperature effective for the polypeptide to bind the substrates, and where preferably one of the components is labeled, to produce a detectable change. It is preferred to use a buffer with the substrates to maintain the pH within the pH range effective for the polypeptides. The buffer and substrates may be used as an assay composition. Other compounds such as EDTA and detergents may be added to the assay composition.

The reagents suitable for applying the methods of the invention to evaluate agents that modulate a polypeptide GalNAc-transferase lectin may be packaged into convenient kits providing the necessary materials packaged into suitable containers. The kits may also include suitable supports useful in performing the methods of the invention.

Agents that modulate polypeptide GalNAc-transferase lectin(s) can also be identified by treating immortalized cells which express the transferase(s) with a test substance, and comparing the intracellular transport, degradation, surface expression, or secretion of O-glycosylated proteins, mucins, and glycoproteins performed of the cells with those of the cells in the absence of the test substance and/or with immortalized cells which do not express the transferase(s). Examples of immortalized cells that can be used include human cell lines, Chinese hamster ovary (CHO) cells and mutant cells CHO 1d1D⁴⁰, which express polypeptide GalNAc-transferase(s) or lectin(s) and produce cell membrane bound or secreted forms of the human mucin MUC1. In the absence of an inhibitor the cells will produce and transport MUC1 to the cell surface or secrete MUC1 into the growth medium. Substances that reduce the cell surface expression or the quantity of MUC1 in the medium may be considered an inhibitor.

The agents identified by the methods described herein, may be used for modulating the binding activity of a polypeptide GalNAc-transferase lectin, and they may be used as prototype drugs in the treatment of conditions mediated by a polypeptide GalNAc-transferase lectin and in designing further substances effective to treat such conditions. In particular, they may be used to alter density of O-glycosylation on glycoproteins and mucins produced by cells, the intracellular transport and surface expression of glycoproteins and mucins, the secretion of glycoproteins and mucins, and other functions governed by the polypeptide GalNAc-transferases and their lectins in transport and secretion of glycoproteins and mucins.

Therefore, the present invention has potential application in the treatment of various disorders associated with aberrant O-glycosylation and/or mucin production in mammals, preferably humans. Such disorders include the following: tumors and cancers, lungs diseases associated with mucous accumulation such as asthma, chronic bronchitis, smoker's lung, cystic fibrosis, diseases of exocrine glands associated with increased or decreased mucin secretion such as Sjøgrens syndrome, dry mouth etc. Other disorders include dysregulation of selectin-mediated leukocyte trafficking and would include but not be limited to disorders involving autoimmunity, arthritis, leukaemia's, lymphomas, immunosuppression, sepsis, wound healing, acute and chronic in action, cell mediated immunity, and the like.

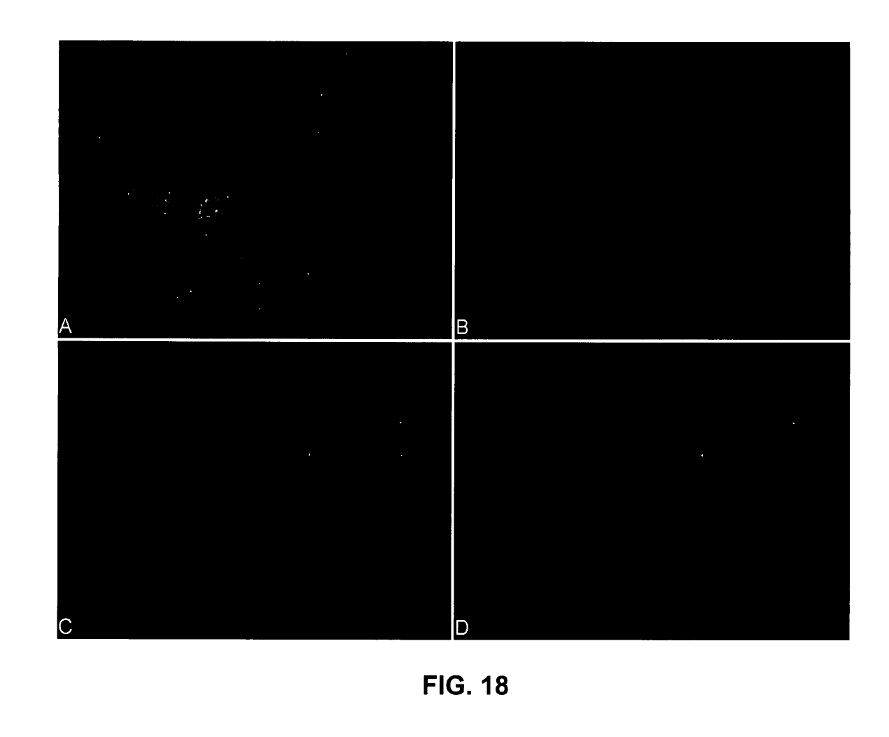

In one embodiment, the invention provides inhibitors of the secretion of mucin or O-glycosylation products, which inhibitors do not induce intracellular accumulation of vesicles containing the product, as observed in, e.g., storage diseases or conditions associated with defects in lysosomal degradation. Screening methods to identify such inhibitors are also provided. While GalNAcα-benzyl is capable of inhibiting O-glycosylation and mucin secretion, it is also associated with intracellular build-up of vesicles with the product^{60,61}. See FIG. 18. The invention further provides agents capable of preventing or treating disorders associated with aberrant mucin production and/or secretion in mammals, preferably humans, which agents inhibit secretion of one or more mucins without the disadvantage of extensive intracellular accumulation of vesicles containing the mucin. Exemplary disorders are listed above. This embodiment is based, in part, on the observation that GalNAcβ3-benzyl, in contrast to GalNAcα-benzyl, does not accumulate biosynthetic oligosaccharide products intracellularly and, as described in Example 8, blocks MUC5AC secretion without leading to intracellular vesicle accumulation or storage disease phenotype in HT-29 cells. Thus, according to the present invention, GalNAcβ-benzyl, as well as variants and derivatives thereof that also possess this functional characteristic, can be used to inhibit secretion of MUC5AC or other mucins without leading to intravesicular accumulation of the mucin in any mucin-producing and/or -secreting cell (in particular a mammalian, preferably human, cell) treated in vivo or in vitro with the GalNAcβ-benzyl compound or derivatives thereof.

The agents identified by the methods described herein, have potential application in treatment of tumors including inhibition of tumor metastasis and growth and/or regression of same. Tumor metastasis may be inhibited by inhibiting the adhesion of circulating cancer cells. The agents of the invention have particular potential application may be especially useful in the treatment of various forms of neoplasia such as leukaemias, lymphomas, melanomas, adenomas, sarcomas, and carcinomas of solid tissues in patients. In particular the composition may be used for treating malignant melanoma, pancreatic cancer, cervico-uterine cancer, cancer of the liver, kidney, stomach, lung, rectum, breast, bowel, gastric, thyroid, neck, cervix, salivary gland, bile duct, pelvis, mediastinum, urethra, bronchogenic, bladder, esophagus and colon, and Kaposi's Sarcoma which is a form of cancer associated with HIV-infected patients with Acquired Immune Deficiency Syndrome (AIDS). The substances etc. are particularly useful in the prevention and treatment of tumors of lining mucosa and glands and the metastases derived from these tumors.

Accordingly, the various agents may be formulated into pharmaceutical compositions for administration to subjects in a biologically compatible form suitable for administration in vivo. By biologically compatible form suitable for administration in vivo is meant a form of the agent to be administered in which any toxic effects are outweighed by the therapeutic effects. The agents may be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of an agent may vary according to factors such as the disease state, age, sex, and weight of the individual, and the ability of pharmaceutical composition or polypeptide to elicit a desired response in the individual. Dosage regima may be adjusted to provide the optimum therapeutic response. For example, several divided doses may be administered daily or the dose may be proportionally reduced as indicated by the exigencies of the therapeutic situation.

The active agent may be administered in a convenient manner such as by injection (subcutaneous, intravenous, etc.), oral administration, inhalation, transdermal application, or rectal administration. Depending on the route of administration, the active agent may be coated in a material to protect the agent from the action of enzymes, acids and other natural conditions that may inactivate it.

The compositions described herein can be prepared by methods known per se for the preparation of pharmaceutically acceptable compositions which can be administered to subjects, such that an effective quantity of the active agent is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the agents in association with one or more pharmaceutically acceptable vehicles or diluents, and contained in buffered solutions with a suitable pH and iso-osmotic with the physiological fluids.

The phrase “pharmaceutically acceptable” refers to molecular entities and compositions that are physiologically tolerable and do not typically produce an allergic or similar untoward reaction (for example, gastric upset, dizziness and the like) when administered to an individual. Preferably, and particularly where a immunogenic composition is used in humans, the term “pharmaceutically acceptable” denotes approved by a regulatory agency (for example, the U.S. Food and Drug Agency) or listed in a generally recognized pharmacopeia for use in animals (for example, the U.S. Pharmacopeia).

Toxicity and therapeutic efficacy of compounds can be determined by standard pharmaceutical procedures, for example in cell culture assays or using experimental animals to determine the LD50 and the ED50. The parameters LD50 and ED50 are well known in the art, and refer to the doses of a compound that are lethal to 50% of a population and therapeutically effective in 50% of a population, respectively. The dose ratio between toxic and therapeutic effects is referred to as the therapeutic index and may be expressed as the ratio: LD50/ED50. Compounds that exhibit large therapeutic indices are preferred. While compounds that exhibit toxic side effects may be used. However, in such instances it is particularly preferable to use delivery systems that specifically target such compounds to the site of affected tissue so as to minimize potential damage to other cells, tissues or organs and to reduce side effects.

Data obtained from cell culture assay or animal studies may be used to formulate a range of dosages for use in humans. The dosage of compounds used in therapeutic methods of the present invention preferably lie within a range of circulating concentrations that includes the ED50 concentration but with little or no toxicity (e.g., below the LD50 concentration). The particular dosage used in any application may vary within this range, depending upon factors such as the particular dosage form employed, the route of administration utilized, the conditions of the individual (e.g., patient), and so forth.

Non-human animals include, without limitation, laboratory animals such as mice, rats, rabbits, hamsters, guinea pigs, etc.; domestic animals such as dogs and cats; farm animals such as sheep, goats, pigs, horses, and cows; and non-human primates.

A therapeutically effective dose may be initially estimated from cell culture assays and formulated in animal models to achieve a circulating concentration range that includes the IC50. The IC50 concentration of a compound is the concentration that achieves a half-maximal inhibition of symptoms (e.g., as determined from the cell culture assays). Appropriate dosages for use in a particular individual, for example in human patients, may then be more accurately determined using such information.

Measures of compounds in plasma may be routinely measured in an individual such as a patient by techniques such as high performance liquid chromatography (HPLC) or gas chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by the routes described above.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

After pharmaceutical compositions have been prepared, they can be placed in an appropriate container and labelled for treatment of an indicated condition. For administration of an inhibitor of a polypeptide GalNAc-transferase, such labelling would include amount, frequency, and method of administration.

The use of inhibitors of the lectin domain mediated activities of the above mentioned polypeptide GalNAc-transferase isoforms and other isoforms allows for unique selective inhibition of these functions in vitro and in vivo in cells and organisms. This is desirable in manipulating the density of O-glycans, e.g. changing high density O-glycosylated tumour-associated MUC1 to low density normal MUC1 in cells. Further this is desirable in inhibiting any adhesive role the lectin domains may play in Golgi transport and intracellular sorting.

### EXAMPLES

### 1. Cloning, Expression, and Purification of Soluble GalNAc-Transferase Proteins and Soluble GalNAc-Transferase Lectins

Polypeptide GalNAc-transferases are highly conserved throughout evolution. Orthologous relationships can be defined from man to Drosophila, 48 and ortholgous members of all human polypeptide GalNAc-transferase isoforms are clearly identifiable in mouse and rats, and likely all mammals.

Polypeptide GalNAc-transferases are predicted to be type II transmembrane Golgi-resident proteins with a domain structure depicted in FIG. 12. The N-terminal cytoplasmic tail, the hydrophobic transmembrane signal sequence, and the stem region may be involved in directing Golgi-localization 47. The catalytic unit of the enzymes is approximately 300-350 amino acid residues and highly conserved in primary sequence among isoforms and also throughout evolution of the gene family 3, 48. The C-terminal region of approximately 130 amino acids exhibits similarity with the galactose binding lectin, ricin. This region show little sequence similarity among isoforms and is poorly conserved in evolution 3.

Soluble, secreted expression constructs of human GalNAc-transferases GalNAc-T1, -T2, -T3, -T4, -T6, -T7, and -T11 for baculo-virus mediated expression in insect cells have been described in detail previously^{3,8,10,32-34}. His-tagged soluble expression constructs for all human ppGalNAc-transferases, including novel genes designated GalNAc-T12, -T13, -T14, -T15, and -T16, were prepared using PCR primers as listed in Table I below.

TABLE I

Primers used for PCR of s luble secreted
GalNAc-transferase expression constructs.

GalNAc-T1:
EBHC121H:
5′ -GCGGGATCCAGGACTTCCTGCTGGAGATG- 3′
EBHC107B:
5′ -GCGGATCCTCAGAATATTTCTGGAAGGG- 3′
GalNAc-T2:
EBHC75D:
5′ -GCGGAATTCTTAAAAAGAAAGACCTTCATCACAGC- 3′
EBHC68:
5′ -GCGGAATTCCTACTGCTGCAGGTTGAGC- 3′
GalNAc-T3:
EBHC219H:
5′ -GCGGGATCCAACGATGCAAAGGAACATG- 3′
EBHC215:
5′ -AGCGGATCCAGGAACACTTAATCATTTTGGC- 3′
GalNAc-T4:
EBHC318:
5′ -GCGGGATCCTTTTCATGCCTCCGCAGGAGCC- 3′
EBHC307:
5′ -GCGGGATCCGACGAAAGTGCTCTTGTGCTC- 3′
GalNAc-T5:
EBHC909:
5′ -GCGGGATCCTGCTTTAACTGGAGGGCTAGAGC- 3′
EBHC907:
5′ -GCGGGATCCATCAGTTACACTTCAGGCTTC- 3′
GalNAc-T6:
EBHC514H:
5′ -GCGGGATCCCCTGGACCTCATGCTGGAGGCCATG- 3′
EBHC511N:
5′ -AGCGGATCCTGGGGATGATCTGGGTCCTAGAC- 3′
GalNAc-T7:
EBHC1122H:
5′ -GCGAAGCTTCAGGATGAGGGAAGACAGAGATG
EBHC1116H:
5′ -GCGAAGCTTCTCTCTAAACACTATGGATCTTATTC- 3′
GalNAc-T8:
EBHC1820:
5′ -GCGGGATCCTCTGAAAGAAAGTATGAAATTAGC- 3′
EBHC1821:
5′ -GCGGGATCCTCACTGGCTGTTGGTCTGACC
GalNAc-T9:
EBHC1320:
5′ -GCGGGATCCCTGCCGCCTGCAGGGCCGCTCCCAG- 3′
EBHC1321:
5′ -GCGGGATCCTCAGTGCCGTCGGTGTTTGATCC- 3′
GalNAc-T10:
EBHC2520:
5′ -GCGGGATCCCCGCGAGCGGCAGCCCGACGGC- 3′
EBHC2521:
5′ -GCGGGATCCTCAGTTCCTATTGAATTTTTC- 3′
GalNAc-T11:
EHHC629:
5′ -GCGAATTCGTGAACTGACTCAGCCACTTAAC- 3′
EBHC614:
5′ -GCGAATTCGTCTCTGTCAGACACGTGTC- 3′
GalNAc-T12:
EBHC1051:
5′ -GCGGGATCCGGCTCGGTGCTGCGGGCGCAGCG- 3′
EBHC1032:
5′ -GCGGGATCCTCATAACATGCCCTCTTTGAAGAACC- 3′
GalNAc-T13:
EBHC2000:
5′ -GCGGGATCCGATGTTGCACVVTCCCCACCACACC- 3′
EBHC2002:
5′ -GCGGGATCCTCATCGTTCATCCACAGCATTG- 3′
GalNAc-T14:
EBHC1720:
5′ -GCGGGATCCTCTGCTGCCTGCATTGAGGGCTG- 3′
EBH21721:
5′ -GCGGGATCCTCATGTGCCCAAGGTCATGTTCC- 3′
GalNAc-T15:
EBHC412:
5′ -GCGGGATCCCAAGAGGAAGTTGGAGGTGCCG- 3′
EBHC438:
5′ -GCGGGATCCCAGGGGTCCTCAAGAGCTCACC- 3′
GalNAc-T16:
EBHC1913:
5′ -GCGGGATCCCTACTACTTATGGCAGGACAACCG- 3′
EBHC1912:
5′ -GCGTCATGTGTGTGGCAACAGCTGCCACTG -3′

Expression constructs were amplified by PCR using 20 ng plasmid DNA as template. Expand High Fidelity-kit (Roche) was used as recommended by the manufacturer using an ABI2700 thermocycler (Applied Biosystems). Products were digested with EcoRI (GalNAc-T2, -T11, -T12 and -T16), BamHI (GalNAc-T1, -T3, -T4, -T5, -T6, -T8, -T9, -T10, -T13, -T14 and -T15) and HindIII (GalNAc-T7), and sub-cloned into the EcoRI or HindIII site of pBKS-HistagI or the BamHI site of pBKS-HistagII. PBKS-Histag-I and -II vectors were generated from pBluescrip (Stratagene), by inserting a fragment encoding 6×His, a thrombin cleavage site, and a T7 antibody site. pBKS-Histag-I was modified with the sequence

5′-GCGGCCGCTCTAGAACTAGTGGATCCAGCAGCCATCATCATCATCATCACAGCAGCGGCC
TGGTGCCGCGCGGCAGCCATATGGCTAGCATGACTGGTGGACAGCAAATGGGTCGCGGAATTCCGA
TATCAAGCTTATCGATACCGTCGACCTCGAG-3′.

pBKS-Histag-II was modified with the sequence:

5′- GAATTCGCGGCCGCAGCAGCCATCATCATCATCATCACAGCAGCGGCCTGGTGCCGCGCG
GCAGCCATATGGCTAGCATGACTGGTGGACAGCAAATGGATCCACTAGTTCTAGAGCGGCCGC -3′.

All construct were fully sequenced. His-tagged GalNAc-transferase pBKS-HIS-tag-I constructs were excised with NotI and XhoI (blunt-ended) or as for GalNAc-T11, with NotI and HindIII (blunt-ended) and sub-cloned into the NotI/BglII (blunt-ended) site of the pAcGP67A Baculo expression vector (Pharmingen). His-tagged GalNAc-transferase pBKS-HIS-tag-II constructs were excised with NotI and inserted into the NotI site of pAcGP67A Baculo expression vector.

The coding region for human polypeptide GalNAc-T12 has been submitted to GenBank/EBI Data Bank and assigned accession number AJ132365:

Human GalNAc-T12 DNA Sequence:

ATGTGGGGGCGCACGGCGCGGCGGCGCTGCCCGCGGGAACTGCGGCGCGGCCGGGAGGCGCTGTTGGTGCTCC
TGGCGCTACTGGCGTTGGCCGGGCTGGGCTCGGTGCTGCGGGCGCAGCGTGGGGCCGGGGCCGGGGCTGCCGA
GCCGGGACCCCCGCGCACCCCGCGCCCCGGGCGGCGCGAGCCGGTCATGCCGCGGCCGCCGGTGCCGGCGAAC
GCGCTGGGCGCGCGGGGCGAGGCGGTGCGGCTGCAGCTGCAGGGCGAGGAGCTGCGGCTGCAGGAGGAGAGCG
TGCGGCTGCACCAGATTAACATCTACCTCAGCGACCGCATCTCACTGCACCGCCGCCTGCCCGAGCGCTGGAA
CCCGCTGTGCAAAGAGAAGAAATATGATTATGATAATTTGCCCAGGACATCTGTTATCATAGCATTTTATAAT
GAAGCCTGGTCAACTCTCCTTCGGACAGTTTACAGTGTCCTTGAGACATCCCCGGATATCCTGCTAGAAGAAG
TGATCCTTGTAGATGACTACAGTGATAGAGAGCACCTGAAGGAGCGCTTGGCCAATGAGCTTTCGGGACTGCC
CAAGGTGCGCCTGATCCGCGCCAACAAGAGAGAGGGCCTGGTGCGAGCCCGGCTGCTGGGGGCGTCTGCGGCG
AGGGGCGATGTTCTGACCTTCCTGGACTGTCACTGTGAATGCCACGAAGGGTGGCTGGAGCCGCTGCTGCAGA
GGATCCATGAAGAGGAGTCGGCAGTGGTGTGCCCGGTGATTGATGTGATCGACTGGAACACCTTCGAATACCT
GGGGAACTCCGGGGAGCCCCAGATCGGCGGTTTCGACTGGAGGCTGGTGTTCACGTGGCACACAGTTCCTGAG
AGGGAGAGGATACGGATGCAATCCCCCGTCGATGTCATCAGGTCTCCAACAATGGCTGGTGGGCTGTTTGCTG
TGAGTAAGAAATATTTTGAATATCTGGGGTCTTATGATACAGGAATGGAAGTTTGGGGAGGAGAAAACCTCGA
ATTTTCCTTTAGGATCTGGCAGTGTGGTGGGGTTCTGGAAACACACCCATGTTCCCATGTTGGCCATGTTTTC
CCCAAGCAAGCTCCCTACTCCCGCAACAAGGCTCTGGCCAACAGTGTTCGTGCAGCTGAAGTATGGATGGATG
AATTTAAAGAGCTCTACTACCATCGCAACCCCCGTGCCCGCTTGGAACCTTTTGGGGATGTGACAGAGAGGAA
GCAGCTCCGGGACAAGCTCCAGTGTAAAGACTTCAAGTGGTTCTTGGAGACTGTGTATCCAGAACTGCATGTG
CCTGAGGACAGGCCTGGCTTCTTCGGGATGCTCCAGAACAAAGGACTAACAGACTACTGCTTTGACTATAACC
CTCCCGATGAAAACCAGATTGTGGGACACCAGGTCATTCTGTACCTCTGTCATGGGATGGGCCAGAATCAGTT
TTTCGAGTACACGTCCCAGAAAGAAATACGCTATAACACCCACCAGCCTGAGGGCTGCATTGCTGTGGAAGCA
GGAATGGATACCCTTATCATGCATCTCTGCGAAGAAACTGCCCCAGAGAATCAGAAGTTCATCTTGCAGGAGG
ATGGATCTTTATTTCACGAACAGTCCAAGAAATGTGTCCAGGCTGCGAGGAAGGAGTCGAGTGACAGTTTCGT
TCCACTCTTACGAGACTGCACCAACTCGGATCATCAGAAATGGTTCTTCAAAGAGCGCATGTTATGA

Human GalNAc-T12 Amino Acid Sequence:

MWGRTARRRCPRELRRGREALLVLLALLALAGLGSVLRAQRGAGAGAAEPGPPRTPRPGRREPVMP
RPPVPANALGARGEAVRLQLQGEELRLQEESVRLHQINIYLSDRISLHRRLPERWNPLCKEKKYDYDNLPRTS
VIIAFYNEAWSTLLRTVYSVLETSPDILLEEVILVDDYSDREHLKERLANELSGLPKVRLIPANKKKGLVRAR
LLGASAARGDVLTFLDCHCECHEGWLEPLLQRIHEEESAVVCPVIDVIDWNTFEYLGNSGEPQIGGFDWRLVF
TWHTVPERERIRMQSPVDVIRSPTMAGGLFAVSKKYFEYLGSYDTGMEVWGGENLEFSFRIWQCGGVLETHPC
SHVGHFSPSKLPTPRNKALANSVRAAEVWMDEFKELYYHRNPRARLEPFGDVTERKQLRDKLQCKDFKWFLET
VYPELHVPEDRPGFFGMLQNKGLTDYCFDYNPPDENQIVGHQVILYLCHGMGQNQFFEYTSQKEIRYNTHQPE
GCIAVEAGMDTLIMHLCEETAPENQKFILQEDGSLFHEQSKKCVQAARKESSDSFVPLLRDCTNSDHQKWFFK
ERML

The coding region for human polypeptide GalNAc-T13 has been submitted to GenBank/EBI Data Bank and assigned accession number AR153422:

Human GalNAc-T13 DNA Sequence:

ATGCTCCTAAGGAAGCGATACAGGCACAGACCATGCAGACTCCAGTTCCTCCTGCTGCTCCTGATG
CTGGGATGCGTCCTGATGATGGTGGCGATGTTGCACCCTCCCCACCACACCCTGCACCAGACTGTCACAGCCC
AAGCCAGCAAGCACAGCCCTGAAGCCAGGTACCGCCTGGACTTTGGGGAATCCCAGGATTGGGTACTGGAAGC
TGAGGATGAGOGTGAAGAGTACAGCCCTCTGGAGGGCCTGCCACCCTTTATCTCACTGCGGGAGGATCAGCTG
CTGGTGGCCGTGGCCTTACCCCAGGCCAGAAGGAACCAGAGCCAGGGCAGGAGAGGTGGGAGCTACCGCCTCA
TCAAGCAGCCAAGGAGGCAGGATAAGGAAGCCCCAAAGAGGGACTGGGGGGCTGATGAGGACGGGGAGGTGTC
TGAAGAAGAGGAGTTGACCCCGTTCAGCCTGGACCCACGTGGCCTCCAGGAGGCACTCAGTGCCCGCATCCCC
CTCCAGAGGGCTCTGCCCGAGGTGCGGCACCCACTGTGTCTGCAGCAGCACCCTCAGGACAGCCTGCCCACAG
CCAGCGTCATCCTCTGTTTCCATGATGAGGCCTGGTCCACTCTCCTGCGGACTGTACACAGCATCCTCGACAC
AGTGCCCAGGGCCTTCCTGAAGGAGATCATCCTCGTGGACGACCTCAGCCAGCAAGGACAACTCAAGTCTGCT
CTCAGCGAATATGTGGCCAGGCTGGAGGGGGTGAAGTTACTCAGGAGCAACAAGAGGCTGGGTGCCATCAGGG
CCCGGATGCTGGGGGCCACCAGAGCCACCGGGGATGTGCTCGTCTTCATGGATGCCCACTGCGAGTGCCACCC
AGGCTGGCTGGAGCCCCTCCTCAGCAGAATAGCTGGTGACAGGAGCCGAGTGGTATCTCCCGTGATAGATGTG
ATTGACTGGAAGACTTTCCAGTATTACCCCTCAAAGGACCTGCAGCGTGGGGTGTTGGACTGGAAGCTGGATT
TCCACTGGGAACCTTTGCCAGAGCATGTGAGGAAGGCCCTCCAGTCCCCCATAAGCCCCATCAGGAGCCCTGT
GGTGCCCGGAGAGGTGGTGGCCATGGACAGACATTACTTCCAAAACACTGGAGCGTATGACTCTCTTATGTCG
CTGCGAGGTGGTGAAAACCTCGAACTGTCTTTCAAGGCCTGGCTCTGTGGTGGCTCTGTTGAAATCCTTCCCT
GCTCTCGGGTAGGACACATCTACCAAAATCAGGATTCCCATTCCCCCCTCGACCAGGAGGCCACCCTGAGGAA
CAGGGTTCGCATTGCTGAGACCTGGCTGGGGTCATTCAAAGAAACCTTCTACAAGCATAGCCCAGAGGCCTTC
TCCTTGAGCAAGGCTGAGAAGCCAGACTGCATGGAACGCTTGCAGCTGCAAAGGAGACTGGGTTGTCGGACAT
TCCACTGGTTTCTGGCTAATGTCTACCCTGAGCTGTACCCATCTGAACCCAGGCCCAGTTTCTCTGGAAAGCT
CCACAACACTGGACTTGGGCTCTGTGCAGACTGCCAGGCAGAAGGGGACATCCTGGGCTGTCCCATGGTGTTG
GCTCCTTGCAGTGACAGCCGGCAGCAACAGTACCTGCAGCACACCAGCAGGAAGGAGATTCACTTTGGCAGCC
CACAGCACCTGTGCTTTGCTGTCAGGCAGGAGCAGGTGATTCTTCAGAACTGCACGGAGGAAGGCCTGGCCAT
CCACCAGCAGCACTGGGACTTCCAGGAGAATGGGATGATTGTCCACATTCTTTCTGGGAAATGCATGGAAGCT
GTGGTGCAAGAAAACAATAAAGATTTGTACCTGCGTCCGTGTGATGGAAAAGCCCGCCAGCAGTGGCGTTTTG
ACCAGATCAATGCTGTGGATGAACGATGA

Human GalNAc-T13 Amino Acid Sequence:

MLLRKRYRHRPCRLQFLLLLLMLGCVLMMVAMLHPPHHTLHQTVTAQASKHSPEARYRLDFGESQDWVLEA
EDEGEEYSPLEGLPPFTSLREDQLLVAVALPQARRNQSQGRRGGSYRLIKQPRRQDKEAPKRDWGADEDGE
VSEEEELTPFSLDPRGLQEALSARIPLQRALPEVRHPLCLQQHPQDSLPTASVILCFHDEAWSTLLRTVHS
ILDTVPRAFLKEIILVDDLSQQGQLKSALSEYVARLEGVKLLRSNKRLGAIRARMLGATRATGDVLVFMDA
HCECHPGWLEPLLSRIAGDRSRVVSPVIDVIDWKTFQYYPSKDLQRGVLDWKLDFHWEPLPEHVRKALQSP
ISPIRSPVVPGEVVAMDRHYFQNTGAYDSLMSLRGGENLELSFKAWLCGGSVEILPCSRVGHIYQNQDSHS
PLDQEATLRNRVRIAETWLGSFKETFYKHSPEAFSLSKAEKPDCMERLQLQRRLGCRTFHWFLANVYPELY
PSEPRPSFSGKLHNTGLGLCADCQAEGDILGCPMVLAPCSDSRQQQYLQHTSRKEIHFGSPQHLCFAVRQE
QVILQNCTEEGLAIHQQHWDFQENGMIVHILSGKCMEAVVQENNKDLYLRPCDGKARQQWRFDQINAVDER

The coding region for human polypeptide GalNAc-T14 has been submitted to GenBank/EBI Data Bank and assigned accession number AJ505991:

Human GalNAc-T14 DNA Sequence:

ATGAGGAGATTTGTCTACTGCAAGGTGGTTCTAGCCACTTCGCTGATGTGGGTTCTTGTTGATGTC
TTCTTACTGCTGTACTTCAGTGAATGTAACAAATGTGATGACAAGAAGGAGAGATCTCTGCTGCCTGCATTGA
GGGCTGTTATTTCAAGAAACCAAGAAGGGCCAGGAGAAATGGGAAAAGCTGTGTTGATTCCTAAAGATGACCA
GGAGAAAATGAAAGAGCTGITTAAAATCAATCAGTTTAACCTTATGGCCAGTGATTTGATTGCCCTTAATAGA
AGTCTGCCAGATGTAAGATTAGAAGGATGTAAGACAAAAGTCTACCCTGATGAACTTCCAAACACAAGTGTAG
TCATTGTGTTTCATAATGAAGCTTGGAGCACTCTCCTTAGAACTGTTTACAGTGTGATAAATCGTTCCCCACA
CTATCTACTCTCAGAGGTCATCTTGGTAGATGATGCCAGTGAAAGAGATTTTCTCAAGTTGACATTAGAGAAT
TACGTGAAAAATTTAGAAGTGCCAGTAAAAATTATTAGGATGGAAGAACGCTCTGGGTTAATACGTGCCCGTC
TTCGAGGAGCAGCTGCTTCAAAAGGGCAGGTCATAACTTTTCTTGATGCACACTGTGAATGCACGTTAGGATG
GCTGGAGCCTTTGCTGGCAAGAATAAAGGAAGACAGGAAAACGGTTGTCTGCCCTATCATTGATGTGATTAGT
GATGATACTTTTGAATATATGGCTGGGTCAGACATGACTTATGGGGGTTTTAACTGGAAACTGAATTTCCGCT
GGTATCCTGTTCCCCAAAGAGAAATGGACAGGAGGAAAGGAGACAGAACATTACCTGTCAGGACCCCTACTAT
GGCTGGTGGCCTATTTTCTATTGACAGAAACTACTTTGAAGAGATAGGAACTTACGATGCAGGAATGGATATC
TGGGGTGGAGAGAATCTTGAAATGTCTTTTAGGATTTGGCAATGTGGAGGCTCCTTGGAGATTGTTACTTGCT
CCCATGTTGGTCATGTTTTTCGGAAGGCAACTCCATACACTTTTCCTGGTGGCACTGGTCATGTCATCAACAA
GAACAACAGGAGACTGGCAGAAGTTTGGATGGATGAATTTAAAGATTTCTTCTACATCATATCCCCAGGTGTT
GTCAAAGTGGATTATGGAGATGTGTCAGTCAGAAAAACACTAAGAGAAAATCTGAAGTGTAAGCCCTTTTCTT
GGTACCTAGAAAACATCTATCCGGACTCCCAGATCCCAAGACGTTATTACTCACTTGGTGAGATAAGAAATGT
TGAAACCAATCAGTGTTTAGACAACATGGGCCGCAAGGAAAATGAAAAAGTGGGTATATTCAACTGTCATGGT
ATGGGAGGAAATCAGGTATTTTCTTACACTGCTGACAAAGAAATCCGAACCGATGACTTGTGCTTGGATGTTT
CTAGACTCAATGGACCTGTAATCATGTTAAAATGCCACCATATGAGAGGAAATCAGTTATGGGAATATGATGC
TGAGAGACTCACGTTGCGACATGTTAACAGTAACCAATGTCTCGATGAACCTTCTGAAGAAGACAAAATGGTG
CCTACAATGCAGGACTGTAGTGGAAGCAGATCCCAACAGTGGCTGCTAAGGAACATGACCTTGGGCACATGA

Human GalNAc-T14 Amino Acid Sequence:

MRRFVYCKVVLATSLMWVLVDVFLLLYFSECNKCDDKKERSLLPALRAVISRNQEGPGEMGKAVLI
PKDDQEKMKELFKINQFNLMASDLIALNRSLPDVRLEGCKTKVYPDELPNTSVVIVFHNEAWSTLLRTVYSVI
NRSPHYLLSEVILVDDASERDFLKLTLENYVKNLEVPVKIIRMEERSGLIRARLRGAAASKGQVITFLDAHCE
CTLGWLEPLLARIKEDRKTVVCPIIDVISDDTFEYMAGSDMTYGGFNWKLNFRWYPVPQREMDRRKGDRTLPV
RTPTMAGGLFSIDRNYFEEIGTYDAGMDIWGGENLEMSFRIWQCGGSLEIVTCSHVGHVFRKATPYTFPGGTG
HVINKNNRRLAEVWMDEFKDFFYIISPGVVKVDYGDVSVRKTLRENLKCKPFSWYLENIYPDSQIPRRYYSLG
EIRNVETNQCLDNNGRKENEKVGIFNCHGMGGNQVFSYTADKEIRTDDLCLDVSRLNGPVIMLKCHHMRGNQL
WEYDAERLTLRHVNSNQCLDEPSEEDKMVPTMQDCSGSRSQQWLLRNMTLGT

The coding region for human polypeptide GalNAc-T15 has been submitted to GenBank/EBI Data Bank and assigned accession number Y09324:

Human GalNAc-T15 DNA Sequence:

ATGCGGCGCCTGACTCGTCGGCTGGTTCTGCCAGTCTTCGGGGTGCTCTG
GATCACGGTGCTGCTGTTCTTCTGGGTAACCAAGAGGAAGTTGGAGGTGC
CGACGGGACCTGAAGTGCAGACCCCTAAGCCTTCGGACGCTGACTGGGAC
GACCTGTGGGACCAGTTTGATGAGCGGCGGTATCTGAATGCCAAAAAGTG
GCGCGTTGGTGACGACCCCTATAAGCTGTATGCTTTCAACCAGCGGGAGA
GTGAGCGGATCTCCAGCAATCGGGCCATCCCGGACACTCGCCATCTGAGA
TGCACACTGCTGGTGTATTGCACGGACCTTCCACCCACTAGCATCATCAT
CACCTTCCACAACGAAGCCCGCTCCACGCTGCTCAGGACCATCCGCAGTG
TATTAAACCGCACCCCTACGCATCTGATCCGGGAAATCATATTAGTGGAT
GACTTCAGCAATGACCCTGATGACTGTAAACAGCTCATCAAATTGCCCAA
GGTGAAATGCTTGCGCAATAATGAACGGCAAGGTCTGGTCCGGTCCCGGA
TTCGGGGCGCTGACATCGCCCAGGGCACCACTCTGACTTTCCTCGACAGC
CACTGTGAGGTGAACAGGGACTGGCTCCAGCCTCTGTTGCACAGGGTCAA
AGAAGACTACACGCGGGTGGTGTGCCCTGTGATCGATATCATTAACCTGG
ACACCTTCACCTACATCGAGTCTGCCTCGGAGCTCAGAGGGGGGTTTGAC
TGGAGCCTCCACTTCCAGTGGGAGCAGCTCTCCCCAGAGCAGAAGCTCGG
CGCCTGGACCCCACGGAAGCCCATCAGGACTCCTATCATAGCTGGAGGGC
TCTTCGTGATCGACAAAGCTTGGTTTGATTACCTGGGGAAATATGATATG
GACATGGACATCTGGGGTGGGGAGAACTTTGAAATCTCCTTCCGAGTGTG
GATGTGCGGGGGCAGCCTAGAGATCGTCCCCTGCAGCCGAGTGGGGCACG
TCTTCCGGAAGAAGCACCCCTACGTTTTCCCTGATGGAAATGCCAACACG
TATATAAAGAACACCAAGCGGACAGCTGAAGTGTGGATGGATGAATACAA
GCAATACTATTACGCTGCCCGGCCATTCGCCCTGGAGAGGCCCTTCGGGA
ATGTTGAGAGCAGATTGGACCTGAGGAAGAATCTGCGCTGCCAGAGCTTC
AAGTGGTACCTGGAGAATATCTACCCTGAACTCAGCATCCCCAAGGAGTC
CTCCATCCAGAAGGGCAATATCCGACAGAGACAGAAGTGCCTGGAATCTC
AAAGGCAGAACAACCAAGAAACCCCAAACCTAAAGTTGAGCCCCTGTGCC
AAGGTCAAAGGCGAAGATGCAAAGTCCCAGGTATGGGCCTTCACATACAC
CCAGCAGATCCTCCAGGAGGAGCTGTGCCTGTCAGTCATCACCTTGTTCC
CTGGCGCCCCAGTGGTTCTTGTCCTTTGCAAGAATGGAGATGACCGACAG
CAATGGACCAAAACTGGTTCCCACATCGAGCACATAGCATCCCACCTCTG
CCTCGATACAGATATGTTCGGTGATGGCACCGAGAACGGCAAGGAAATCG
TCGTCAACCCATGTGAGTCCTCACTCATGAGCCAGCACTGGGACATGGTG
AGCTCTTGAGGACCCCTGCCAGAAGCAGCAAGGGCCATGGGGTGGTGCTT
CCCTGGACCAGAACAGACTGGAAACTGGGCAGCAAGCAGCCTGCAACCAC
CTCAGACATCCTGGACTGGGAGGTGGAGGCAGAGCCCCCCAGGACAGGAG
CAACTGTCTCAGGGAGGACAGAGGAAAACATCACAAGCCAATGGGGCTCA
AAGACAAATCCCACATGTTCTCAAGGCCGTTAAGTTCCAGTCCTGGCCAGT
CATCCCTGA

Human GalNAc-T15 Amino Acid Sequence:

MRRLTRRLVLPVFGVLWITVLLFFWVTKRKLEVPTGPEVQTPKPSDADWD
DLWDQFDERRYLNAKKWRVGDDPYKLYAFNQRESERISSNRAIPDTRHLR
CTLLVYCTDLPPTSIIITFHNEARSTLLRTIRSVLNRTPTHLIREIILVD
DFSNDPDDCKQLIKLPKVKCLRNNERQGLVRSRIRGADIAQGTTLTFLDS
HCEVNRDWLQPLLHRVKEDYTRVVCPVIDIINLDTFTYIESASELRGGFD
WSLHFQWEQLSPEQKLGAWTPRKPIRTPIIAGGLFVIDKAWFDYLGKYDM
DMDIWGGENFEISFRVWMCGGSLEIVPCSRVGHVFRKKHPYVFPDGNANT
YIKNTKRTAEVWMDEYKQYYYAARPFALERPFGNVESRLDLRKNLRCQSF
KWYLENIYPELSIPKESSIQKGNIRQRQKCLESQRQNNQETPNLKLSPCA
KVKGEDAKSQVWAFTYTQQILQEELCLSVITLFPGAPVVLVLCKNGDDRQ
QWTKTGSHIEHIASHLCLDTDMFGDGTENGKEIVVNPCESSLMSQHWDMV
SS

The coding region for human polypeptide GalNAc-T16 has been submitted to GenBank/EBI Data Bank and assigned accession number AJ505951:

Human GalNAc-T16 DNA Sequence:

ATGAGGAAGATCCGCGCCAATGCCATCGCCATCCTGACCGTAGCCTGGAT
CCTGGGCACTTTCTACTACTTATGGCAGGACAACCGAGCCCACGCAGCAT
CCTCCGGCGGCCGGGGCGCGCAGAGGGCAGGCAGGAGGTCGGAGCAGCTC
CGCGAGGACCGCACCATCCCGCTCATTGTGACAGGAACTCCCTCGAAAGG
CTTTGATGAGAAGGCCTACCTGTCGGCCAAGCAGCTGAAGGCTGGAGAGG
ACCCCTACAGACAGCACGCCTTCAACCAGCTGGAGAGTGACAAGCTGAGC
CCAGACCGGCCCATCCGGGACACCCGCCATTACAGCTGCCCATCTGTGTC
CTACTCCTCGGACCTGCCAGCCACCAGCGTCATCATCACCTTCCACAATG
AGGCCCGTTCCACCCTGCTGCGCACAGTGAAGAGTGTCCTGAACCGAACT
CCTGCCAACTTGATCCAGGAGATCATTTTAGTGGATGACTTCAGCTCAGA
TCCGGAAGACTGTCTACTCCTGACCAGGATCCCCAAGGTCAAGTGCCTGC
GCAATGATCGGCGGGAAGGGCTGATCCGGTCCCGAGTGCGTGGGGCGGAC
GTGGCTGCAGCTACCGTTCTCACCTTTCTGGATAGCCACTGCGAAGTGAA
CACCGAGTGGCTGCCGCCCATGCTGCAGCGGGTGAAGGAGGACCACACCC
GCGTGGTGAGTCCCATCATTGATGTCATCAGTCTGGATAATTTTGCCTAC
CTTGCAGCATCTGCTGACCTTCGTGGAGGGTTCGACTGGAGCCTGCATTT
CAAGTGGGAGCAGATCCCTCTTGAGCAGAAGATGACCCGGACAGACCCCA
CCAGGCCCATAAGGACGCCTGTCATAGCTGGAGGAATCTTCGTGATCGAC
AAGTCCTGGTTTAACCACTTGGGAAAGTATGATGCCCAGATGGACATCTG
GGGGGGAGAGAATTTTGAGCTCTCCTTCAGGGTGTGGATGTGTGGTGGCA
GTCTGGAGATCGTCCCCTGCAGCCGGGTGGGCCATGTCTTCAGGAAACGG
CACCCCTACAACTTCCCTGAGGGTAATGCCCTCACCTACATCAGGAATAC
TAAGCGCACTGCAGAAGTGTGGATGGATGAATACAAGCAATACTACTATG
AGGCCCGGCCCTCGGCCATCGGGAAGGCCTTCGGCAGTGTGGCTACGCGG
ATAGAGCAGAGGAAGAAGATGAACTGCAAGTCCTTCCGCTGGTACCTGGA
GAACGTCTACCCAGAGCTCACGGTCCCCGTGAAGGAAGCACTCCCCGGCA
TCATTAAGCAGGGGGTGAACTGCTTAGAATCTCAGGGCCAGAACACAGCT
GGTGACTTCCTGCTTGGAATGGGGATCTGCAGAGGGTCTGCCAAGAACCC
GCAGCCCGCCCAGGCATGGCTGTTCAGTGACCACCTCATCCAGCAGCAGG
GGAAGTGCCTGGCTGCCACCTCCACCTTAATGTCCTCCCCTGGATCCCCA
GTCATACTGCAGATGTGCAACCCTAGAGAAGGCAAGCAGAAATGGAGGAG
AAAAGGATCTTTCATCCAGCATTCAGTCAGTGGCCTCTGCCTGGAGACAA
AGCCTGCCCAGCTGGTGACCAGCAAGTGTCAGGCTGACGCCCAGGCCCAG
CAGTGGCAGCTGTTGCCACACACATGA

Human GalNAc-T16 Amino Acid Sequence:

MRKIRANAIAILTVAWILGTFYYLWQDNRAHAASSGGRGAQRAGRRSEQL
REDRTIPLIVTGTPSKGFDEKAYLSAKQLKAGEDPYRQHAFNQLESDKLS
PDRPIRDTRHYSCPSVSYSSDLPATSVIITFHNEARSTLLRTVKSVLNRT
PANLIQEIILVDDFSSDPEDCLLLTRIPKVKCLRNDRREGLIRSRVRGAD
VAAATVLTFLDSHCEVNTEWLPPMLQRVKEDHTRVVSPIIDVISLDNFAY
LAASADLRGGFDWSLHFKWEQIPLEQKMTRTDPTRPIRTPVIAGGIFVID
KSWFNHLGKYDAQMDIWGGENFELSFRVWMCGGSLEIVPCSRVGHVFRKR
HPYNFFPEGNALTYTRNTKRTAEVWMDEYKQYYYEARPSAIGKAFGSVAT
RIEQRKKMNCKSFRWYLENVYPELTVPVKEALPGIIKQGVNCLESQGQNT
AGDFLLGMGICRGSAKNPQPAQAWLFSDHLIQQQGKCLAATSTLMSSPGS
PVILQMCNPREGKQKWRRKGSFIQHSVSGLCLETKPAQLVTSKCQADAQA
QQWQLLPHT

Additional homologous polypeptide GalNAc-transferase genes have been identified and cloning and expression are in progress, and it follows from the descriptions that similar methods as outlined above will yield soluble secreted proteins for study. Expression constructs may have immunoaffinity tags or purification tags at the N-terminal and/or C-terminal region. These may include myc, FLAG, HIS, GST, and other (Stratagene, Qiagen, Amersham Biosciences).

Soluble secreted expression constructs of GalNAc-transferase lectin domains were prepared from the GalNAc-transferase expression constructs described above by PCR using primer pairs as listed Table II below.

TABLE II

Primers used for amplification of
GalNAc-transferase lectin domains

GalNAc-T1 lectin domain:

| | |
|---|---|
| T1LECFOR: | 5′-CAAAGGAAGCTTATGGAGATATATCGTCAAGAG-3′ |
| T1LECREV: | 5′-GCAAGCTCGAGGCGGCCGCTCAGAATATTTCTGGAAGGGTGAC-3′ |

GalNAc-T2 lectin domain:

| | |
|---|---|
| T2LECFOR: | 5′-CAAGGAAGCTTCTTATGGAAATATTCAGAGCAGATTG-3′ |
| T2LECREV: | 5′-GCAAGCTCGAGGCGGCCGCCTACTGCTGCAGGTTGAGC-3′ |

GalNAc-T3 lectin domain:

| | |
|---|---|
| T3LECFOR: | 5′-CAAGGAAGCTTCATTTGGTGATCTTTCAAAAAGATTT-3′ |
| T3LECREV: | 5′-GCAAGCTCGAGGCGGCCGCAGGAACACTTAATCATTTTGG-3′ |

GalNAc-T4 lectin domain:

| | |
|---|---|
| T4LECFOR: | 5′-AGAAAAGAAGCTTATGGTGATATTTCTG-3′ |
| EBHC307: | 5′-AGCGGATCCGACGAAGTGCTGTTGTGCT-3′ |

GalNAc-T5 lectin domain:

| | |
|---|---|
| T5LECFOR: | 5′-CAAGGAAGCTTTAGATGTTGGCAACCTCACCCAGC-3′ |
| T5LECREV: | 5′-GCAAGCTCGAGGCGGCCGCAAGCATCAGTTACACTTCAGGCTTC-3′ |

GalNAc-T6 lectin domain:

| | |
|---|---|
| T6LECFOR: | 5′-CAAGGAAGCTTCCTTCGGTGACATTTCGGAACG-3- |
| T6LECREV: | 5′-GCAAGCTCGAGGCGGCCGCTGGGTCCTAGACAAAGAGCC-3′ |

GalNAc-T7 lectin domain:

| | |
|---|---|
| T7LECFOR: | 5′-AGAAAAGAAGCTTATGGGGATATATCGGAGCTG-3′ |
| T7LECREV: | 5′-GCAAGCTCGAGGCGGCCGCTCTCTAAACACTATGGATGTTATTC-3′ |

GalNAc-T8 lectin domain:

| | |
|---|---|
| T8LECFOR: | 5′-CAAGGAAGCTTTTGGAGACGTTTCTTCCAGAATG-3′ |
| T8LECREV: | 5′-GCAAGCTCGAGGCGGCCGCTCACTGGCTGTTGGTCTGACCCC-3′ |

GalNAc-T9 lectin domain:

| | |
|---|---|
| T9LECFOR: | 5′-CAAGGAAGCTTTCGGGGACGTGTCTGAGAGGCTG-3′ |
| T9LECREV | 5′-GCAACTCTCGAGGCGGCCGCTCAGTGCCGTGCGTGTTTGATCC-3′ |

GalNAc-T10 lectin domain:

| | |
|---|---|
| T10LECFOR: | 5′-CAAGGAAGCTTCCGCTGGGGATGTCGCAGTCCAG-3′ |
| T10LECREV: | 5′-GCAAGCTCGAGGCGGCCGCTCAGTTCCTATTGAATTTTTCC-3′ |

GalNAc-T11 lectin domain:

| | |
|---|---|
| T11LECFOR: | 5′-CAAGGAAGCTTGCAATATCAGTGAGGCTGTGG-3′ |
| T11LECREV: | 5′-GCAAGCTCGAGGCGGCCGCCCACCTTAACCTTCCAAATGC-3′ |

GalNAc-T12 lectin domain:

| | |
|---|---|
| T12LECFOR: | 5′-CAAGGAAGCTTGGGATGTGACAGAGAGGAAG-3′ |
| T12LECREV: | 5′-GCAAGCTCGAGGCGGCCGCTCATAACATGCGCTCTTTGAAGAACC-3′ |

GalNAc-T13 lectin domain:

| | |
|---|---|
| T13LECFOR: | 5′-CAAGGAAGCTTCTGAGAAGCCAGACTGCATGG-3′ |
| T13LECREV: | 5′-GCAAGCTCGAGGCGGCCGCTCATCGTTCATCCACAGCATTG-3′ |

GalNAc-T14 lectin domain:

| | |
|---|---|
| T14LECFOR: | 5′-CAAGGAAGCTTATGGAGATGTGTCAGTCAGAAAAAC-3′ |
| T14LECREV: | 5′-GCAAGCTCGAGGCGGCCGCTCATGTGCCCAAGGTCATGTTCC-3′ |

GalNAc-T15 lectin domain:

| | |
|---|---|
| T15LECFOR: | 5′-CAAGGAAGCTTTCGGGAATGTTGAGAGCAGATTG-3′ |
| T15LECREV: | 5′-GCAAGCTCGAGGCGGCCGCTCAAGAACTCACCATGTCCCAGTG-3′ |

GalNAc-T16 lectin domain:

| | |
|---|---|
| T16LECFOR: | 5′-CAAGGAAGCTTGCAGTGTGGCTACGCGGATAGAGCAGAGG-3′ |
| T16LECREV: | 5′-GCAAGCTCGAGGCGGCCGCTCATGTGTGTGGCAACAGCTGCC-3′ |

PCR amplifications were performed with 10 ng GalNAc-transferase plasmid DNA as template and High Fidelity PCR kit (Roche) with conditions recommended by the manufacturer. Amplified products were digested with HindIII and XhoI and inserted into the HindIII/XhoI site of pBKS-HistagI. All constructs were fully sequenced. Tagged lectin domain constructs were excised with NotI and sub-cloned into the NotI site of pAcGP67-A Baculo expression vector.

The exact borders of the lectin domains and the catalytic units have not been defined, but multiple sequence alignment analysis (FIG. 2) was used to predict the most likely borders and these were used for design of PCR primers as listed in Table II. DNA and amino acid sequences of preferred constructs of GalNAc-transferase lectin domains and their construct design include the following (Table III):

TABLE III

DNA and amino acid sequences of
GalNAc-transferase lectin domains.

GalNAc-T1 lectin domain:
The lectin domain polypeptide sequence comprises
amino acid residues 393-559 of GALNT1 (GALNT1
nucleotide sequence accession number is AJ505952)
T1 LECTIN DNA sequence
AAAGAAGCTTATGGAGATATATCGTCAAGAGTTGGTCTAAGACACAAACT
ACAATGCAAACCTTTTTCCTGGTACCTAGAGAATATATATCCTGATTCTC
AAATTCCACGTCACTATTTCTCATTGGGAGAGATACGAAATGTGGAAACG
AATCAGTGTCTAGATAACATGGCTAGAAAAGAGAATGAAAAAGTTGGAAT
TTTTAATTGCCATGGTATGGGGGGTAATCAGGTTTTCTCTTATACTGCCA
ACAAAGAAATTAGAACAGATGACCTTTGCTTGGATGTTTCCAAACTTAAT
GGCCCAGTTACAATGCTCAAATGCCACCACCTAAAAGGCAACCAACTCTG
GGAGTATGACCCAGTGAAATTAACCCTGCAGCATGTGAACAGTAATCAGT
GCCTGGATAAAGCCACAGAACAGGATAGCCAGGTGCCCAGCATTAGAGAC
TGCAATGGAAGTCGGTCCCAGCAGTGGCTTCTTCGAAACGTCACCCTTCC
AGAAATATTCTGA-stop
T1 LECTIN Amino acid sequence
YGDISSRVGLRHKLQCKPFSWYLENIYPDSQIPRHYFSLGEIRNVETNQC
LDNMARKENEKVGIFNCHGMGGNQVFSYTANKEIRTDDLCLDVSKLNGPV
TMLKCHHLKGNQLWEYDPVKLTLQHVNSNQCLDKATEEDSQVPSIRDCNG
SRSQQWLLRNVTLPEIF*

GalNAc-T2 Lectin Domain:

The lectin domain polypeptide sequence comprises amino acid residues 408-571 of GALNT2 (GALNT2 nucleotide sequence accession number is X85019).

T2 LECTIN DNA sequence
TATCCAGAGTTAAGGGTTCCAGACCATCAGGATATAGCTTTTGGGGCCTT
GCAGCAGGGAACTAACTGCCTCGACACTTTGGGACACTTTGCTGATGGTG
TGGTTGGAGTTTATGAATGTCACAATGCTGGGGGAAACCAGGAATGGGCC
TTGACGAAGGAGAAGTCGGTGAAGCACATGGATTTGTGCCTTACTGTGGT
GGACCGGGCACCGGGCTCTCTTATAAAGCTGCAGGGCTGCCGAGAAAATG
ACAGCAGACAGAAATGGGAACAGATCGAGGGCAACTCCAAGCTGAGGCAC
GTGGGCAGCAACCTGTGCCTGGACAGTCGCACGGCCAAGAGCGGGGGCCT
AAGCGTGGAGGTGTGTGGCCCGGCCCTTTCGCAGCAGTGGAAGTTCACGC
TCAACCTGCAGCAGTAG-stop
T2 LECTIN Amino acid sequence
YPELRVPDHQDIAFGALQQGTNCLDTLGHFADGVVGVYECHNAGGNQEWA
LTKEKSVKHMDLCLTVVDRAPGSLIKLQGCRENDSRQKWEQIEGNSKLRH
VGSNLCLDSRTAKSGGLSVEVCGPALSQQWKFTLNLQQ*

GalNAc-T3 Lectin Domain:

The lectin domain polypeptide sequence comprises amino acid residues 467-633 of GALNT3 (GALNT3 nucleotide sequence accession number is AJ505954).

T3 LECTIN DNA sequence
TCATTTGGTGATCTTTCAAAAAGATTTGAAATAAAACACCGTCTTCGGTG
TAAAAATTTTACATGGTATCTGAACAACATTTATCCAGAGGTGTATGTGC
CAGACCTTAATCCTGTTATATCTGGATACATTAAAAGCGTTGGTCAGCCT
CTATGTCTGGATGTTGGAGAAAACAATCAAGGAGGCAAACCATTAATTAT
GTATACATGTCATGGACTTGGGGGAAACCAGTACTTTGAATACTCTGCTC
AACATGAAATTCGGCACAACATCCAGAAGGAATTATGTCTTCATGCTGCT
CAAGGTCTCGTTCAGCTGAAGGCATGTACCTACAAAGGTCACAAGACAGT
TGTCACTGGAGAGCAGATATGGGAGATCCAGAAGGATCAACTTCTATACA
ATCCATTCTTAAAAATGTGCCTTTCAGCAAATGGAGAGCATCCAAGTTTA
GTGTCATGCAACCCATCAGATCCACTCCAAAAATGGATACTTAGCCAAAA
TGATTAA-stop
T3 LECTIN Amino acid sequence
FGDLSKRFEIKHRLRCKNFTWYLNNIYPEVYVPDLNPVISGYIKSVGQPL
CLDVGENNQGGKPLIMYTCHGLGGNQYFEYSAQHEIRHNIQKELCLHAAQ
GLVQLKACTYKGHKTVVTGEQIWEIQKDQLLYNPFLKMCLSANGEHPSLV
SCNPSDPLQKWILSQND*

GalNAc-T4 Lectin Domain:

The lectin domain polypeptide sequence comprises amino acid residues 405-578 of GALNT4 (GALNT4 nucleotide sequence accession number is YO8564).

T4 LECTIN DNA sequence
GAGGATAGACCAGGCTGGCATGGGGCTATTCGCAGTAGAGGGATCTCGTC
TGAATGTTTAGATTATAATTCTCCTGACAACAACCCCACAGGTGCTAACC
TTTCACTGTTTGGATGCCATGGTCAAGGAGGCAATCAATTCTTTGAATAT
ACTTCAAACAAAGAAATAAGGTTTAATTCTGTGACAGAGTTATGTGCAGA
GGTACCTGAGCAAAAAATTATGTGGGAATGCAAAATTGTCCCAAAGATGG
GTTCCCTGTACCAGCAAACATTATTTGGCATTTTAAAGAAGATGGAACTA
TTTTTCACCCACACTCAGGACTGTGTCTTAGTGCTTATCGGACACCGGAG
GGCCGACCTGATGTACAAATGAGAACTTGTGATGCTCTAGATAAAAATCA
AATTTGGAGTTTTGAGAAATAG-stop
T4 LECTIN Amino acid sequence
AYGDISERKLLRERLRCKSFDWYLKNVFPNLHVPEDRPGWHGAIRSRGIS
SECLDYNSPDNNPTGANLSLFGCHGQGGNQFFEYTSNKEIRFNSVTELCA
EVPEQKNYVGMQNCPKDGFPVPANIIWHFKEDGTIFHPHSGLCLSAYRTP
EGRPDVQMRTCDALDKNQIWSFEK*

GalNAc-T5 Lectin Domain:

The lectin domain polypeptide sequence comprises amino acid residues 486-653 of GALTN5 (GALTN5 nucleotide sequence accession number is AJ505956).

T5 LECTIN DNA sequence
TTAGATGTTGGCAACCTCACCCAGCAAAGGGAGCTGCGAAGAAACTGAAG
TGCAAJTAGTTTCAAATGGTACTTGGAGAATGTCTTTCCTGACTTAAGGG
CTCCCATTGTGAGAGCTAGTGGTGTGCTTATTAATGTGGCTTTGGGTAAA
TGCATTTCCATTGAAAACACTACAGTCATTCTGGTTTGACTGCGATGGGA
GCAAAGAGCTTCAACAATTTAATTACACCTGGTTAAGACTTATTAAATGT
GGAGAATGGTGTATAGCCCCCATCCCTGATAAAGGAGCCGTAAGGCTGCA
CCCTTGTGATAACAGAAACAAAGGGCTAAAATGGCTGCATAAATCAACAT
CAGTCTTTCATCCAGAACTGGTGAATCACATTGTTTTTGAAAACAATCAG
CAATTATTATGCTTGGAAGGAAATTTTTCTCAAAAGATCCTGAAAGTAGC
TGCCTGTGACCCAGTGAAGCCATATCAAAAGTGGAAATTTGAAAAATATT
ATGAAGCCTGA-stop
T5 LECTIN Amino acid sequence
DVGNLTQQRELRKKLKCKSFKWYLENVFPDLPAPIVRASGVLINVALGKC
ISIENTTVILEDCDGSKELQQFNYTWLRLIKCGEWCIAPIPDKGAVRLHP
CDNRNKGLKWLHKSTSVFHPELVNHIVFENNQQLLCLEGNFSQKILKVAA
CDPVKPYQKWKFEKYYEA*

GalNAc-T6 Lectin Domain:

The lectin polypeptide sequence comprises amino acid residues 458-622 of GALNT6 (GALNT6 nucleotide sequence accession number is AJ133523)

T6 LECTIN DNA sequence
TCCTTCGGTGACATTTCGGAACGACTGCAGCTGAGGGAACAACTGCACTG
TCACAACTTTTCCTGGTACCTGCACAATGTCTACCCAGAGATGTTTGTTC
CTGACCTGACGCCCACCTTCTATGGTGCCATCAAGAACCTCGGCACCAAC
CAATGCCTGGATGTGGGTGAGAACAACCGCGGGGGGAAGCCCCTCATCAT
GTACTCCTGCCACGGCCTTGGCGGCAACCAGTACTTTGAGTACACAACTC
AGAGGGACCTTCGCCACAACATCGCAAAGCAGCTGTGTCTACATGTCAGC
AAGGGTGCTCTGGGCCTTGGGAGCTGTCACTTCACTGGCAAGAATAGCCA
GGTCCCCAAGGACGAGGAATGGGAATTGGCCCAGGATCAGCTCATCAGGA
ACTCAGGATCTGGTACCTGCCTGACATCCCAGGACAAAAAGCCAGCCATG
GCCCCCTGCAATCCCAGTGACCCCCATCAGTTGTGGCTCTTTGTCTAG-
stop
T6 LECTIN Amino acid sequence
SFGDISERLQLREQLHCHNFSWYLHNVYPEMFVPDLTPTFYGAIKNLGTN
QCLDVGENNRGGKPLIMYSCHGLGGNQYFEYTTQRDLRHNIAKQLCLHVS
KGALGLGSCHFTGKNSQVPKDEEWELAQDQLIRNSGSGTCLTSQDKKPAM
APCNPSDPHQLWLFV*

GalNAc-T7 Lectin Domain:

The lectin domain polypeptide sequence comprises amino acid residues 492-657 of GALNT7 (GALNT7 nucleotide sequence accession number is AJ505958).

T7 LECTIN DNA sequence
TATGGGGATATATCGGAGCTGAAAAAATTTCGAGAAGATCACAACTGCCA
AAGTTTTAAGTGGTTCATGGAAGAAATAGCTTATGATATCACCTCACACT
ACCCTTTGCCACCCAAAAATGTTGACTGGGGAGAAATCAGAGGCTTCGAA
ACTGCTTACTGCATTGATAGCATGGGAAAAACAAATGGAGGCTTTGTTGA
ACTAGGACCCTGCCACAGGATGGGAGGGAATCAGCTTTTCAGAATCAATG
AAGCAAATCAACTCATGCAGTATGACCAGTGTTTGACAAAGGGAGCTGAT
GGATCAAAAGTTATGATTACACACTGTAATCTAAATGAATTTAAGGAATG
GCAGTACTTCAAGAACCTGCACAGATTTACTCATATTCCTTCAGGAAAGT
GTTTAGATCGCTCAGAGGTCCTGCATCAAGTATTCATCTCCAATTGTGAC
TCCAGTAAAACGACTCAAAAATGGGAAATGAATAACATCCATAGTGTTTA
G-stop
T7 LECTIN Amino acid sequence
YGDISELKKFREDHNCQSFKWFMEEIAYDITSHYPLPPKNVDWGEIRGFE
TAYCIDSMGKTNGGFVELGPCHRMGGNQLFRINEANQLMQYDQCLTKGAD
GSKVMITHCNLNEFKEWQYFKNLHRFTHIPSGKCLDRSEVLHQVFISNCD
SSKTTQKWEMNNIHSV*

GalNAc-T8 Lectin Domain:

The lectin domain polypeptide sequence comprises amino acid residues 459-637 of GALNT8 (GALNT8 nucleotide sequence accession number is AJ505959).

T8 LECTIN DNA sequence
GACGTTTCTTCCAGAATGGCACTCCGGGAAAAACTGAAATGTAAAACTTT
TGACTGGTACCTGAAAAATGTTTATCCACTCTTGAAGCCACTCCACACCA
TCGTGGGCTATGGAAGAATGAAAAACCTATTGGATGAAAATGTCTGCTTG
GATCAGGGACCCGTTCCAGGCAACACCCCCATCATGTATTACTGCCATGA
ATTCAGCTCACAGAATGTCTACTATCACCTAACTGGGGAGCTCTATGTGG
GACAACTGATTGCAGAGGCCAGTGCTAGTGATCGCTGCCTGACAGACCCT
GGCAAGGCGGAGAAGCCCACCTTAGAACCATGCTCCAAGGCAGCTAAGAA
TAGACTGCATATATATTGGGATTTTAAACCGGGAGGAGCTGTCATAAACA
GAGATACCAAGCGGTGTCTGGAGATGAAGAAGGATCTTTTGGGTAGCCAC
GTGCTTGTGCTCCAGACCTGTAGCACGCAAGTGTGGGAAATCCAGCACAC
TGTCAGAGACTGGGGTCAGACCAACAGCCAGTGA//
T8 LECTIN Amino acid sequence
FGDVSSRMALREKLKCKTFDWYLKNVYPLLKPLHTIVGYGRMKNLLDENV
CLDQGPVPGNTPIMYYCHEFSSQNVYYHLTGELYVGQLIAEASASDRCLT
DPGKAEKPTLEPCSKAAKNRLHIYWDFKPGGAVINRDTKRCLEMKKDLLG
SHVLVLQTCSTQVWEIQHTVRDWGQTNSQ//

GalNAc-T9 Lectin Domain:

The lectin domain polypeptide sequence comprises amino acid residues 427-603 of GALNT9 (GALNT9 nucleotide sequence accession number is AJ505960).

T9 LECTIN DNA sequence
TTCGGGGACGTGTCTGAGAGGCTGGCCCTGCGTCAGAGGCTGAAGTGTCG
CAGCTTCAAGTGGTACCTGGAGAACGTGTACCCGGAGATGAGGGTCTACA
ACAACACCCTCACGTACGGAGAGGTGAGAAACAGCAAAGCCAGTGCCTAC
TGTCTGGACCAGGGAGCGGAGGACGGCGACCGGGCGATCCTCTACCCCTG
CCACGGGATGTCCTCCCAGCTGGTGCGGTACAGCGCTGACGGCCTGCTGC
AGCTGGGGCCTCTGGGCTCCACAGCCTTCTTGCCTGACTCCAAGTGTCTG
GTGGATGACGGCACGGGCCGCATGCCCACCCTGAAGAGGTGTGAGGATGT
GGCGCGGCCAACACAGCGGCTGTGGGACTTCACCCAGAGTGGCCCCATTG
TGAGCCGGGCCACGGGCCGCTGCCTGGAGGTGGAGATGTCCAAAGATGCC
AACTTTGGGCTCCGGCTGGTGGTACAGAGGTGCTCGGGGCAGAAGTGGAT
GATCAGAAACTGGATCAAACACGCACGGCACTGA-stop
T9 LECTIN Amino acid sequence
FGDVSERLALRQRLKCRSFKWYLENVYPEMRVYNNTLTYGEVRNSKASAY
CLDQGAEDGDRAILYPCHGMSSQLVRYSADGLLQLGPLGSTAFLPDSKCL
VDDGTGRMPTLKRCEDVARPTQRLWDFTQSGPIVSRATGRCLEVEMSKDA
NFGLRLVVQRCSGQKWMIRNWIKHARH*

GalNAc-T10 Lectin Domain:

The lectin domain polypeptide sequence comprises amino acid residues 417-603 of GALNT10 (GALNT10 nucleotide sequence accession number is AJ505950).

T10 LECTIN DNA sequence
GCTGGGGATGTCGCAGTCCAGAAAAAGCTCCGCAGCTCCCTTAACTGCAA
GAGTTTCAAGTGGTTTATGACGAAGATAGCCTGGGACCTGCCCAAATTCT
ACCCACCCGTGGAGCCCCCGGCTGCAGCTTGGGGGGAGATCCGAAATGTG
GGCACAGGGCTGTGTGCAGACACAAAGCACGGGGCCTTGGGCTCCCCACT
AAGGCTAGAGGGCTGCGTCCGAGGCCGTGGGGAGGCTGCCTGGAACAACA
TGCAGGTATTCACCTTCACCTGGAGAGAGGACATCCGGCCTGGAGACCCC
CAGCACACCAAGAAGTTCTGCTTTGATGCCATTTCCCACACCAGCCCTGT
CACGCTGTACGACTGCCACAGCATGAAGGGCAACCAGCTGTGGAAATACC
GCAAAGACAAGACCCTGTACCACCCTGTCAGTGGCAGCTGCATGGACTGC
AGTGAAAGTGACCATAGGATCTTCATGAACACCTGCAACCCATCCTCTCT
CACCCAGCAGTGGCTGTTTGAACACACCAACTCAACAGTCTTGGAAAAAT
TCAATAGGAACTGA
T10 LECTIN Amino acid sequence
AGDVAVQKKLRSSLNCKSFKWFMTKIAWDLPKFYPPVEPPAAAWGEIRNV
GTGLCADTKHGALGSPLRLEGCVRGRGEAAWNNMQVFTFTWREDIRPGDP
QHTKKFCFDAISHTSPVTLYDCHSMKGNQLWKYRKDKTLYHPVSGSCMDC
SESDHRIFMNTCNPSSLTQQWLFEHTNSTVLEKFNRN*

GalNAc-T11 Lectin Domain:

The lectin domain polypeptide sequence comprises amino acid residues 492-608 of GALNT11 (GALNT11 nucleotide sequence accession number is Y12434).

T11 LECTIN DNA sequence
TGCAATATCAGTGAGCGTGTGGAACTGAGAAAGAAGTTGGGCTGTAAATC
ATTTAAATGGTATTTGGATAATGTATACCCAGAGATGCAGATATCTGGGT
CCCACGCCAAACCCCAACAACCCATTTTTGTCAATAGAGGGCCAAAACGA
CCCAAAGTCCTTCAACGTGGAAGGCTCTATCACCTCCAGACCAACAAATG
CCTGGTGGCCCAGGGCCGCCCAAGTCAGAAGGGAGGTCTCGTGGTGCTTA
AGGCCTGTGACTACAGTGACCCAAATCAGATCTGGATCTATAATGAAGAG
CATGAATTGGTTTTAAATAGTCTCCTTTGTCTAGATATGTCAGAGACTCG
CTCATCAGACCCGCCACGGCTCATGAAATGCCACGGGTCAGGAGGATCCC
AGCAGTGGACCTTTGGGAAAAACAATCGGCTATACCAGGTGTCGGTTGGA
CAGTGCCTGAGAGCAGTGGATCCCCTGGGTCAGAAGGGCTCTGTCGCCAT
GGCGATCTGCGATGGCTCCTCTTCACAGCAGTGGCATTTGGAAGGTTAA
T11 LECTIN Amino acid sequence
NISERVELRKKLGCKSFKWYLDNVYPEMQISGSHAKPQQPIFVNRGPKRP
KVLQRGRLYHLQTNKCLVAQGRPSQKGGLVVLKACDYSDPNQIWIYNEEH
ELVLNSLLCLDMSETRSSDPPRLMKCHGSGGSQQWTFGKNNRLYQVSVGQ
CLRAVDPLGQKGSVAMAICDGSSSQQWHLEG*

GalNAc-T12 Lectin Domain:

The lectin domain polypeptide sequence comprises amino acid residues 428-581 of GALNT12 (GALNT12 nucleotide sequence accession number is AJ505963).

T12 LECTIN DNA sequence
TGGGATGTGACAGAGAGGAAGCAGCTCCGGGACAAGCTCCAGTGTAAAGA
CTTCAAGTGGTTCTTGGAGACTGTGTATCCAGAACTGCATGTGCCTGAGG
ACAGGCCTGGCTTCTTCGGGATGCTCCAGAACAAAGGACTAACAGACTAC
TGCTTTGACTATAACCCTCCCGATGAAAACCAGATTGTGGGACACCAGGT
CATTCTGTACCTCTGTCATGGGATGGGCCAGAATCAGTTTTTCGAGTACA
CGTCCCAGAAAGAAATACGCTATAACACCCACCAGCCTGAGGGCTGCATT
GCTGTGGAAGCAGGAATGGATACCCTTATCATGCATCTCTGCGAAGAAAC
TGCCCCAGAGAATCAGAAGTTCATCTTGCAGGAGGATGGATCTTTATTTC
ACGAACAGTCCAAGAAATGTGTCCAGGCTGCGAGGAAGGAGTCGAGTGAC
AGTTTCGTTCCACTCTTACGAGACTGCACCAACTCGGATCATCAGAAATG
GTTCTTCAAAGAGCGCATGTTATGA
T12 LECTIN Amino acid sequence
DVTERKQLRDKLQCKDFKWFLETVYPELHVPEDRPGFFGMLQNKGLTDYC
FDYNPPDENQIVGHQVILYLCHGMGQNQFFEYTSQKEIRYNTHQPEGCIA
VEAGMDTLIMHLCEETAPENQKFILQEDGSLFHEQSKKCVQAARKESSDS
FVPLLRDCTNSDHQKWFFKERML*

GalNAc-T13 Lectin Domain:

The lectin domain polypeptide sequence comprises amino acid residues 466-639 of GALNT13 (GALNT13 nucleotide sequence accession number is AJ505964).

T13 LECTIN DNA sequence
TCTGAGAAGCCAGACTGCATGGAACGCTTGCAGCTGCAAAGGAGACTGGG
TTGTCGGACATTCCACTGGTTTCTGGCTAATGTCTACCCTGAGCTGTACC
CATCTGAACCCAGGCCCAGTTTCTCTGGAAAGCTCCACAACACTGGACTT
GGGCTCTGTGCAGACTGCCAGGCAGAAGGGGACATCCTGGGCTGTCCCAT
GGTGTTGGCTCCTTGCAGTGACAGCCGGCAGCAACAGTACCTGCAGCACA
CCAGCAGGAAGGAGATTCACTTTGGCAGCCCACAGCACCTGTGCTTTGCT
GTCAGGCAGGAGCAGGTGATTCTTCAGAACTGCACGGAGGAAGGCCTGGC
CATCCACCAGCAGCACTGGGACTTCCAGGAGAATGGGATGATTGTCCACA
TTCTTTCTGGGAAATGCATGGAAGCTGTGGTGCAAGAAAACAATAAAGAT
TTGTACCTGCGTCCGTGTGATGGAAAAGCCCGCCAGCAGTGGCGTTTTGA
CCAGATCAATGCTGTGGATGAACGATGA
T13 LECTIN Amino acid sequence
EKPDCMERLQLQRRLGCRTFHWFLANVYPELYPSEPRPSFSGKLHNTGLG
LCADCQAEGDILGCPMVLAPCSDSRQQQYLQHTSRKEIHFGSPQHLCFAV
RQEQVILQNCTEEGLAIHQQHWDFQENGMIVHILSGKCMEAVVQENNKDL
YLRPCDGKARQQWRFDQINAVDER*

GalNAc-T14 Lectin Domain:

The lectin domain polypeptide sequence comprises amino acid residues 352-516 of GALNT14 (GALNT14 nucleotide sequence accession number is AJ505991).

T14 LECTIN DNA sequence
TATGGAGATGTGTCAGTCAGAAAAACACTAAGAGAAAATCTGAAGTGTAA
GCCCTTTTCTTGGTACCTAGAAAACATCTATCCGGACTCCCAGATCCCAA
GACGTTATTACTCACTTGGTGAGATAAGAAATGTTGAAACCAATCAGTGT
TTAGACAACATGGGCCGCAAGGAAAATGAAAAAGTGGGTATATTCAACTG
TCATGGTATGGGAGGAAATCAGGTATTTTCTTACACTGCTGACAAAGAAA
TCCGAACCGATGACTTGTGCTTGGATGTTTCTAGACTCAATGGACCTGTA
ATCATGTTAAAATGCCACCATATGAGAGGAAATCAGTTATGGGAATATGA
TGCTGAGAGACTCACGTTGCGACATGTTAACAGTAACCAATGTCTCGATG
AACCTTCTGAAGAAGACAAAATGGTGCCTACAATGCAGGACTGTAGTGGA
AGCAGATCCCAACAGTGGCTGCTAAGGAACATGACCTTGGGCACATGA
T14 LECTIN Amino acid sequence
YGDVSVRKTLRENLKCKPFSWYLENIYPDSQIPRRYYSLGEIRNVETNQC
LDNMGRKENEKVGIFNCHGMGGNQVFSYTADKEIRTDDLCLDVSRLNGPV
IMLKCHHMRGNQLWEYDAERLTLRHVNSNQCLDEPSEEDKMVPTMQDCSG
SRSQQWLLRNMTLGT*

GalNAc-T15 Lectin Domain:

The lectin domain polypeptide sequence comprises amino acid residues 382-552 of GALNT15 (GALNT15 nucleotide sequence accession number is AJ505966).

T15 LECTIN DNA sequence
TCGGGAATGTTGAGAGCAGATTGGACCTGAGGAAGAATCTGCGCTGCCAG
AGCTTCAAGTGGTACCTGGAGAATATCTACCCTGAACTCAGCATCCCCAA
GGAGTCCTCCATCCAGAAGGGCAATATCCGACAGAGACAGAAGTGCCTGG
AATCTCAAAGGCAGAACAACCAAGAAACCCCAAACCTAAAGTTGAGCCCC
TGTGCCAAGGTCAAAGGCGAAGATGCAAAGTCCCAGGTATGGGCCTTCAC
ATACACCCAGAAGATCCTCCAGGAGGAGCTGTGCCTGTCAGTCATCACCT
TGTTCCCTGGCGCCCCAGTGGTTCTTGTCCTTTGCAAGAATGGAGATGAC
CGACAGCAATGGACCAAAACTGGTTCCCACATCGAGCACATAGCATCCCA
CCTCTGCCTCGATACAGATATGTTCGGTGATGGCACCGAGAACGGCAAGG
AAATCGGCGTCAACCCATGTGAGTCCTCACTCATGAGCCAGCACTGGGAC
ATGGTGAGTTCTTGAG
T15 LECTIN Amino acid sequence
FGNVESRLDLRKNLRCQSFKWYLENIYPELSIPKESSIQKGNIRQRQKCL
ESQRQNNQETPNLKLSPCAKVKGEDAKSQVWAFTYTQKILQEELCLSVIT
LFPGAPVVLVLCKNGDDRQQWTKTGSHIEHIASHLCLDTDMFGDGTENGK
EIGVNPCESSLMSQHWDMVSS*

GalNAc-T16 Lectin Domain:

The lectin domain polypeptide sequence comprises amino acid residues 396-558 of GALNT16 (GALNT16 nucleotide sequence accession number is AJ505951).

T16 LECTIN DNA sequence
AGTGTGGCTACGCGGATAGAGCAGAGGAAGAAGATGAACTGCAAGTCCTTCCGCTGGTACCT
GGAGAACGTCTACCCAGAGCTCACGGTCCCCGTGAAGGAAGCACTCCCCGGCATCATTAAGC
AGGGGGTGAACTGCTTAGAATCTCAGGGCCAGAACACAGCTGGTGACTTCCTGCTTGGAATG
GGGATCTGCAGAGGGTCTGCCAAGAACCCGCAGCCCGCCCAGGCATGGCTGTTCAGTGACCA
CCTCATCCAGCAGCAGGGGAAGTGCCTGGCTGCCACCTCCACCTTAATGTCCTCCCCTGGATC
CCCAGTCATACTGCAGATGTGCAACCCTAGAGAAGGCAAGCAGAAATGGAGGAGAAAAGGA
TCTTTCATCCAGCATTCAGTCAGTGGCCTCTGCCTGGAGACAAAGCCTGCCCAGCTGGTGACC
AGCAAGTGTCAGGCTGACGCCCAGGCCCAGCAGTGGCAGCTGTTGCCACACACATGA
T16 LECTIN Amino acid sequence
SVATRIEQRKKMNCKSFRWYLENVYPELTVPVKEALPGIIKQGVNCLESQGQNTAGDFLL
GMGICRGSAKNPQPAQAWLFSDHLIQQQGKCLAATSTLMSSPGSPVILQMCNPREGKQKWRRKGSF
IQHSVSGLCLETKPAQLVTSKCQADAQAQQWQLLPHT*

In this Example we have defined minimal sequences of functional lectin domains based on multiple sequence alignments. It is clear that changes in the length of sequences used may not affect functionality of the lectins. Such changes could constitute, for example, plus or minus 10-20 amino acid residues of the GalNAc-transferase sequence at their amino or carboxy termini. For example, the GalNAc-T1 lectin domain may comprise 10-20 fewer amino acid residues at its carboxy and/or amino termini than shown in Table III's T1 lectin domain sequence; i.e. the T1 lectin domain could, for example, stretch from amino acids 403-549 of the GALNT1 sequence, or, for example, from amino acids 413-539 of the GALNT1 sequence. Additionally, the GalNAc-T1 lectin domain may comprise 10-20 more amino acid residues at its carboxy and/or amino termini than shown in Table III's T1 lectin domain sequence; i.e. the T1 lectin domain could, for example, stretch from amino acids 383-569 of the GALNT1 sequence, or, for example, from amino acids 373-579 of the GALNT1 sequence.

Sf9 cells were co-transfected with pACGP67-GalNAc-transferase soluble expression constructs and Baculo-Gold™ DNA (Pharmingen) as previously described³³. Briefly, 0.4 μg DNA was mixed with 0.1 μg Baculo-Gold DNA and co-transfected in Sf9 cells in 24-well plates. Ninety-six hours post-transfection recombinant virus was amplified in 6-well plates at dilutions of 1:10 and 1:50. Titer of amplified virus was estimated by titration in 24-well plates. For large scale production and purification of recombinant secreted enzymes and lectins the amplified vira were used to infect High Five™ cells grown in serum free medium (Invitrogen) in upright roller bottles shaking at 140 rpm in 27° C. waterbaths. Recombinant proteins were purified by nickel NiTA chromatography using nickel agarose (Qiagen) as recommended by the manufacturer or by consecutive chromatographies on Amberlite, S-sepharose and Mono-S as previously described³⁹.

### 2. Direct Binding Assay for Determination of Carbohydrate Specificity of Polypeptide GalNAc-Tranferase Lectins Using Soluble GalNAc-Transferase Enzymes

GalNAc-transferase lectins were previously shown to direct GalNAc-glycopeptide substrate specificities of some GalNAc-transferase². The mechanism by which the lectin domains mediate this specificity is unknown but the finding that the monosaccharide GalNAc selectively inhibits GalNAc-glycopeptide specificity of some isoforms suggested that the putative lectin domains were involved in an interaction with the substrate at least partly through the GalNAc-residue. Nevertheless, it has not been possible in the past despite many different attempts to demonstrate direct binding of the enzyme protein or fragments hereof to glycopeptides or saccharides² (PCT WO 01/85215 A2). In this Example a binding assay using HIS-tagged affinity purified and biotinylated secreted enzyme was developed. MS-tagged secreted human GalNAc-T2 and -T4 were prepared from pAC-GP67-T2-sol and pAC-GP67-T4-sol cDNA^{10,32} by PCR as described in Example 1.

Secreted GalNAc-T2 and -T4 and variant proteins were obtained from infected High Five™ cells grown in serum-free medium (Invitrogen) in upright roller bottles shaken 140 rpm in waterbaths at 27° C. Purification of the recombinant proteins were performed by iminodiacetic acid metal affinity chromatography (IMAC) Ni²⁺-charged (QIAGEN). Elution was achieved with 250 mM imidazole in 50 mM sodium phosphate (pH 8.0) and 500 mM NaCl. In some cases, recombinant proteins were purified by consecutive ionexchange chromatographies as developed and described previously³⁹, before final purification by Ni²⁺-chromatography. Proteins eluted were dialyzed three times against PBS (10 mM sodium phosphate (pH 7.4) 150 mM NaCl) and concentrated by centrifugal filter device (Millipore; 10,000 kDa cut off). Purity was analyzed by SDS-PAGE under reducing conditions, and stained for proteins with Coomassie Blue R 250.

Protein biotinylation was made as previously reported⁴⁹. The pH of 1 ml purified protein (0.3 mg/ml) in PBS was adjusted to pH 9 with 1 M NaOH and 40 μl N-hydroxy-succinimidobiotin (Sigma) dissolved in DMF (10 mg/ml) was added. The solution was mixed end-over-end for 2 hours at room temperature, and dialyzed three times against PBS and an equal volume of glycerol was added. The biotinylated proteins were stored at −20° C. in 50% glycerol until use.

Glycosylation of MUC1 peptides (0.1 mM) was made in 20 mM cacodylate buffer (pH 8.0), 10 mM MnCl₂, 10 mM UDP-GalNAc, and 20 μg purified polypeptide GalNAc-T1 or -T2 with or without subsequent glycosylation with GalNAc-T4 at 37° C. during overnight. Glycopeptides were purified by C-18 reverse phase HPLC. Peptides were custom synthesized by Neosystems (Strasbourg). Biotinylated Helix Pomatia lectin (HPA) was from KemEnTec (Denmark). Anti-MUC1 HMFG2 monoclonal antibody was a generous gift from Joyce Taylor-Papadimitriou. Anti-MUC15E5 monoclonal antibody was developed by immunizing Balb/c mice with 60-mer MUC1 tandem repeat peptide glycosylated with 5 moles GalNAc per repeat. Monoclonal antibodies to the lectin domains of human GalNAc-T2 and -T4 were developed as previously described^{10,50}.

Direct binding ELISA assay was developed as follows: Polystyrene microtiter plates (Maxisorb, Nunc, Denmark) were coated with peptides or enzymatically glycosylated glycopeptides in PBS overnight at 4° C. Plates were washed and blocked with 0.1% Tween20 and 0.2% BSA in PBS for 1 h at room temperature, followed by incubation with biotinylated proteins in PBS with 0.05% Tween20 for 2 h at room temperature. After four washes with PBS, plates were incubated with 1:2,000 dilution streptavidin-HRP (Sigma) in PBS for 30 min at room temperature and washed four times with PBS. Development was performed with 0.5 mg/ml o-phenylenediamine and 0.02% H₂O₂ at room temperature for 15 min, and reaction stopped by adding 100 μl/well of 0.5 N H₂SO₄. Competitive inhibition assays were done at end-point titers of GalNAc-transferase proteins with one hour preincubations with inhibitor.

In initial binding experiments it was determined that secreted GalNAc-T2 and -T4 could bind their peptides substrates in the presence of 5 mM UDP and Mn⁺⁺ (may be substituted with other divalent cation), whereas no binding was observed in the absence or when 10 mM EDTA was included. This correlates with our previous findings that GalNAc-T2 can be purified on an acceptor substrate peptide in the presence of UDP and Mn⁺⁺, and can be eluted by removing UDP in EDTA³². This binding is mediated by the catalytic unit of the enzyme, which exposes the acceptor-binding pocket only in the presence of UDP and Mn⁺⁺, predicted by the ordered catalytic reaction.

In order to selectively evaluate the binding characteristics of the putative lectin domain, assays were carried out in the absence of UDP and Mn⁺⁺. Significant binding to GalNAc-peptides was found for both GalNAc-T2 and -T4 (FIG. 4).

We have previously shown that GalNAc-T4 with a single amino acid change in the lectin domain selectively impairs the GalNAc-glycopeptide substrate specificity of this enzyme². In agreement with this the lectin mutated enzyme protein did not bind GalNAc-glycopeptides. The binding to GalNAc-glycopeptides were unaffected by Ca++ and EDTA further confirming that the catalytic units of the enzyme proteins are not involve in binding.

GalNAc and GalNAcα-benzyl have previously been shown to inhibit the GalNAc-glycopeptide substrate specificity of GalNAc-T4² (PCT WO 01/85215 A2). Lectins and antibodies to carbohydrates usually recognize the anomeric configuration of the sugar structures they bind. However, surprisingly, both GalNAc-T2 and -T4 exhibit equal inhibition with GalNAcα-benzyl (Sigma) and GalNAcβ-benzyl (NuRx, Alberta Research Council (FIG. 5).^{58,59} Similar results were obtained with other aryl derivatives.

The methods described in this Example utilize recombinant polypeptide GalNAc-transferases in binding assays and excludes potential binding activity through the catalytic unit. It is clear that recombinant polypeptide GalNAc-transferases with mutations that inactivates the binding activity of the catalytic unit can be used, as well as any truncation of the enzyme protein that eliminate the binding activity of the catalytic unit.

While this binding assay establishes a method for screening for inhibitors of lectin mediated binding mediated through the lectins of human GalNAc-T2 and GalNAc-T4, it is clear that the same method with modifications can be applied to all animal and mammalian polypeptide GalNAc-transferases with a functional lectin domain. The ligand target used in this Example is a GalNAc-MUC1 glycopeptide produced enzymatically from synthetic peptides. It is clear that GalNAc-glycopeptides based on any number of peptides with GalNAc attached can be used as target for the binding assay. It is also clear that the assay developed can be modified to accommodate high through put screening by any assay method available in the prior art that can detect and quantify binding between the polypeptide GalNAc-tranferase mediated through its lectin domain and a suitable ligand.

### 3. Direct Binding Assay for Determination of Carbohydrate Specificity of Polypeptide GalNAc-Tranferase Lectins Using Truncated GalNAc-Transferase Lectin Domains

In Example 2 it was established that secreted soluble enzymatically active proteins of GalNAc-T2 and -T4 bind GalNAc-MUC1 glycopeptides, and that GalNAc could inhibit the binding. The catalytic unit of polypeptide GalNAc-transferase can interact and bind acceptor substrate peptides and possible glycopeptides³⁸, however, binding studies without donor substrates (UDP), and in the presence of EDTA to chelate Mn²⁺, suggested that the binding was not mediated through the catalytic unit. In this Example direct binding to GalNAc-glycopeptides through the lectin domains of polypeptide GalNAc-transferases GalNAc-T2 and -T4 was established. Attempts to express C-terminal truncated GalNAc-transferase proteins failed due to low secretion rate presumable related to folding problems and intracellular degradation. Similar phenomenon has recently been reported for GalNAc-T1¹⁴. Numerous attempts to express isolated lectin domains in insect cells and P. pastoris have failed due to low expression and apparent degradation. As described in Example 1 successful expression was finally achieved with constructs truncated as described in Table II using an expression vector with a N-terminal HIS tag and thrombin cleavage site as well as a T7 tag. HIS-tagged truncated GalNAc-T2 and -T4 lectins were expressed and purified as described in Example 2. Lectins were used in binding studies directly or after biotinylation as described in Example 2. In binding studies using lectins without biotinylation antibodies to the HIS-tag and the T7-tag, or in some experiments antibodies raised to GalNAc-T2 and -T4 enzymes were used to detect binding.

Inhibition experiments were used to further define the binding specificity of GalNAc-T2 and -T4 secreted soluble enzymes as well as lectin domains compared to Helix Pomatia lectin (Table IV). GalNAcα- and GalNAcβ-aryl structures inhibited binding of both enzymes and isolated lectins at comparable levels. In contrast, Helix Pomatial showed strong preference for GalNAcα-derivatives. Gal and other sugars had no inhibitory effect. Interestingly, UDP-GalNAc was not a significant inhibitor of the GalNAc-transferase lectin binding, but a strong inhibitor of Helix Pomatia binding.

TABLE IV
Inhibition of carbohydrate-binding proteins
by using saccharides and related structures.

| Carbohydrates | T2ld | sT2 | T4ld | sT4 | HPA |
|---|---|---|---|---|---|
| Glc | >100^{a} | >100 | >100 | >100 | >100 |
| GlcNAc | >100 | >100 | >100 | >100 | 8 |
| BzlαGlcNAc | >100 | >100 | >100 | >100 | 2 |
| BzlβGlcNAc | >100 | >100 | >100 | >100 | >20 |
| Gal | >100 | >100 | >100 | 50 | >100 |
| MeαGal | >100 | >100 | >100 | 50 | >100 |
| MeαGal | >100 | >100 | >100 | 40 | >100 |
| GalNAc | 37 | 15 | 5 | 1 | 2 |
| BzlαGalNAc | 20 | 15 | 5 | 1 | 0.5 |
| PhlαGalNAc | 15 | 5 | 5 | 1 | 1 |
| oNPαGalNAc | 12 | 7 | 5 | 0.5 | 1.5 |
| oNPβGalNAc | >12 | 10 | 8 | 1 | >20 |
| pNPαGalNAc | >10 | >10 | 10 | 1 | 2 |
| pNPβGalNAc | >10 | >10 | 8 | 0.8 | >20 |
| UDPαGalNAc | >100 | 65 | 50 | 30 | 2 |
| UDP | >100 | >100 | >100 | >100 | >100 |
| Lactose | >100 | >100 | >100 | >100 | ND |
| EDTA | >10 | >10 | >10 | >10 | >10 |

^{a}Concentration (mM) required for 50% inhibition (IC50).
ND, not determined.

While this binding assay establishes a method for screening for inhibitors of isolated human GalNAc-T2 and GalNAc-T4 lectins, it is clear that the same method with modifications can be applied to all animal and mammalian polypeptide GalNAc-transferase lectins. The ligand target used in this Example is a GalNAc-MUC1 glycopeptide produced enzymatically from synthetic peptides. It is clear that GalNAc-glycopeptides based on any number of peptides with GalNAc attached can be used as target for the binding assay. It is also clear that the assay developed can be modified to accommodate high through put screening by any assay method available in the prior art that can detect and quantify binding between the isolated lectin and a suitable ligand.

The methods described in this Example utilize recombinant GalNAc-transferase lectins in binding assays which excludes potential binding activity through the catalytic unit. It is clear that recombinant polypeptide GalNAc-transferases with mutations that inactivates the binding activity of the catalytic unit can be used, as well as any truncation of the enzyme protein that eliminate the binding activity of the catalytic unit.

### 4. Establishment of Cell Line Model Systems for Cell Surface Expression of Mucin and Secreted Mucin—Stably Transfected CHO and CHO 1d1D Cells

Cell lines and expression constructs: Wild type Chinese Hamster Ovary cells (CHO) and the glycosylation deficient mutant cell line CHO 1d1D⁴⁰ were stably transfected with a full coding MUC1 construct (MUC1F, supplied by M. A. Hollingsworth, Nebraska, USA) containing 32 tandem repeats using the pcDNA3 vector (Invitrogen). A secreted MUC1 construct (MUC1-IgGHIS) was generate by insertion of mouse IgGγ2a domain fused to 6× histidine tag at the BsU36I site downstream of the tandem repeat region of MUC1F⁵¹. Cells were generally grown in Hams F12 containing 10% Fetal Bovine Serum at 37° C. at 5% CO₂, and plated 12-24 hours prior to transfection in 6 well plates and grown to approximately 50% confluency. One hour before transfection cells were washed in serum free medium Optimem (Invitrogen) and cells were transfected with 1-2 μg DNA using the Lipofectamine plus reagent (Invitrogen) in a total volume of 1 mL as recommended by the supplier. Three hours after the transfection one mL of Hams F12 containing 10% Fetal Bovine Serum was added and cells grown 24-48 hours before medium was replaced with 2 mL Hams F12 containing 10% Fetal Bovine Serum. Two to three days after transfection cells were trypzinized and plated in 75 mL T-flasks or in 24/96 well microtiter plates in the same medium containing the appropriate selection agent (1 mg/ML G418 or 0.4 mg/mL Zeocin). Selection medium was changed twice weekly until clones appeared. The medium used for CHO 1d1D cells included 1 mM GalNAc and 0.1 mM Gal. Transfectant clones were selected by immunocytology with anti-MUC1 antibodies and SDS-PAGE western blot analysis to demonstrate cell surface expression and secretion of MUC1.

Immunocytology: Two different procedures were applied: i) For general screening purposes, cells grown in plates or flasks were trypsinized, washed in saline, and airdried on multiwell coverslides. Slides were fixed in ice-cold acetone and stained with monoclonal antibodies and FITC-conjugated rabbit anti-mouse Ig as previously described⁵⁰. ii) For analysis of cell surface expression, cells were seeded in 6 well plates and grown for 6 hours in Hams F12 medium with serum until approximately 30-50% subconfluent. Medium was hereafter replaced with Optimem supplemented with 1.0 mM GalNAc and 0.1 mM Gal and cells grown for 18 to 42 hours. Cells were washed once in PBS (phosphate buffered saline without Calcium and Magnesium) and subsequently fixed in 2 ml 3% paraformaldehyde at 25° C. for 20 min followed by three washes with PBS. Free aldehyde groups were quenched by incubating in 2 mL 50 mM Ammonium Chloride in PBS for 10 min, followed by three washes with PBS and three washes with 5 min incubations each with PBS containing 0.2% Fish Skin Gelatin (Sigma). Immunostaining of cells was performed by incubation with monoclonal antibodies for 40 min at 25° C., followed by three washes with PBS and three washes of 5 min each with PBS containing 0.2% Fish Skin Gelatin. Subsequently, cells were incubated with FITC-conjugated rabbit anti-mouse Ig (Dako, F261) diluted 1:150 in PBS containing 0.2% Fish Skin Gelatin) for 20 min at 25° C., followed by the same washing procedure, after which wells were cut out of plates and mounted with glycerol as for glass slides.

Characterization of wild type and MUC1 stable transfectant CHO cells: Several representative clones expressing the full coding or secreted MUC1 construct were selected and characterized for expression of MUC1 as well as O-glycosylation. Wild type CHO/MUC1F-clone1 expressed MUC1 at the cell surface as detected by anti-MUC1 monoclonal antibodies on non-permeabilized cells, while wild type CHO/MUC1sol-cloneC4 only was labeled weakly at the surface. Staining with a panel of anti-MUC1 antibodies of permeabilized cells showed intracellular accumulation of MUC1 detected by HMFG2 (general anti-MUC1 reactive,⁵²), SM3 (reactive with cancer-associated MUC1,⁵²), VU-4H5 (reactive with low density O-glycosylated MUC1,⁵³), VU-2G7 (reactive with high density O-glycosylated MUC1,⁵⁴), and a novel antibody 5E5 reactive exclusively with STn/Tn-glycosylated MUC1 glycoforms. In contrast, staining of non-permeabilized cells were only reactive with the anti-MUC1 antibodies HMFG2, SM3 and weakly VU-2G7. Analysis of O-glycosylation using a panel of anti-carbohydrate monoclonal antibodies revealed that wild type CHO cells label very weakly with anti-T antibodies (HH8, 3C9,⁵⁵ at the surface after neuraminidase treatment, while untreated cells are negative indicating that wild type CHO cells express very little O-glycoproteins and the glycosylation is mainly of sialylated core 1 structure (ST) (FIG. 6). Antibodies to Tn (1E3, 5F4,⁵⁵) were weakly reactive without and with neuramimidase treatment and antibodies to STn (TKH2, 3F1, 55) were negative. Staining with the lectins PNA (T), HPA (Tn), SNA (α2,6sialic acid) and MAA (α2,3sialic acid) were in agreement except the finding of weak reactivity with SNA indicating some presence of α2,6 linked sialic acids which may be derived from N-linked or O-linked glycans. These results demonstrate that the main form of O-glycosylation found on MUC1 expressed in wild type CHO is the sialyl-T structure as found for other recombinant glycoproteins⁵⁶.

Staining of permeabilized wild type CHO/MUC1sol-clone-C4 with anti-MUC1 antibodies revealed strong intracellular expression of MUC1 with HMFG2, SM3, vu-4H5, and vu-2G7 (FIG. 6). Staining with anti-carbohydrate antibodies revealed strong intracellular staining with anti-T after neuraminidase only, while anti-Tn only labeled weakly. These results indicate that the main glycosylation of MUC1 in CHO wild type is ST similar to untransfected cells.

In order to characterize the secreted MUC1 product SDS-PAGE western blot analysis of harvested culture medium of confluent cultures were performed. Ten to twenty μL culture supernatant was analysed directly or treated with 0.1 U/mL neuraminidase (C. Perfringes VI, Sigma) for 30-60 min at 37° C. Samples were mixed with SDS sample buffer, reduced with DTT, and run on precast 4-20% gels (Biorad). As shown in FIG. 7 anti-MUC1 antibodies detected two forms of MUC1 in the medium; a low molecular weight form migrating as 130-140 kd corresponding to unglycosylated product, and a high molecular weight form migrating above 250 kd. The high molecular weight form was sensitive to neuraminidase treatment as evidenced by a marked shift and retardation in migration. It is known that sialylated glycoproteins migrate aberrantly and often desialylation results in slower migration by SDS-PAGE analysis regardless of the mass. Interestingly, the antibody VU-4H5 reacted mainly with the unglycosylated form and only a very weak band was found in the high molecular weight forms after neuraminidase treatment. This result indicates that the PDTR region is O-glycosylated as the VU-4H5 antibody was previously found to tolerate O-glycosylation most positions in the tandem repeat except the PDTR region 53. In agreement with this the antibody VU-2G7 raised against a MUC1 GalNAc-glycopeptide with only one GalNAc per repeat attached in the PDTR region reacted strongly with the secreted MUC1. Furthermore, reactivity with the anti-T antibody after neuraminidase treatment showed that the main type of O-glycosylation on secreted MUC1 was sialylated-T. Anti-Tn and STn produced no staining.

Characterization of mutant and MUC1 stable transfectant CHO 1d1D cells: CHO 1d1D cells stably transfected with full coding MUC1, e.g. CHO1d1D/MUC1F-clone2, expressed MUC1 at the cell surface as detected by anti-MUC1 antibodies when cells were grown in GalNAc and Gal (FIG. 8). Cells were seeded at approximately 30-50% confluency (approx. 0.2×10⁶ per 6 well plate) in Hams F12 medium supplemented with 10% Fetal Bovine serum and grown for 6 hours. Medium was replaced with Optimem with or without 1.0 mM GalNAc and/or 0.1 mM Gal, and cells grown for 18-36 hours after which cells were trypsinised and washed in saline and processed as described for immunocytology. CHO1d1D/MUC1F-clone2 cells grown in the absence of sugars and analysed after permeabilization produced very little MUC1 detectable by HMFG2 but not by 5E5. In contrast, cells grown in the presence of only GalNAc strongly expressed MUC1 as evaluated by HMFG2 and 5E5, specifically reactive with GalNAc-MUC1. In agreement with reactivity with 5E5 these cells also labeled strongly with anti-Tn antibodies, 5F4 and 1E3, while anti-T antibodies, HH8 and 3C9, did not label the cells. Very weak or no staining with anti-STn antibodies (3F1 and TKH2) indicates that α2,6 sialylation to form STn does not occur in CHO 1d1D cells. CHO1d1D/MUC1F-clone2 cells grown in the presence of both GalNAc and Gal show reactivity at the surface with anti-T antibodies (HH8 and 3C9) only after neuraminidase pretreatment, confirming previous results that the predominant glycoform in CHO cells is sialyl-T (FIG. 8). No staining with anti-Tn or STn antibodies was detected with cells grown in both Gal and GalNAc. CHO1d1D/MUC1F-clone2 cells grown in the absence of GalNAc and Gal or in the presence of GalNAc alone showed no reactivity with anti-Tn and T antibodies or lectins (DBA, HPA, VVA, PNA, not shown) were detected indicating complete lack of O-glycosylation (FIG. 8). Cell surface expression of MUC1 was detected in CHO1d1D/MUC1F-clone2 cells grown in the presence of GalNAc, while cells grown without GalNAc showed no or only weak expression of MUC1 at the surface (FIG. 8). Surface expression of MUC1 was detected with HMFG2 in cells grown in GalNAc as well as cells grown in both Gal and GalNAc, however, expression analysed with the Tn/STn-MUC1 glycoform specific antibody 5E5 revealed surface expression only with cells grown in the presence of GalNAc (FIG. 8). This latter finding is in agreement with the O-glycosylation pattern determined above in these cells.

CHO1d1D/MUCsol-cloneD5 secretes MUC1 to the culture medium, and permeabilized cells immunostain with antibodies to MUC1 in the cytoplasm. Cells were grown in Hams F12 medium supplemented with 10% Fetal Bovine serum and seeded at a density of 0.2×10⁶ in 6 well plates. Following growth for 6 hours, the medium was replaced with Optimem supplemented with 1 mM GalNAc, 0.1 mM Gal, or 1 mM GalNAc and 0.1 mM Gal, and cells grown for 18-72 hours. Secretion of MUC1 was monitored by immunochemical assays of culture supernatants at differing time points. SDS-PAGE western blot analysis was performed with 5 μl culture supernatant mixed with 5 μl of 2×SDS sample buffer containing 1 mM DTT. Samples were heated to 100° C. for 2 min and loaded on a precast 4-20% gradient gel and run at 125 V for 75 min. Transfer to nitrocellulose membrane was performed by electroblotting using Biorad Mini Trans Blot apparatus at 350 mA for 1 hour. Membranes were blocked with 15% skimmed milk prepared in dH₂O for 2 hours and stained with anti-MUC1 and anti-carbohydrate monoclonal antibodies for 18 hours at 4° C., followed by washing with Tris buffered saline (TBS) (10 mM Tris pH 8.0 with 8.5% NaCl) 5 times for 5 min, and incubation with biotinylated rabbit anti-mouse IgG subclass specific antibodies (1:1000 dilution in TBS) for 1 hour at 25° C. Following 5 washes for 5 min each in TBS, membranes were incubated in HRP conjugated Streptavidin (1:3000 dilution in TBS) for 30 min at 25° C. After 5 washings of 5 min each in TBS the blot was developed in 0.04% 4-Chloro-1-Naphthol prepared in 50 mM Tris-HCl (pH 7.4) containing 0.025% H₂O₂. Similar to the findings with full coding MUC1 expressed at the cell surface of CHO 1d1D cells, glycosylation of the secreted MUC1 was dependent on Gal and GalNAc sugars in culture medium. Cells grown without sugars produced and secreted low amounts of a low molecular weight MUC1 molecule of apparent mw of 120-130 kd without glycosylation detectable by HMFG2 but not 5E5 or anti-Tn and anti-T antibodies (FIG. 9). In contrast, cells grown in 1 mM GalNAc secreted MUC1 glycosylated with GalNAc (Tn) as evidenced by reactivity with both HMFG2 and 5E5 as well as anti-Tn antibodies (FIG. 9). The apparent molecular weight of secreted Tn-MUC1 was 250-300 kd and no significant shift in migration was observed with pretreatment with neuraminidase (0.1 U/ml for 30 min at 37° C.), suggesting lack of α2,6sialylation (STn). This was confirmed by lack of staining with anti-STn antibodies. Cells grown in both 0.1 mM Gal and 1 mM GalNAc produced and secreted MUC1 with sialylated core 1 (T) glycoforms reactive with HMFG2 but not 5E5 (FIG. 9). Pretreatment with neuraminidase resulted in a significant shift in migration and reactivity with anti-T antibodies as well as the lectin PNA. Two novel anti-MUC1 antibodies described recently have been suggested to react with the MUC1 tandem repeat sequence without (Nab VU-4H5) or with (Mab VU-2G7) O-glycans attached in the central immunodominant epitope -PDTR-. Analysis of secreted MUC1 produced in CHO 1d1D cells grown without GalNAc show reactivity with unglycosylated MUC1 migrating at mw 120-130 kd with VU-4H5, while no or only weak reactivity was observed when grown in GalNAc with or without Gal (FIG. 10). In contrast, the Mab VU-2G7 reacted strongly with MUC1 migrating at 250-300 kd secreted from cells grown in GalNAc with or without Gal (FIG. 10). Although, Mab VU-2G7 reacted weakly with unglycosylated MUC1 the combined results suggest that MUC1 produced in CHO 1d1D cells carry O-glycans on all five sites of the tandem repeat.

### 5. The Inhibitor GalNAcα-benzyl Inhibits MUC1 Expression Independently of O-glycan Processing

As shown in Example 4 CHO1d1D/MUC1F-clone2 cells grown in the presence of GalNAc but not Gal have limited O-glycosylation capacity, only produce the Tn glycoform of MUC1, but expresses comparable levels of MUC1 at the cell surface as in wild type CHO cells or in CHO 1d1D cells grown in both GalNAc and Gal. This suggested that cell surface expression was not related to O-glycosylation and particular glycoforms as previously proposed (for a review see Huet). We therefore investigated the effect of treatment with GalNAcα-benzyl of CHO1d1D/MUC1F-clone2 cells grown in the presence of GalNAc. CHO1d1D/MUC1F-clone2 cells were seeded in 6 well plates at a density of 0.2×10⁶ per well and were grown for 6 hours in Hams F12 medium with serum until approximately 30% subconfluent. Medium was hereafter replaced with Optimem supplemented with 1.0 mM GalNAc or 1.0 mM GalNAc and 0.1 mM Gal with or without the inhibitors GalNAcα-benzyl, GalNAcβ-benzyl or the control GlcNAcα-benzyl. After 18 hours, the medium was replaced with fresh Optimem containing the sugars and benzyl derivatives as above and grown for 12-48 hours. Initially we analyzed surface expression of MUC1 by immunocytology. Cells were washed once in PBS-CMF (phosphate buffered saline Calcium and Magnesium free) after carefully removing the medium from the wells and subsequently fixed in 2 ml 3% paraformaldehyde at 25° C. for 20 min followed by three washes with PBS-CMF 3 times. Free aldehyde groups were quenched by incubating in 2 mL 50 mM Ammonium Chloride in PBS-CMF for 10 min, followed by three washes with PBS-CMF and three washes with 5 min incubations each with PBS-CMF containing 0.2% Fish Skin Gelatin (Sigma). Immunostaining of cells was performed by incubation with monoclonal antibodies for 40 min at 25° C., followed by three washes with PBS-CMF and three washes of 5 min each with PBS-CMF containing 0.2% Fish Skin Gelatin. Subsequently, cells were incubated with FITC-conjugated rabbit anti-mouse Ig (Dako F261) diluted 1:150 in PBS-CMF containing 0.2% Fish Skin Gelatin) for 20 min at 25° C., followed by the same washing procedure, after which wells were cut out of plates and mounted with glycerol as for glass slides. FIG. 11 shows that treatment with 1 mM GalNAcα-benzyl, produced strong inhibition of cell surface expression of MUC1, while treatment with a similar control benzyl derivative showed no inhibition. GlcNAcα-benzyl was chosen as a control because this sugar does not serve as a substrate for mammalian glycosyltransferases and hence was not expected to interfere with O-glycosylation in CHO cells. Most anti-MUC1 antibodies reacted with cells grown in GalNAc including VU-2G7 and 5E5, and only VU-4H5 did not react. Reactivity with VU-2G7 indicates that the -PDTR- region is O-glycosylated, while reactivity with 5E5 confirms that the glycoforms of surface MUC1 is mainly or exclusively Tn.

We next analysed the expression of MUC1 produced by CHO1d1D/MUC1F-clone2 cells by SDS-PAGE western analysis. Cells were grown for 24 hours or 48 hours in the presence of 1 mM GalNAc or 1 mM GalNAc and 0.1 mM Gal to limit core O-glycosylation to GalNAcα1-O-Ser/Thr and Galβ1-3GalNAcα1-O-Ser/Thr, respectively. Cells were further treated with 2 mM GalNAcα-benzyl, GlcNAcα-benzyl or no inhibitor. Cells were washed and lysed at 24 or 48 hours and the lysates subjected to immunoprecipitation with monoclonal antibody HMFG2, which broadly recognize MUC1 glycoforms. Immunoprecipitates were analysed by SDS-PAGE and western blot using HMFG2 antibody to detect MUC1 expression. As shown in FIG. 12 the MUC1 glycoforms at 24 hours expressed by cells grown in GalNAc or Gal and GalNAc migrated similarly with only little high molecular weight forms in both, indicating that synthesis of sialylated core 1 O-glycans were time limited. At 48 hours, MUC1 glycoforms migrating as higher molecular weight species were expressed more pronounced and selectively by cells grown in Gal and GalNAc. Treatment with GlcNAcα-benzyl produced the same glycoforms at similar intensity as cells without treatment. In striking contrast, treatment with GalNAcα-benzyl had significant effect after 48 hours. A significant reduction in MUC1 expression was found in cells grown in GalNAc as well as in Gal and GalNAc. In the latter case a significant shift in migration into two bands further confirmed that GalNAcα-benzyl also serves as an inhibitor of O-glycan extension and reduces O-glycosylation to GalNAcα1-O-Ser/Thr. Analysis of the same blots with the anti-MUC1 antibody 5E5 (FIG. 13) produced essentially the same results except that the antibody only labeled the lower migrating band of the two bands labeled by HNWG2 in cells grown in Gal and GalNAc and treated with GalNAcα-benzyl. This indicates some heterogeneity in glycosylation.

These results show for the first time that the effect of GalNAcα-benzyl on mucin transport and surface expression is independent of its effects on O-glycosylation in striking contrast to the prevailing hypothesis²⁶. Because cells grown in the presence of GalNAc do not produce core 1 (Galβ1-3GalNAcα1-O-Ser/Thr) O-glycosylation, GalNAcα-benzyl cannot serve as a competitive substrate for the core 1 β3galactosyltransferase and subsequently for sialyltransferases. GalNAcα-benzyl must therefore exert its function on mucin transport by another unknown mechanism.

The in vivo cell line model system developed is one example of a method to screen for inhibitors effects of one or more compounds on transport of mucins and O-linked glycoproteins in cells. The Example utilizes MUC1 but any mucin or O-linked glycoprotein could be used with appropriate expression constructs, antibodies and reagents. The developed cell model and modifications hereof can be used for high throughput screens of inhibitors in combination with or as a second screen after the binding assays disclosed in Examples 2 and 3.

### 6. Identification of a Novel Selective Inhibitor, GalNAcβ-benzyl, of Polypeptide GalNAc-Transferase Lectins that Inhibits MUC1 Expression without Affecting O-glycosylation

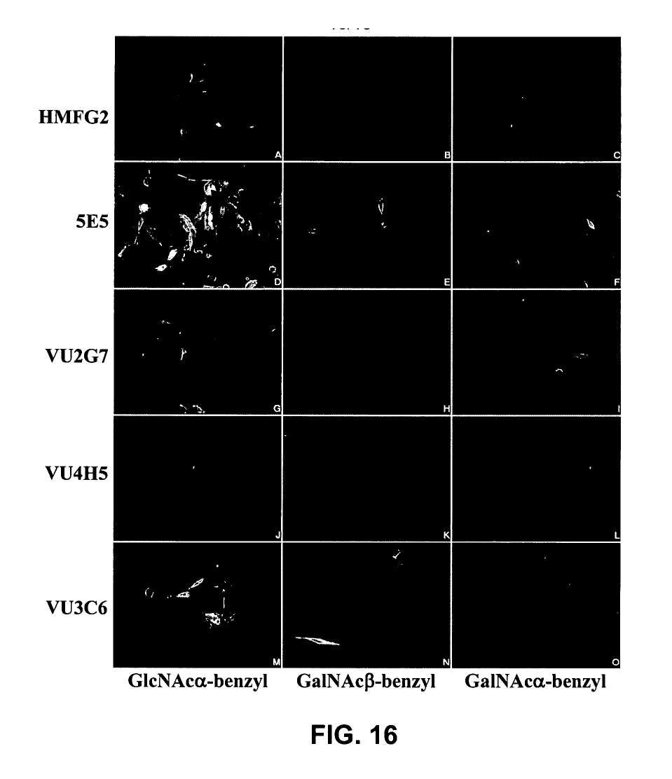

As shown in Examples 2 and 3, polypeptide GalNAc-transferases contain lectin domains with binding properties for GalNAc-peptides including GalNAc-MUC1 peptides. Since GalNAcα-benzyl was found to inhibit the binding properties of GalNAc-transferase lectins, we tested the possibility that the independent effect on mucin expression this O-glycosylation inhibitor has, could be related to an inhibitory effect on GalNAc-transferase lectins. In Examples 2 and 3 we found surprisingly that the lectin domains of several GalNAc-transferases in addition to GalNAcα-benzyl, which mimics the GalNAc-glycopeptide targets of the lectins, also were inhibited by βGalNAc derivatives. Initial tests with commercially available GalNAcβ and GalNAcα derivatives, p-nitrophenyl and umbrelliferyl did not produce significant effects in our model system. GalNAcβ-benzyl, the β-anomeric configuration of GalNAc-benzyl (there is a β linkage between the N-acetylgalactosamine and the benzyl ring), was custom synthesized by Alberta Research Council (Canada), and its structure was confirmed by mass spectrometry and ¹H-NMR. CHO1d1D/MUC1F-clone2 cells were grown for 12 hours in the presence of 1 mM GalNAc or 1 mM GalNAc and 0.1 mM Gal to limit core O-glycosylation to GalNAcα1-O-Ser/Thr and Galβ1-3GalNAcα1-O-Ser/Thr, respectively. Cells were then treated with 2 mM GalNAcα-benzyl, GalNAcβ-benzyl, or GlcNAcα-benzyl as control (GlcNAcα-benzyl was shown in Example 4 to have no effect). Cells were washed and lysed as described above after 36 hours and the lysates subjected to immunoprecipitation with anti-MUC1 monoclonal antibodies HMFG2 or 5E5. FIG. 14 illustrates that treatment with GalNAcβ-benzyl produced the same or better reduction in MUC1 expression as treatment with GalNAcα-benzyl in cells grown in GalNAc as well as in Gal and GalNAc. In cells grown in Gal and GalNAc MUC1 expression was reduced with GalNAcβ-benzyl treatment, but in contrast to cells treated with GalNAcα-benzyl, GalNAcβ-benzyl produced no change in the migration of MUC1 demonstrating that this inhibitor does not affect the O-glycan processing. The lack of immunoprecipitation of MUC1 by antibody 5E5 in cells grown in Gal and GalNAc indicates that MUC1, is glycosylated with more complex structures than GalNAcα1-O-Ser/Thr as recognized by this antibody. As shown in FIG. 16 the main O-glycan phenotype of CHO 1d1D cells grown in Gal and GalNAc is sialylated-T, and 5E5 does not react with MUC1, with T or sialylated T glycoforms of MUC1. FIG. 15 illustrates the same experiment as in FIG. 14 except that the detection antibody is 5E5 and only Tn and STn MUC1 glycoforms are visualised. This experiment confirms the strong inhibition of MUC1 expression in GalNAcα-benzyl and GalNAcβ-benzyl treated cells.

The finding that GalNAcα-benzyl and GalNAcβ-benzyl exhibit the same inhibitory effect on GalNAc-transferase lectin binding, and that they have similar effects on inhibition of MUC1 expression, clearly indicate that the effects these compounds have on mucin expression and secretion are directed through interaction with the lectin domains of polypeptide GalNAc-transferases. The effect GalNAcα-benzyl has on O-glycan processing is a separate phenomenon directed by its ability to serve as a competitive substrate for the core 1 β1,3galactosyltransferase.

GalNAcβ-benzyl is the first identified selective inhibitor of polypeptide GalNAc-transferase lectins and their roles in transport and secretion, which does not modulate O-glycosylation in cells (i.e. does not serve a substrate for mainly core 1 β3galactosyltransferase activities, α2,6sialyltransferase activities, and core 3 β3GlcNAc-transferase activities). Inhibitors structurally related to GalNAcβ-benzyl with the same properties, as identifiable by the binding assays disclosed in Examples 2 and 3, may be designed and synthesized to obtain higher affinity binders. Such inhibitors may be based on carbohydrates such as the monosaccharide GalNAc or modifications thereof, inhibitors may be based on structural and functional-mimetics such as polypeptides, glycopeptides, DNA, RNA, antibodies, and antibody fragments including phage antibodies, and inhibitors may be natural or synthetic organic or inorganic compounds. One common feature for such preferred inhibitors is the ability to inhibit the binding of one or more polypeptide GalNAc-transferase lectins to its binding ligand, such as GalNAc-glycopeptides and mucins as exemplified in Examples 2 and 3. Another feature of the novel inhibitor GalNAcβ-benzyl is its ability to enter living cells and reach the Golgi apparatus for in vivo binding to polypeptide GalNAc-transferase. The hydrophobic benzyl aglycone is one example of an aryl compound suitable for the β-anomeric configuration of GalNAc-R, but other aryl substituents include, without limitation, p-nitrophenyl, umbrelliferyl, and naphtalenmethanol. Any pharmaceutical carrier known in the art may be used to achieve the same effect. The appropriate carrier will be evident to those skilled in the art and will depend in large part upon the route of administration.

### 7. Inhibition of Secretion of Mucins

Because GalNAcα-benzyl exerts separate effects on O-glycan processing and mucin expression, the use of the novel selective inhibitor of mucin expression, GalNAcβ-benzyl, allow analysis of mucin expression and secretion in different cell line models. Examples of human cell lines (available from ATCC, USA) expressing and secreting mucins are without limitations LS174T, HT29, Colo205, CALU, MCF7, T47D, NCI-H292, and A549. Most human adenocarcinoma cell lines express and secret mucins and analysis with antibodies to detect protein expression or probes to detect mRNA can reveal the types and quantities of mucins. The human colon carcinoma cell line LS174T was previously shown to exhibit reduced secretion of mucin following treatment with GalNAcα-benzyl⁵⁷. This and other cell lines can be used to treat with 2 mM GalNAcβ-benzyl. In this Example we used wild type CHO/MUC1sol-cloneC4 and western blot analysis of medium of cells treated with 1-2 mM GalNAcα-benzyl, GalNAcβ-benzyl, or the control GlcNAcα-benzyl. Treatment with both GalNAcα-benzyl and GalNAcβ-benzyl showed inhibition of secreted MUC1 compared to control treated or non-treated cells. The inhibitory effect on mucin secretion can be quantified by a number of assays known to the skilled in the art including western blot, ELISA, gelfiltration, immunocapture, and other assays.

The in vivo cell line model system developed is one example of a method to screen for inhibitors effects of one or more compounds on secretion of mucins and O-linked glycoproteins in cells. The Example utilizes MUC1 but any mucin or O-linked glycoprotein could be used with appropriate expression constructs, antibodies and reagents. The developed cell model and modifications hereof can be used for high throughput screens of inhibitors in combination with or as a second screen after the binding assays disclosed in Examples 2 and 3.

### 8. GalNAcβ-benzyl Inhibits Mucin Glycosylation and MUC5AC Production in HT29MTX Cells

In order to evaluate GalNAcβ-benzyl as an inhibitor of mucin glycosylation and secretion we used the cultured colon carcinoma cells of mucin secreting phenotypes: HT-29 metotrexate (MTX) cells selected from the HT-29 cell line by culture in the presence of MTX (kind gift from G. Huet, France). The HT-29 MTX cells were selected to yield a goblet cell like phenotype with constitutive production of MUC5AC. In order to ensure proper goblet cell differentiation the HT29MTX cell line were continuously grown up to 21 days in the presence of 5 mM GalNAcα-benzyl, 5 mM GalNAcβ-benzyl, 5 mM GlcNAcα-benzyl and without inhibitor using culture conditions as described in Hennebicq-Reig et al. (Permanent exposure of mucin-secreting HT-29 cells to benzyl-N-acetyl-alpha-D-galactosaminide induces abnormal O-glycosylation of mucins and inhibits constitutive and stimulated MUC5AC secretion. Biochem J. 1998; 334:283-95). At 7, 14 and 21 days of continuous culture, media was collected and cells were fixed in 3% paraformaldehyde and stained for the presence of MUC5AC and specific carbohydrate structures using immuno-histochemistry with the following monoclonal antibodies: CLH2 recognizing MUC5AC (Reis C. et al. Immunohistochemical study of MUC5AC expression in human gastric carcinomas using a novel monoclonal antibody. Int. J. Cancer. 1997; 74:112-21), 1E3 recognizing Tn (GalNAcα linked to serine or threonine), 3C9 recognizing T (Galβ1-3GalNAcα linked to serine or threonine, and 3F1 recognizing Sialyl-Tn (NeuAcα2-6GalNAcα linked to serine or threonine. In order to evaluate the amount of MUC5AC secreted by HT-29 MTX cells with and without GalNAcβ-benzyl treatment, media were subjected to SDS-PAGE (Tris-NuPAGE Gels 14-21%) followed by Western blotting using polyclonal rabbit anti human MUC5AC antibody LUM5-1 (Carlstedt et al. MUC5AC, but not MUC2, is a prominent mucin in respiratory secretions. Glycoconj J. 1996; 13:839-47). Furthermore the viscosity of the media from cells with and without inhibitor was evaluated using viscosity micro-measurement.

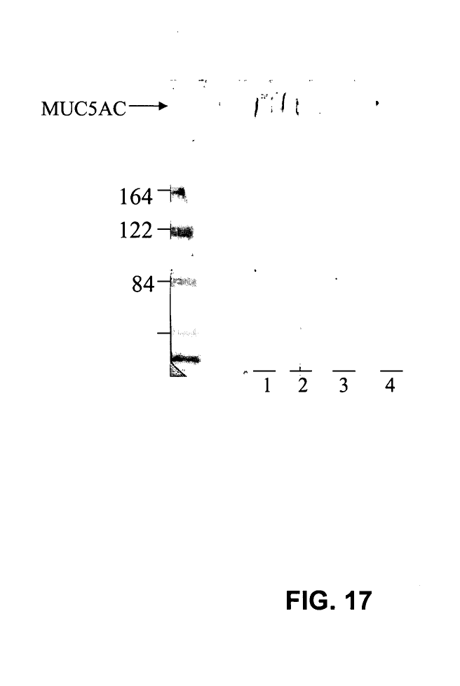

Treatment of cells with GalNAcβ-benzyl and GalNAcα-benzyl diminished secretion of MUC5AC as evaluated by western blot compared with GlcNAcβ-benzyl and cells treated with media alone (FIG. 17). Furthermore in accordance with earlier finding (Hennebicq-Reig et al. Permanent exposure of mucin-secreting HT-29 cells to benzyl-N-acetyl-alpha-D-galactosaminide induces abnormal O-glycosylation of mucins and inhibits constitutive and stimulated MUC5AC secretion. Biochem J. 1998; 334:283-95) treatment of cells with GalNAcα-benzyl (FIG. 18, Panel A) induced a storage phenotype with increased intracellular staining of MUC5AC. In contrast, cells treated with GalNAcβ-benzyl (FIG. 18, Panel B) did not lead to any storage disease but diminished intracellular staining of MUC5AC compared with and GlcNAcβ-benzyl (FIG. 18, panel C) and cells treated with media alone (FIG. 18, panel D). In addition the O-linked mucin carbohydrate structures T, Tn and sialyl-Tn were upregulated in cells treated with GlcNAcβ-benzyl compared to controls. Media from GalNAcβ-benzyl treated cells were less viscous compared with control media (data not shown). In conclusion the novel inhibitor GalNAcβ-benzyl inhibits both O-linked mucin glycosylation and MUC5AC secretion.

### REFERENCE LIST

1. Paulson, J. C. and Colley, K. J. Glycosyltransferases. Structure, localization, and control of cell type-specific glycosylation. J. Biol. Chem., 264: 17615-17618, 1989.
2. Hassan, H., Reis, C. A., Bennett, E. P., Mirgorodskaya, E., Roepstorff, P., Hollingsworth, M. A., Burchell, J., Taylor-Papadimitriou, J., and Clausen, H. The lectin domain of UDP-N-acetyl-D-galactosamine:polypeptide N-acetylgalactosaminyltransferase-T4 directs its glycopeptide specificities. J Biol Chem, 275: 38197-38205, 2000.
3. Schwientek, T., Bennett, E. P., Flores, C., Thacker, J., Hollmann, M., Reis, C. A., Behrens, J., Mandel, U., Keck, B., Schafer, M. A., Haselmann, K., Zubarev, R., Roepstorff, P., Burchell, J. M., Taylor-Papadimitriou, J., Hollingsworth, M. A., and Clausen, H. Functional conservation of subfamilies of putative UDP-N-acetylgalactosamine:polypeptide N-acetylgalactosaminyltransferases in Drosophila, Caenorhabditis elegans, and mammals. One subfamily composed of 1(2)35Aa is essential in Drosophila. J. Biol. Chem., 277: 22623-22638, 2002.
4. Hagen, F. K., Hazes, B., Raffo, R., deSa, D., and Tabak, L. A. Structure-Function Analysis of the UDP-N-acetyl-D-galactosamine:Polypeptide N-acetylgalactosaminyltransferase. Essential residues lie in a predicted active site cleft resembling a lactose repressor fold. J Biol Chem, 274: 6797-6803, 1999.
5. Hazes, B. The (QxW)3 domain: a flexible lectin scaffold. Protein Science, 5: 1490-1501, 1996.
6. Imberty A., Piller V., Piller F., and Breton C. Fold recognition and molecular modeling of a lectin-like domain in UDP-GalNac:polypeptide N-acetylgalactosaminyltransferases. Protein Eng., 10: 1353-1356, 1997.
7. BRETON, C. and Imberty, A. Structure/function studies of glycosyltransferases. Curr. Opin. Struct. Biol, 9: 563-571, 1999.
8. Bennett, E. P., Hassan, H., Hollingsworth, M. A., and Clausen, H. A novel human UDP-N-acetyl-D-Galactosamine:polypeptide N-acetylgalactosaminyltransferase, GalNAc-T7, with specificity for partial GalNAc-glycosylated acceptor substrates. FEBS Letters, 460: 226-230, 1999.
9. Ten Hagen, K. G., Bedi, G. S., Tetaert, D., Kingsley, P. D., Hagen, F., BALYS, M. M., BERES, T. M., Degand, P., and Tabak, L. A. Cloning and Characterization of a Ninth Member of the UDP-GalNAc:Polypeptide N-Acetylgalactosaminyltransferase Family, ppGaNTase-T9. J Biol Chem, 276: 17395-17404, 2001.
10. Bennett, E. P., Hassan, H., Mandel, U., Mirgorodskaya, E., Roepstorff, P., Burchell, J., Taylor-Papadamitriou, J., Hollingsworth, M. A., Merkx, G., Geurts van Kessel, A., Eiberg, H., Steffensen, R., and Clausen, H. Cloning of a human UDP-N-acetyl-α-D-galactosamine: polypeptide N-acetylgalactosaminyltransferase that complements other GalNAc-transferases in complete O-glycosylation of the MUC1 tandem repeat. J. Biol. Chem., 273: 30472-30481, 1998.
11. Ten Hagen, K. G., Tetaert, D., Hagen, F. K., Richet, C., BERES, T. M., Gagnon, J., BALYS, M. M., VanWuyckhuyse, B., Bedi, G. S., Degand, P., and Tabak, L. A. Characterization of a UDP-GalNAc:polypeptide N-acetylgalactosaminyltransferase that displays glycopeptide N-acetylgalactosaminyltransferase activity. J. Biol. Chem., 274: 27867-27874, 1999.
12. Muller, S., Alving, K., Peter-Katalinic, J., Zachara, N., Gooley, A. A., and Hanisch, F. G. High density O-glycosylation on tandem repeat peptide from secretory MUC1 of T47D breast cancer cells. J. Biol. Chem., 274: 18165-18172, 1999.
13. Muller, S., Goletz, S., Packer, N., Gooley, A. A., Lawson, A. M., and Hanisch, F. G. Localization of O-glycosylation sites on glycopeptide fragments from lactation-associated MUC1. J. Biol. Chem., 272: 24780-24793, 1997.
14. Tenno, M., Saeki, A., Kezdy, F. J., Elhammer, A. P., and Kurosaka, A. The lectin domain of UDP-GalNAc:Polypeptide N-acetylgalactosaminyltransferase 1 (GalNAc-T1) is involved in O-glycosylation of a polypeptide with multiple acceptor sites. J. Biol. Chem., 2002.
15. Jentoft, N. Why are proteins O-glycosylated? Trends. Biochem Sci 1990. Aug., 15: 291-294.
16. Tabak, L. A. In defense of the oral cavity: structure, biosynthesis, and function of salivary mucins. Annual Review of Physiology, 57: 547-564, 1995.
17. Van den Steen, P., Rudd, P. M., Dwek, R. A., and Opdenakker, G. Concepts and principles of O-linked glycosylation. Crit. Rev. Biochem Mol. Biol. 1998., 33: 151-208.
18. Taylor-Papadimitriou, J. and Epenetos, A. A. Exploiting altered glycosylation patterns in cancer: progress and challenges in diagnosis and therapy. Trends In Biotechnology, 12: 227-233, 1994.
19. Taylor-Papadimitriou, J. and Finn, 0. J. Biology, biochemistry and immunology of carcinoma-associated mucins. Immunology Today, 18: 105-107, 1997.
20. Scharfman, A., Lamblin, G., and Roussel, P. Interactions between human respiratory mucins and pathogens. Biochemical Society Transactions, 23: 836-839, 1995.
21. Rose, M. C. Mucins: structure, function, and role in pulmonary diseases. Am. J. Physiol. 1992. October, 263: L413-L429.
22. Thomsson, K. A., Carlstedt, I., Karlsson, N. G., Karlsson, H., and Hansson, G. C. Different O-glycosylation of respiratory mucin glycopeptides from a patient with cystic fibrosis. Glycoconj. J 1998. Aug., 15: 823-833.
23. Kuan, S. F., Byrd, J. C., Basbaum, C., and Kim, Y. S. Inhibition of mucin glycosylation by aryl-N-acetyl-alpha-galactosaminides in human colon cancer cells. J. Biol. Chem., 264: 19271-19277, 1989.
24. Huet, G., Hennebicq-Reig, S., de Bolos, C., Ulloa, F., Lesuffleur, T., Barbat, A., Carriere, V., Kim, I., Real, F. X., Delannoy, P., and Zweibaurn, A. GalNAc-alpha-O-benzyl inhibits NeuAcalpha2-3 glycosylation and blocks the intracellular transport of apical glycoproteins and mucus in differentiated HT-29 cells. J. Cell. Biol., 141: 1311-1322, 1998.
25. Byrd, J. C., Dahiya, R., Huang, J., and Kim, Y. S. Inhibition of mucin synthesis by benzyl-alpha-GalNAc in KATO III gastric cancer and Caco-2 colon cancer cells. Eur J Cancer 1995, 31A: 1498-1505.
26. Gouyer, V., Leteurtre, E., Zanetta, J. P., Lesuffleur, T., Delannoy, P., and Huet, G. Inhibition of the glycosylation and alteration in the intracellular trafficking of mucins and other glycoproteins by GalNAcalpha-O-bn in mucosal cell lines: an effect mediated through the intracellular synthesis of complex GalNAcalpha-O-bn oligosaccharides. Front Biosci., 6: D1235-D1244, 2001.
27. Alfalah, M., Jacob, R., Preuss, U., Zimmer, K. P., Naim, H., and Naim, H. Y. O-linked glycans mediate apical sorting of human intestinal sucrase-isomaltase through association with lipid rafts. Curr. Biol, 9: 593-596, 1999.
28. Yeaman, C., Le Gall, A. H., Baldwin, A. N., Monlauzeur, L., Le Bivic, A., and Rodriguez-Boulan, E. The O-glycosylated stalk domain is required for apical sorting of neurotrophin receptors in polarized MDCK cells. J Cell Biol 1997. Nov. 17, 139: 929-940.
29. Ulloa, F., Franci, C., and Real, F. X. GalNAc-a-O-Benzyl inhibits sialylation of de novo synthesized apical, but not basolateral, sialoglycoproteins and blocks lysosomal enzyme processing in a post-TGN compartment. J Biol Chem 2000. Apr. 5.
30. Muller, S, and Hanisch, F. G. Recombinant MUC1 probe authentically reflects cell-specific O-glycosylation profiles of endogenous breast cancer mucin: High-density and prevalent core2-based glycosylation. J. Biol. Chem., 2002.
31. Hassan, H., Bennett, E. P., Mandel, U., Hollingsworth, M. A., and Clausen, H. Control of Mucin-Type O-Glycosylation: O-Glycan Occupancy is Directed by Substrate Specificities of Polypeptide GalNAc-Transferases. In B. Ernst, G. W. Hart, and P. Sinaÿ (eds.), Carbohydrates in Chemistry and Biology, vol. 3, 1 ed, pp. 273-292. New York: Wiley-VCH, 2000.
32. White, T., Bennett, E. P., Takio, K., Sorensen, T., Bonding, N., and Clausen, H. Purification and cDNA cloning of a human UDP-N-acetyl-alpha-D-galactosamine:polypeptide N-acetylgalactosaminyltransferase. J. Biol. Chem., 270: 24156-24165, 1995.
33. Bennett, E. P., Hassan, H., and Clausen, H. cDNA cloning and expression of a novel human UDP-N-acetyl-alpha-D-galactosamine. Polypeptide N-acetylgalactosaminyltransferase, GalNAc-T3. J. Biol. Chem, 271: 17006-17012, 1996.
34. Bennett, E. P., Hassan, H., Mandel, U., Hollingsworth, M. A., Akisawa, N., Ikematsu, Y., Merkx, G., Geurts van Kessel, A., Olofsson, S., and Clausen, H. Cloning and Characterization of a Close Homologue of Human UDP-N-acetyl-α-D-galactosamine: Polypeptide N-Acetylgalactosaminyltransferase T3, designated GalNAc-T6: Evidence for Genetic but not Functional Redundancy. J. Biol. Chem., 274: 25362-25370, 1999.
35. White, K. E., Lorenz, B., Evans, W. E., Meitinger, T., Strom, T. M., and Econs, M. J. Molecular cloning of a novel human UDP-GalNAc:polypeptide N-acetylgalactosaminyltransferase, GalNAc-T8, and analysis as a candidate autosomal dominant hypophosphatemic rickets (ADHR) gene. Gene, 246: 347-356, 2000.
36. Toba, S., Tenno, M., Konishi, M., Mikami, T., Itoh, N., and Kurosaka, A. Brain-specific expression of a novel human UDP-GalNAc:polypeptide N-acetylgalactosaminyltransferase (GalNAc-T9). Biochim. Biophys. Acta, 1493: 264-268, 2000.
37. Homa, F. L., Hollander, T., Lehman, D. J., Thomsen, D. R., and Elhammer, A. P. Isolation and expression of a cDNA clone encoding a bovine UDP-GalNAc:polypeptide N-acetylgalactosaminyltransferase. J. Biol. Chem., 268: 12609-12616, 1993.
38. Sorensen, T., White, T., Wandall, H. H., Kristensen, A. K., Roepstorff, P., and Clausen, H. UDP-N-acetyl-alpha-D-galactosamine:polypeptide N-acetylgalactosaminyltransferase. Identification and separation of two distinct transferase activities. J. Biol. Chem., 270: 24166-24173, 1995.
39. Wandall, H. H., Hassan, H., Mirgorodskaya, E., Kristensen, A. K., Roepstorff, P., Bennett, E. P., Nielsen, P. A., Hollingsworth, M. A., Burchell, J., Taylor-Papadimitriou, J., and Clausen, H. Substrate specificities of three members of the human UDP-N-acetyl-alpha-D-galactosamine:Polypeptide N-acetylgalactosaminyltransferase family, GalNAc-T1, -T2, and -T3. J. Biol. Chem., 272: 23503-23514, 1997.
40. Kingsley, D. M., Kozarsky, K. F., Segal, M., and Krieger, M. Three types of low density lipoprotein receptor-deficient mutant have pleiotropic defects in the synthesis of N-linked, O-linked, and lipid-linked carbohydrate chains. J. Cell Biol., 102: 1576-1585, 1986.
41. Altschuler, Y., Kinlough, C. L., Poland, P. A., Bruns, J. B., Apodaca, G., Weisz, 0. A., and Hughey, R. P. Clathrin-mediated endocytosis of MUC1 is modulated by its glycosylation state. Mol. Biol. Cell, 11: 819-831, 2000.
42. Zanetta, J. P., Gouyer, V., Maes, E., Pons, A., Hemon, B., Zweibaum, A., Delannoy, P., and Huet, G. Massive in vitro synthesis of tagged oligosaccharides in 1-benzyl-2-acetamido-2-deoxy-alpha-D-galactopyranoside treated HT-29 cells. Glycobiology, 10: 565-575, 2000.
43. Trombetta, E. S, and Helenius, A. Lectins as chaperones in glycoprotein folding. Curr. Opin. Struct. Biol, 8: 587-592, 1998.
44. Amado, M., Almeida, R., Carneiro, F., Levery, S. B., Holmes, E. H., Nomoto, M., Hollingsworth, M. A., Hassan, H., Schwientek, T., Nielsen, P. A., Bennett, E. P., and Clausen, H. A family of human beta3-galactosyltransferases. Characterization of four members of a UDP-galactose:beta-N-acetyl-glucosamine/beta-nacetyl-galactosamine beta-1,3-galactosyltransferase family. J. Biol. Chem., 273: 12770-12778, 1998.
45. Amado, M., Almeida, R., Schwientek, T., and Clausen, H. Identification and characterization of large galactosyltransferase gene families: galactosyltransferases for all functions. Biochim. Biophys. Acta, 1473: 35-53, 1999.
46. Dohi, T., Yuyama, Y., Natori, Y., SMITH, P. L., Lowe, J. B., and Oshima, M. Detection of N-acetylgalactosaminyltransferase mRNA which determines expression of Sda blood group carbohydrate structure in human gastrointestinal mucosa and cancer. Int. J. Cancer, 67: 626-631, 1996.
47. Colley, K. J. Golgi localization of glycosyltransferases: more questions than answers. Glycobiology, 7: 1-13, 1997.
48. Schwientek, T. J., Bennett, E. P., Flores, C., Thacker, J., Hollman, M., Reis, C. A., Behrens, J., Mandel, U., Keck, B., Schafer, M. A., Hazelmann, K., Zubarev, R., Roepstorff, P., Hollingsworth, M. A., and Clausen, H. Functional conservation of subfamilies of putative UDP-N-acetylgalactosamine:Polypeptide N-acetylgalactosaminyltransferases in drosophila, C. elegans and mammals: One subfamily comprised of 1(2)35Aa is essential in drosophila. J. Biol. Chem., 2002.
49. Jorgensen, C. S., Heegaard, N.H., Holm, A., Hojrup, P., and Houen, G. Polypeptide binding properties of the chaperone calreticulin. Eur. J. Biochem., 267: 2945-2954, 2000.
50. Mandel, U., Hassan, H., Therkildsen, M. H., Rygaard, J., Jacobsen, M., Juhl, B. R., Dabelsteen, E., and Clausen, H. Expression of polypeptide GalNAc-transferases in stratified epithelia and squamous cell carcinomas: immunohistological evaluation using monoclonal antibodies to three members of the GalNAc-transferase family. Glycobiology, 9: 43-52, 1999.
51. Burdick, M. D., Harris, A., Reid, C. J., Iwamura, T., and Hollingsworth, M. A. Oligosaccharides expressed on MUC1 produced by pancreatic and colon tumor cell lines. J. Biol. Chem., 272: 24198-24202, 1997.
52. Girling, A., Bartkova, J., Burchell, J., Gendler, S., Gillett, C., and Taylor-Papadimitriou, J. A core protein epitope of the polymorphic epithelial mucin detected by the monoclonal antibody SM-3 is selectively exposed in a range of primary carcinomas. International Journal of Cancer, 43: 1072-1076, 1989.
53. Reis, C. A., Hassan, H., Bennett, E. P., and Clausen, H. Characterization of a panel of monoclonal antibodies using GalNAc glycosylated peptides and recombinant MUC1. Tumour Biology, 19 Suppl 1: 127-133, 1998.
54. Ryuko, K., Schol, D. J., Snijdewint, F. G., Mensdorff-Pouilly, S., Poort-Keesom, R. J., Karuntu-Wanamarta, Y. A., Verstraeten, R. A., Miyazaki, K., Kenemans, P., and Hilgers, J. Characterization of a new MUC1 monoclonal antibody (VU-2-G7) directed to the glycosylated PDTR sequence of MUC1. Tumour. Biol., 21: 197-210, 2000.
55. Mandel, U., Petersen, 0. W., Sorensen, H., Vedtofte, P., Hakomori, S., Clausen, H., and Dabelsteen, E. Simple mucin-type carbohydrates in oral stratified squamous and salivary gland epithelia. Journal of Investigative Dermatology, 97: 713-721, 1991.
56. Sasaki, H., Bothner, B., Dell, A., and Fukuda, M. Carbohydrate structure of erythropoietin expressed in Chinese hamster ovary cells by a human erythropoietin cDNA. J. Biol. Chem., 262: 12059-12076, 1987.
57. Kuan, S. F., Byrd, J. C., Basbaum, C., and Kim, Y. S. Inhibition of mucin glycosylation by aryl-N-acetyl-alpha-galactosaminides in human colon cancer cells. J. Biol. Chem., 264: 19271-19277, 1989.
58. Ramaakrishnan, B., Balaji, P. V., and Qasba, P. K. Crystal Structure of β-1,4-Galactosyltransferase Complex with UDP-Gal Reveals an Oligosaccharide Acceptor Binding Site. J. Mol. Biol., 318:491-502, 2002.
59. Ramalcrishnan, B., and Qasba, P. K. Structure-based Design of β4Gal-T1 with Equally Efficient N-Acetylgalactosaminyltransferase Activity. J. Bio. Chem., 277, 23, 20833-20839, 2002.
60. Ulloa F., and Real, FX. Benzyl-N-acetyl-alpha-D-galactosaminide induces a storage disease-like phenotype by perturbing the endocytic pathway. J Biol. Chem. 2003; 278:12374-83.
61. Leteurtre E, Gouyer V, Delacour D, Hemon B, Pons A, Richet C, Zanetta J P, Huet G. Induction of a storage phenotype and abnormal intracellular localization of apical glycoproteins are two independent responses to GalNAcalpha-O-bn. J Histochem Cytochem. 2003; 51:349-61.

1-43. (canceled)

44. An isolated lectin polypeptide consisting of a truncated mammalian UDP-GalNAc:polypeptide N-acetylgalactosaminyltransferase comprising a domain having an amino acid sequence selected from the group consisting of the amino acid sequences of SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, and SEQ ID NO: 127, wherein:
(i) the lectin domain has lectin binding activity; and
(ii) the truncated polypeptide does not encompass the intact, functioning catalytic domain of the enzyme.

45. An isolated lectin polypeptide consisting of a truncated mammalian UDP-GalNAc:polypeptide N-acetylgalactosaminyltransferase comprising a domain having an amino acid sequence of SEQ ID NO: 99, wherein:
(i) the lectin domain has lectin binding activity; and
(ii) the truncated polypeptide does not encompass the intact, functioning catalytic domain of the enzyme.

46. An isolated lectin polypeptide consisting of a truncated mammalian UDP-GalNAc:polypeptide N-acetylgalactosaminyltransferase comprising a domain having an amino acid sequence of SEQ ID NO: 103, wherein:
(i) the lectin domain has lectin binding activity; and
(ii) the truncated polypeptide does not encompass the intact, functioning catalytic domain of the enzyme.

1. An isolated nucleic acid molecule comprising a nucleic acid sequence encoding a polypeptide selected from the group consisting of the lectin domain of a mammalian polypeptide GalNAc-transferase, and a lectin-functional variant or fragment of said lectin domain, wherein said polypeptide does not encompass the intact, functioning catalytic domain of the enzyme.

2. A nucleic acid molecule according to claim 1 comprising a nucleic acid sequence selected from the group consisting of the nucleic acid sequences encoding the GalNAc-T1 to -T16 lectin domains set forth in Table III herein and lectin-functional variants and fragments thereof.

3. The nucleic acid of claim 2 further comprising 30-60 nucleotides of the corresponding GalNAc-transferase sequence at its 5′ or 3′ end.

4. The nucleic acid of claim 1 wherein the polypeptide GalNAc-transferase or lectin-functional variant or fragment of said lectin domain is human.

5. An isolated lectin polypeptide comprising the lectin domain of a mammalian polypeptide GalNAc-transferase or a lectin-functional variant or fragment thereof.

6. A lectin polypeptide according to claim 5 having an amino acid sequence selected from the group consisting of the amino acid sequences of GalNAc-T1 to -T16 set forth in Table III herein and lectin-functional variants and fragments thereof.

7. The polypeptide of claim 6 further comprising 10-20 amino acid residues of the corresponding GalNAc-transferase sequence at its carboxy or amino terminus.

8. The polypeptide of claim 5 wherein the polypeptide GalNAc-transferase or a lectin-functional variant or fragment thereof is human.

9. A method of producing a lectin polypeptide comprising the lectin domain of a mammalian polypeptide GalNAc-transferase or a lectin-functional variant or fragment thereof, said polypeptide not encompassing the intact, functional catalytic domain of said transferase, the method comprising:
(i) growing a host cell transfected with a nucleic acid sequence encoding the lectin domain of a mammalian polypeptide GalNAc-transferase or a lectin-functional variant or fragment of said lectin domain and excluding the intact catalytic domain of the enzyme under conditions suitable for lectin expression; and
(ii) isolating the lectin polypeptide produced by the host cell

10. A method according to claim 9 wherein said nucleic acid sequence is selected from the group consisting of the sequences encoding the GalNAc-T1 to -T16 lectin domains stated in Table III herein and lectin-functional variants and fragments thereof.

11. The method of claim 9 wherein the polypeptide GalNAc-transferase or lectin-functional variant or fragment of said lectin domain is human.

12. A method of identifying a substance that binds to a polypeptide GalNAc-transferase lectin domain, which comprises
(i) reacting a lectin polypeptide according to claim 5 with at least one substance which potentially may bind to the polypeptide, under conditions which permit the association between the substance and the polypeptide;
(ii) removing and/or detecting the polypeptide with associated substance which, if present, indicates that the substance binds to the polypeptide.

13. A method of screening for inhibitors of functions mediated by polypeptide GalNAc-transferase lectin domains which comprises using a lectin polypeptide according to claim 5 in a binding assay where it interacts with a GalNAc or Galβ1-3GalNAc O-glycopeptide ligand or a molecular mimic hereof, and measuring the binding inhibition to identify and evaluate efficiency of a potential inhibitor.

14. A method of screening for inhibitors of functions mediated by polypeptide GalNAc-transferase lectin domains which comprises using a polypeptide GalNAc-transferase or a fragment thereof retaining functional lectin binding in a binding assay where it interacts with a GalNAc or Galβ1-3GalNAc O-glycopeptide ligand or a molecular mimic hereof, while the binding capacity of the catalytic domain is inactivated by the presence of EDTA or the absence of UDP or UDP-GalNAc or Mn⁺⁺ or other divalent metal ion, and measuring the binding inhibition to identify and evaluate efficiency of a potential inhibitor.

15. A compound that binds to the lectin domain of a member of the mammalian family of polypeptide GalNAc-transferases and inhibits the binding of a carbohydrate to said domain, wherein said compound does not serve as a substrate for core 1 β1,3-galactosyltransferase activity or other glycosyltransferases acting in mucin O-glycosylation.

16. The compound of claim 15 wherein said family of polypeptide GalNAc-transferases is human.

17. An inhibitor of polypeptide GalNAc-transferase lectin-mediated functions that selectively binds to the lectin domain of said transferase and does not serve as an acceptor substrate for core 1 β1,3-galactosyltransferase or other glycosyltransferases functioning in O-glycosylation.

18. An inhibitor according to claim 17, which is GalNAcβ1-R.

19. An inhibitor according to claim 18 wherein R represents an aglycone.

20. An inhibitor according to claim 18 wherein R represents an aryl group.

21. An inhibitor according to claim 18 wherein R is selected from the group consisting of benzyl, phenyl, p-nitrophenyl, umbelliferyl, and naphtalenemethanol.

22. A method of inhibiting mucin secretion in a subject comprising administering an effective amount of a compound that binds to one or more lectin domains of members of a mammalian family of polypeptide GalNAc-transferases and inhibit binding of such domains to carbohydrates.

23. A method of inhibiting hypersecretion and accumulation of mucin in the lungs of a mammal suffering from a chronic obstructive respiratory pulmonary disease comprising administering to said mammal an effective amount of at least one agent that inhibits the binding of polypeptide GalNAc-transferase lectin domains to GalNAc-glycopeptides, wherein said agent is selected from the group consisting of GalNAcβ1-benzyl, a carbohydrate portion of GalNAcβ1-benzyl, a glycoconjugate that includes a carbohydrate portion of GalNAcβ1-benzyl or a derivative of either that inhibits the binding of GalNAc-glycopeptides to a GalNAc-transferase lectin domain.

24. The method of claim 23 wherein the agent is a glycoconjugate that includes a carbohydrate portion of GalNAcβ1-benzyl.

25. The method of claim 23 wherein said mammal is a human.

26. A method of inhibiting the secretion of mucin in a patient comprising administering to the patient a therapeutically effective amount of an agent selected from the group consisting of GalNAcβ1-benzyl, a carbohydrate portion of GalNAcβ1-benzyl, a glycoconjugate that includes a carbohydrate portion of GalNAcβ1-benzyl or a derivative of either that inhibits the binding of GalNAc-glycopeptides to a GalNAc-transferase lectin domain.

27. The method of claim 26, which selectively inhibits one or more members of the GalNAc-transferase family without inhibiting other glycosyltransferases selected from the group consisting of core 1 β1,3-galactosyltransferases, α2,6-sialyltransferases, and glycosyltransferases functioning in the O-glycosylation pathway.

28. The method of claim 26 wherein the patient has a disease selected from the group consisting of chronic obstructive pulmonary diseases, asthma, and cystic fibrosis.

29. A method of modulating the function of one or more lectin domains of a polypeptide GalNAc-transferase comprising administering an effective amount of GalNAcβ1-R which is effective in modulating functions mediated by said lectin domains.

30. The method of claim 29 wherein R represents an aglycone.

31. The method of claim 29 wherein R represents an aryl group.

32. The method of claim 30 wherein R is selected from the group consisting of benzyl, phenyl, p-nitrophenyl, umbelliferyl, and naphtalenemethanol.

33. A method of screening one or more test substances for the ability to inhibit or modulate intracellular transport and/or cell surface expression of mucins, O-glycosylated glycoproteins, glycoproteins and proteins in a cell-based assay, which comprises:
(i) contacting a cell that expresses mucins, O-glycosylated glycoproteins, glycoproteins and proteins, with one or more test substances under assay conditions suitable for the detection of inhibition or modulation of said expression; and
(ii) measuring whether intracellular transport and cell surface expression of said mucins, O-glycosylated glycoproteins, glycoproteins and proteins are thereby inhibited or modulated by one or more of the substances.

34. A method of screening one or more test substances for the ability to inhibit or modulate secretions of mucins, O-glycosylated glycoproteins, glycoproteins and proteins in a cell-based assay, which comprises:
(i) contacting a cell that secretes mucins, O-glycosylated glycoproteins, glycoproteins with one or more test substances under assay conditions suitable for the detection of inhibition or modulation of said secretion; and
(ii) measuring whether secretion of said mucins, O-glycosylated glycoproteins, glycoproteins and proteins are thereby inhibited or modulated by one or more of the substances.

35. The method of claim 22, wherein the compound is GalNAcβ1-benzyl.

36. The method of claim 23, wherein the compound is GalNAcβ1-benzyl.

37. The method of claim 23, wherein the compound is GalNAcβ1-benzyl.

38. The method of claim 34, wherein step (ii) further comprises measuring whether the intracellular accumulation of said mucins, O-glycosylated glycoproteins and proteins is inhibited or modulated.

39. A method of inhibiting mucin secretion in a cell comprising delivering to a cell an effective amount of a compound that binds to one or more lectin domains of members of a mammalian family of polypeptide GalNAc-transferases and inhibit binding of such domains to carbohydrates.

40. The method of claim 39, wherein the compound is GalNAcβ1-benzyl.


SEQUENCE LISTING (127 sequences)

SEQ ID NO: 1 - protein, 20 aa, Artificial Sequence
  synthetic peptide
HGVTSAPDTRPAPGSTAPPA

SEQ ID NO: 2 - protein, 24 aa, Artificial Sequence
  synthetic peptide
TAPPAHGVTSAPDTRPAPGSTAPP

SEQ ID NO: 3 - protein, 167 aa, Homo sapiens
YGDISSRVGLRHKLQCKPFSWYLENIYPDSQIPRHYFSLGEIRNVETNQCLDNMARKENEKVGIFNCHGMGGNQVFSYTANKEIRTDDLCLDVSKLNGPVTMLKCHHLKGNQLWEYDPVKLTLQHVNSNQCLDKATEEDSQVPSIRDCNGSRSQQWLLRNVTLPEIF

SEQ ID NO: 4 - protein, 164 aa, Homo sapiens
YGNIQSRLELRKKLSCKPFKWYLENVYPELRVPDHQDIAFGALQQGTNCLDTLGHFADGVVGVYECHNAGGNQEWALTKEKSVKHMDLCLTVVDRAPGSLIKLQGCRENDSRQKWEQIEGNSKLRHVGSNLCLDSRTAKSGGLSVEVCGPALSQQWKFTLNLQQ

SEQ ID NO: 5 - protein, 167 aa, Homo sapiens
FGDLSKRFEIKHRLRCKNFTWYLNNIYPEVYVPDLNPVISGYIKSVGQPLCLDVGENNQGGKPLIMYTCHGLGGNQYFEYSAQHEIRHNIQKELCLHAAQGLVQLKACTYKGHKTVVTGEQIWEIQKDQLLYNPFLKMCLSANGEHPSLVSCNPSDPLQKWILSQND

SEQ ID NO: 6 - protein, 174 aa, Homo sapiens
AYGDISERKLLRERLRCKSFDWYLKNVFPNLHVPEDRPGWHGAIRSRGISSECLDYNSPDNNPTGANLSLFGCHGQGGNQFFEYTSNKEIRFNSVTELCAEVPEQKNYVGMQNCPKDGFPVPANIIWHFKEDGTIFHPHSGLCLSAYRTPEGRPDVQMRTCDALDKNQIWSFEK

SEQ ID NO: 7 - protein, 168 aa, Homo sapiens
DVGNLTQQRELRKKLKCKSFKWYLENVFPDLRAPIVRASGVLINVALGKCISIENTTVILEDCDGSKELQQFNYTWLRLIKCGEWCIAPIPDKGAVRLHPCDNRNKGLKWLHKSTSVFHPELVNHIVFENNQQLLCLEGNFSQKILKVAACDPVKPYQKWKFEKYYEA

SEQ ID NO: 8 - protein, 165 aa, Homo sapiens
SFGDISERLQLREQLHCHNFSWYLHNVYPEMFVPDLTPTFYGAIKNLGTNQCLDVGENNRGGKPLIMYSCHGLGGNQYFEYTTQRDLRHNIAKQLCLHVSKGALGLGSCHFTGKNSQVPKDEEWELAQDQLIRNSGSGTCLTSQDKKPAMAPCNPSDPHQLWLFV

SEQ ID NO: 9 - protein, 166 aa, Homo sapiens
YGDISELKKFREDHNCQSFKWFMEEIAYDITSHYPLPPKNVDWGEIRGFETAYCIDSMGKTNGGFVELGPCHRMGGNQLFRINEANQLMQYDQCLTKGADGSKVMITHCNLNEFKEWQYFKNLHRFTHIPSGKCLDRSEVLHQVFISNCDSSKTTQKWEMNNIHSV

SEQ ID NO: 10 - protein, 179 aa, Homo sapiens
FGDVSSRMALREKLKCKTFDWYLKNVYPLLKPLHTIVGYGRMKNLLDENVCLDQGPVPGNTPIMYYCHEFSSQNVYYHLTGELYVGQLIAEASASDRCLTDPGKAEKPTLEPCSKAAKNRLHIYWDFKPGGAVINRDTKRCLEMKKDLLGSHVLVLQTCSTQVWEIQHTVRDWGQTNSQ

SEQ ID NO: 11 - protein, 177 aa, Homo sapiens
FGDVSERLALRQRLKCRSFKWYLENVYPEMRVYNNTLTYGEVRNSKASAYCLDQGAEDGDRAILYPCHGMSSQLVRYSADGLLQLGPLGSTAFLPDSKCLVDDGTGRMPTLKRCEDVARPTQRLWDFTQSGPIVSRATGRCLEVEMSKDANFGLRLVVQRCSGQKWMIRNWIKHARH

SEQ ID NO: 12 - protein, 187 aa, Homo sapiens
AGDVAVQKKLRSSLNCKSFKWFMTKIAWDLPKFYPPVEPPAAAWGEIRNVGTGLCADTKHGALGSPLRLEGCVRGRGEAAWNNMQVFTFTWREDIRPGDPQHTKKFCFDAISHTSPVTLYDCHSMKGNQLWKYRKDKTLYHPVSGSCMDCSESDHRIFMNTCNPSSLTQQWLFEHTNSTVLEKFNRN

SEQ ID NO: 13 - protein, 181 aa, Homo sapiens
NISERVELRKKLGCKSFKWYLDNVYPEMQISGSHAKPQQPIFVNRGPKRPKVLQRGRLYHLQTNKCLVAQGRPSQKGGLVVLKACDYSDPNQIWIYNEEHELVLNSLLCLDMSETRSSDPPRLMKCHGSGGSQQWTFGKNNRLYQVSVGQCLRAVDPLGQKGSVAMAICDGSSSQQWHLEG

SEQ ID NO: 14 - protein, 173 aa, Homo sapiens
DVTERKQLRDKLQCKDFKWFLETVYPELHVPEDRPGFFGMLQNKGLTDYCFDYNPPDENQIVGHQVILYLCHGMGQNQFFEYTSQKEIRYNTHQPEGCIAVEAGMDTLIMHLCEETAPENQKFILQEDGSLFHEQSKKCVQAARKESSDSFVPLLRDCTNSDHQKWFFKERML

SEQ ID NO: 15 - protein, 174 aa, Homo sapiens
EKPDCMERLQLQRRLGCRTFHWFLANVYPELYPSEPRPSFSGKLHNTGLGLCADCQAEGDILGCPMVLAPCSDSRQQQYLQHTSRKEIHFGSPQHLCFAVRQEQVILQNCTEEGLAIHQQHWDFQENGMIVHILSGKCMEAVVQENNKDLYLRPCDGKARQQWRFDQINAVDER

SEQ ID NO: 16 - protein, 165 aa, Homo sapiens
YGDVSVRKTLRENLKCKPFSWYLENIYPDSQIPRRYYSLGEIRNVETNQCLDNMGRKENEKVGIFNCHGMGGNQVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 17 - protein, 171 aa, Homo sapiens
FGNVESRLDLRKNLRCQSFKWYLENIYPELSIPKESSIQKGNIRQRQKCLESQRQNNQETPNLKLSPCAKVKGEDAKSQVWAFTYTQKILQEELCLSVITLFPGAPVVLVLCKNGDDRQQWTKTGSHIEHIASHLCLDTDMFGDGTENGKEIGVNPCESSLMSQHWDMVSS

SEQ ID NO: 18 - protein, 163 aa, Homo sapiens
SVATRIEQRKKMNCKSFRWYLENVYPELTVPVKEALPGIIKQGVNCLESQGQNTAGDFLLGMGICRGSAKNPQPAQAWLFSDHLIQQQGKCLAATSTLMSSPGSPVILQMCNPREGKQKWRRKGSFIQHSVSGLCLETKPAQLVTSKCQADAQAQQWQLLPHT

SEQ ID NO: 19 - protein, 8 aa, Homo sapiens
WYLENVYP

SEQ ID NO: 20 - DNA, 29 bp, Artificial Sequence
  PCR primer
gcgggatccaggacttcctgctggagatg

SEQ ID NO: 21 - DNA, 28 bp, Artificial Sequence
  PCR primer
gcggatcctcagaatatttctggaaggg

SEQ ID NO: 22 - DNA, 35 bp, Artificial Sequence
  PCR primer
gcggaattcttaaaaagaaagaccttcatcacagc

SEQ ID NO: 23 - DNA, 28 bp, Artificial Sequence
  PCR primer
gcggaattcctactgctgcaggttgagc

SEQ ID NO: 24 - DNA, 28 bp, Artificial Sequence
  PCR primer
gcgggatccaacgatggaaaggaacatg

SEQ ID NO: 25 - DNA, 31 bp, Artificial Sequence
  PCR primer
agcggatccaggaacacttaatcattttggc

SEQ ID NO: 26 - DNA, 31 bp, Artificial Sequence
  PCR primer
gcgggatccttttcatgcctccgcaggagcc

SEQ ID NO: 27 - DNA, 30 bp, Artificial Sequence
  PCR primer
gcgggatccgacgaaagtgctgttgtgctc

SEQ ID NO: 28 - DNA, 32 bp, Artificial Sequence
  PCR primer
gcgggatcctgctttaactggagggctagagc

SEQ ID NO: 29 - DNA, 30 bp, Artificial Sequence
  PCR primer
gcgggatccatcagttacacttcaggcttc

SEQ ID NO: 30 - DNA, 34 bp, Artificial Sequence
  PCR primer
gcgggatcccctggacctcatgctggaggccatg

SEQ ID NO: 31 - DNA, 32 bp, Artificial Sequence
  PCR primer
agcggatcctggggatgatctgggtcctagac

SEQ ID NO: 32 - DNA, 32 bp, Artificial Sequence
  PCR primer
gcgaagcttcaggatgagggaagacagagatg

SEQ ID NO: 33 - DNA, 35 bp, Artificial Sequence
  PCR primer
gcgaagcttctctctaaacactatggatcttattc

SEQ ID NO: 34 - DNA, 33 bp, Artificial Sequence
  PCR primer
gcgggatcctctgaaagaaagtatgaaattagc

SEQ ID NO: 35 - DNA, 30 bp, Artificial Sequence
  PCR primer
gcgggatcctcactggctgttggtctgacc

SEQ ID NO: 36 - DNA, 34 bp, Artificial Sequence
  PCR primer
gcgggatccctgccgcctgcagggccgctcccag

SEQ ID NO: 37 - DNA, 32 bp, Artificial Sequence
  PCR primer
gcgggatcctcagtgccgtcggtgtttgatcc

SEQ ID NO: 38 - DNA, 31 bp, Artificial Sequence
  PCR primer
gcgggatccccgcgagcggcagcccgacggc

SEQ ID NO: 39 - DNA, 30 bp, Artificial Sequence
  PCR primer
gcgggatcctcagttcctattgaatttttc

SEQ ID NO: 40 - DNA, 31 bp, Artificial Sequence
  PCR primer
gcgaattcgtgaagtgactcagccacttaag

SEQ ID NO: 41 - DNA, 28 bp, Artificial Sequence
  PCR primer
gcgaattcgtctctgtcagacacgtgtc

SEQ ID NO: 42 - DNA, 32 bp, Artificial Sequence
  PCR primer
gcgggatccggctcggtgctgcgggcgcagcg

SEQ ID NO: 43 - DNA, 35 bp, Artificial Sequence
  PCR primer
gcgggatcctcataacatgcgctctttgaagaacc

SEQ ID NO: 44 - DNA, 34 bp, Artificial Sequence
  PCR primer
gcgggatccgatgttgcacvvtccccaccacacc

SEQ ID NO: 45 - DNA, 31 bp, Artificial Sequence
  PCR primer
gcgggatcctcatcgttcatccacagcattg

SEQ ID NO: 46 - DNA, 32 bp, Artificial Sequence
  PCR primer
gcgggatcctctgctgcctgcattgagggctg

SEQ ID NO: 47 - DNA, 32 bp, Artificial Sequence
  PCR primer
gcgggatcctcatgtgcccaaggtcatgttcc

SEQ ID NO: 48 - DNA, 31 bp, Artificial Sequence
  PCR primer
gcgggatcccaagaggaagttggaggtgccg

SEQ ID NO: 49 - DNA, 31 bp, Artificial Sequence
  PCR primer
gcgggatcccaggggtcctcaagagctcacc

SEQ ID NO: 50 - DNA, 33 bp, Artificial Sequence
  PCR primer
gcgggatccctactacttatggcaggacaaccg

SEQ ID NO: 51 - DNA, 30 bp, Artificial Sequence
  PCR primer
gcgtcatgtgtgtggcaacagctgccactg

SEQ ID NO: 52 - DNA, 157 bp, Artificial Sequence
  synthetic nucleotide
gcggccgctctagaactagtggatccagcagccatcatcatcatcatcacagcagcggcctggtgccgcgcggcagccatatggctagcatgactggtggacagcaaatgggtcgcggaattccgatatcaagcttatcgataccgtcgacctcgag

SEQ ID NO: 53 - DNA, 123 bp, Artificial Sequence
  synthetic nucleotide
gaattcgcggccgcagcagccatcatcatcatcatcacagcagcggcctggtgccgcgcggcagccatatggctagcatgactggtggacagcaaatggatccactagttctagagcggccgc

SEQ ID NO: 54 - DNA, 1746 bp, Homo sapiens
atgtgggggcgcacggcgcggcggcgctgcccgcgggaactgcggcgcggccgggaggcgctgttggtgctcctggcgctactggcgttggccgggctgggctcggtgctgcgggcgcagcgtggggccggggccggggctgccgagccgggacccccgcgcaccccgcgccccgggcggcgcgagccggtcatgccgcggccgccggtgccggcgaacgcgctgggcgcgcggggcgaggcggtgcggctgcagctgcagggcgaggagctgcggctgcaggaggagagcgtgcggctgcaccagattaacatctacctcagcgaccgcatctcactgcaccgccgcctgcccgagcgctggaacccgctgtgcaaagagaagaaatatgattatgataatttgcccaggacatctgttatcatagcattttataatgaagcctggtcaactctccttcggacagtttacagtgtccttgagacatccccggatatcctgctagaagaagtgatccttgtagatgactacagtgatagagagcacctgaaggagcgcttggccaatgagctttcgggactgcccaaggtgcgcctgatccgcgccaacaagagagagggcctggtgcgagcccggctgctgggggcgtctgcggcgaggggcgatgttctgaccttcctggactgtcactgtgaatgccacgaagggtggctggagccgctgctgcagaggatccatgaagaggagtcggcagtggtgtgcccggtgattgatgtgatcgactggaacaccttcgaatacctggggaactccggggagccccagatcggcggtttcgactggaggctggtgttcacgtggcacacagttcctgagagggagaggatacggatgcaatcccccgtcgatgtcatcaggtctccaacaatggctggtgggctgtttgctgtgagtaagaaatattttgaatatctggggtcttatgatacaggaatggaagtttggggaggagaaaacctcgaattttcctttaggatctggcagtgtggtggggttctggaaacacacccatgttcccatgttggccatgttttccccaagcaagctccctactcccgcaacaaggctctggccaacagtgttcgtgcagctgaagtatggatggatgaatttaaagagctctactaccatcgcaacccccgtgcccgcttggaaccttttggggatgtgacagagaggaagcagctccgggacaagctccagtgtaaagacttcaagtggttcttggagactgtgtatccagaactgcatgtgcctgaggacaggcctggcttcttcgggatgctccagaacaaaggactaacagactactgctttgactataaccctcccgatgaaaaccagattgtgggacaccaggtcattctgtacctctgtcatgggatgggccagaatcagtttttcgagtacacgtcccagaaagaaatacgctataacacccaccagcctgagggctgcattgctgtggaagcaggaatggatacccttatcatgcatctctgcgaagaaactgccccagagaatcagaagttcatcttgcaggaggatggatctttatttcacgaacagtccaagaaatgtgtccaggctgcgaggaaggagtcgagtgacagtttcgttccactcttacgagactgcaccaactcggatcatcagaaatggttcttcaaagagcgcatgttatga

SEQ ID NO: 55 - protein, 581 aa, Homo sapiens
MWGRTARRRCPRELRRGREALLVLLALLALAGLGSVLRAQRGAGAGAAEPGPPRTPRPGRREPVMPRPPVPANALGARGEAVRLQLQGEELRLQEESVRLHQINIYLSDRISLHRRLPERWNPLCKEKKYDYDNLPRTSVIIAFYNEAWSTLLRTVYSVLETSPDILLEEVILVDDYSDREHLKERLANELSGLPKVRLIRANKKKGLVRARLLGASAARGDVLTFLDCHCECHEGWLEPLLQRIHEEESAVVCPVIDVIDWNTFEYLGNSGEPQIGGFDWRLVFTWHTVPERERIRMQSPVDVIRSPTMAGGLFAVSKKYFEYLGSYDTGMEVWGGENLEFSFRIWQCGGVLETHPCSHVGHFSPSKLPTPRNKALANSVRAAEVWMDEFKELYYHRNPRARLEPFGDVTERKQLRDKLQCKDFKWFLETVYPELHVPEDRPGFFGMLQNKGLTDYCFDYNPPDENQIVGHQVILYLCHGMGQNQFFEYTSQKEIRYNTHQPEGCIAVEAGMDTLIMHLCEETAPENQKFILQEDGSLFHEQSKKCVQAARKESSDSFVPLLRDCTNSDHQKWFFKERML

SEQ ID NO: 56 - DNA, 1920 bp, Homo sapiens
atgctcctaaggaagcgatacaggcacagaccatgcagactccagttcctcctgctgctcctgatgctgggatgcgtcctgatgatggtggcgatgttgcaccctccccaccacaccctgcaccagactgtcacagcccaagccagcaagcacagccctgaagccaggtaccgcctggactttggggaatcccaggattgggtactggaagctgaggatgagggtgaagagtacagccctctggagggcctgccaccctttatctcactgcgggaggatcagctgctggtggccgtggccttaccccaggccagaaggaaccagagccagggcaggagaggtgggagctaccgcctcatcaagcagccaaggaggcaggataaggaagccccaaagagggactggggggctgatgaggacggggaggtgtctgaagaagaggagttgaccccgttcagcctggacccacgtggcctccaggaggcactcagtgcccgcatccccctccagagggctctgcccgaggtgcggcacccactgtgtctgcagcagcaccctcaggacagcctgcccacagccagcgtcatcctctgtttccatgatgaggcctggtccactctcctgcggactgtacacagcatcctcgacacagtgcccagggccttcctgaaggagatcatcctcgtggacgacctcagccagcaaggacaactcaagtctgctctcagcgaatatgtggccaggctggagggggtgaagttactcaggagcaacaagaggctgggtgccatcagggcccggatgctgggggccaccagagccaccggggatgtgctcgtcttcatggatgcccactgcgagtgccacccaggctggctggagcccctcctcagcagaatagctggtgacaggagccgagtggtatctccggtgatagatgtgattgactggaagactttccagtattacccctcaaaggacctgcagcgtggggtgttggactggaagctggatttccactgggaacctttgccagagcatgtgaggaaggccctccagtcccccataagccccatcaggagccctgtggtgcccggagaggtggtggccatggacagacattacttccaaaacactggagcgtatgactctcttatgtcgctgcgaggtggtgaaaacctcgaactgtctttcaaggcctggctctgtggtggctctgttgaaatccttccctgctctcgggtaggacacatctaccaaaatcaggattcccattcccccctcgaccaggaggccaccctgaggaacagggttcgcattgctgagacctggctggggtcattcaaagaaaccttctacaagcatagcccagaggccttctccttgagcaaggctgagaagccagactgcatggaacgcttgcagctgcaaaggagactgggttgtcggacattccactggtttctggctaatgtctaccctgagctgtacccatctgaacccaggcccagtttctctggaaagctccacaacactggacttgggctctgtgcagactgccaggcagaaggggacatcctgggctgtcccatggtgttggctccttgcagtgacagccggcagcaacagtacctgcagcacaccagcaggaaggagattcactttggcagcccacagcacctgtgctttgctgtcaggcaggagcaggtgattcttcagaactgcacggaggaaggcctggccatccaccagcagcactgggacttccaggagaatgggatgattgtccacattctttctgggaaatgcatggaagctgtggtgcaagaaaacaataaagatttgtacctgcgtccgtgtgatggaaaagcccgccagcagtggcgttttgaccagatcaatgctgtggatgaacgatga

SEQ ID NO: 57 - protein, 639 aa, Homo sapiens
MLLRKRYRHRPCRLQFLLLLLMLGCVLMMVAMLHPPHHTLHQTVTAQASKHSPEARYRLDFGESQDWVLEAEDEGEEYSPLEGLPPFISLREDQLLVAVALPQARRNQSQGRRGGSYRLIKQPRRQDKEAPKRDWGADEDGEVSEEEELTPFSLDPRGLQEALSARIPLQRALPEVRHPLCLQQHPQDSLPTASVILCFHDEAWSTLLRTVHSILDTVPRAFLKEIILVDDLSQQGQLKSALSEYVARLEGVKLLRSNKRLGAIRARMLGATRATGDVLVFMDAHCECHPGWLEPLLSRIAGDRSRVVSPVIDVIDWKTFQYYPSKDLQRGVLDWKLDFHWEPLPEHVRKALQSPISPIRSPVVPGEVVAMDRHYFQNTGAYDSLMSLRGGENLELSFKAWLCGGSVEILPCSRVGHIYQNQDSHSPLDQEATLRNRVRIAETWLGSFKETFYKHSPEAFSLSKAEKPDCMERLQLQRRLGCRTFHWFLANVYPELYPSEPRPSFSGKLHNTGLGLCADCQAEGDILGCPMVLAPCSDSRQQQYLQHTSRKEIHFGSPQHLCFAVRQEQVILQNCTEEGLAIHQQHWDFQENGMIVHILSGKCMEAVVQENNKDLYLRPCDGKARQQWRFDQINAVDER

SEQ ID NO: 58 - DNA, 1671 bp, Homo sapiens
atgaggagatttgtctactgcaaggtggttctagccacttcgctgatgtgggttcttgttgatgtcttcttactgctgtacttcagtgaatgtaacaaatgtgatgacaagaaggagagatctctgctgcctgcattgagggctgttatttcaagaaaccaagaagggccaggagaaatgggaaaagctgtgttgattcctaaagatgaccaggagaaaatgaaagagctgtttaaaatcaatcagtttaaccttatggccagtgatttgattgcccttaatagaagtctgccagatgtaagattagaaggatgtaagacaaaagtctaccctgatgaacttccaaacacaagtgtagtcattgtgtttcataatgaagcttggagcactctccttagaactgtttacagtgtgataaatcgttccccacactatctactctcagaggtcatcttggtagatgatgccagtgaaagagattttctcaagttgacattagagaattacgtgaaaaatttagaagtgccagtaaaaattattaggatggaagaacgctctgggttaatacgtgcccgtcttcgaggagcagctgcttcaaaagggcaggtcataacttttcttgatgcacactgtgaatgcacgttaggatggctggagcctttgctggcaagaataaaggaagacaggaaaacggttgtctgccctatcattgatgtgattagtgatgatacttttgaatatatggctgggtcagacatgacttatgggggttttaactggaaactgaatttccgctggtatcctgttccccaaagagaaatggacaggaggaaaggagacagaacattacctgtcaggacccctactatggctggtggcctattttctattgacagaaactactttgaagagataggaacttacgatgcaggaatggatatctggggtggagagaatcttgaaatgtcttttaggatttggcaatgtggaggctccttggagattgttacttgctcccatgttggtcatgtttttcggaaggcaactccatacacttttcctggtggcactggtcatgtcatcaacaagaacaacaggagactggcagaagtttggatggatgaatttaaagatttcttctacatcatatccccaggtgttgtcaaagtggattatggagatgtgtcagtcagaaaaacactaagagaaaatctgaagtgtaagcccttttcttggtacctagaaaacatctatccggactcccagatcccaagacgttattactcacttggtgagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 59 - protein, 556 aa, Homo sapiens
MRRFVYCKVVLATSLMWVLVDVFLLLYFSECNKCDDKKERSLLPALRAVISRNQEGPGEMGKAVLIPKDDQEKMKELFKINQFNLMASDLIALNRSLPDVRLEGCKTKVYPDELPNTSVVIVFHNEAWSTLLRTVYSVINRSPHYLLSEVILVDDASERDFLKLTLENYVKNLEVPVKIIRMEERSGLIRARLRGAAASKGQVITFLDAHCECTLGWLEPLLARIKEDRKTVVCPIIDVISDDTFEYMAGSDMTYGGFNWKLNFRWYPVPQREMDRRKGDRTLPVRTPTMAGGLFSIDRNYFEEIGTYDAGMDIWGGENLEMSFRIWQCGGSLEIVTCSHVGHVFRKATPYTFPGGTGHVINKNNRRLAEVWMDEFKDFFYIISPGVVKVDYGDVSVRKTLRENLKCKPFSWYLENIYPDSQIPRRYYSLGEIRNVETNQCLDNMGRKENEKVGIFNCHGMGGNQVFSYTADKEIRTDDLCLDVSRLNGPVIMLKCHHMRGNQLWEYDAERLTLRHVNSNQCLDEPSEEDKMVPTMQDCSGSRSQQWLLRNMTLGT

SEQ ID NO: 60 - DNA, 1911 bp, Homo sapiens
atgcggcgcctgactcgtcggctggttctgccagtcttcggggtgctctggatcacggtgctgctgttcttctgggtaaccaagaggaagttggaggtgccgacgggacctgaagtgcagacccctaagccttcggacgctgactgggacgacctgtgggaccagtttgatgagcggcggtatctgaatgccaaaaagtggcgcgttggtgacgacccctataagctgtatgctttcaaccagcgggagagtgagcggatctccagcaatcgggccatcccggacactcgccatctgagatgcacactgctggtgtattgcacggaccttccacccactagcatcatcatcaccttccacaacgaagcccgctccacgctgctcaggaccatccgcagtgtattaaaccgcacccctacgcatctgatccgggaaatcatattagtggatgacttcagcaatgaccctgatgactgtaaacagctcatcaaattgcccaaggtgaaatgcttgcgcaataatgaacggcaaggtctggtccggtcccggattcggggcgctgacatcgcccagggcaccactctgactttcctcgacagccactgtgaggtgaacagggactggctccagcctctgttgcacagggtcaaagaagactacacgcgggtggtgtgccctgtgatcgatatcattaacctggacaccttcacctacatcgagtctgcctcggagctcagaggggggtttgactggagcctccacttccagtgggagcagctctccccagagcagaagctcggcgcctggaccccacggaagcccatcaggactcctatcatagctggagggctcttcgtgatcgacaaagcttggtttgattacctggggaaatatgatatggacatggacatctggggtggggagaactttgaaatctccttccgagtgtggatgtgcgggggcagcctagagatcgtcccctgcagccgagtggggcacgtcttccggaagaagcacccctacgttttccctgatggaaatgccaacacgtatataaagaacaccaagcggacagctgaagtgtggatggatgaatacaagcaatactattacgctgcccggccattcgccctggagaggcccttcgggaatgttgagagcagattggacctgaggaagaatctgcgctgccagagcttcaagtggtacctggagaatatctaccctgaactcagcatccccaaggagtcctccatccagaagggcaatatccgacagagacagaagtgcctggaatctcaaaggcagaacaaccaagaaaccccaaacctaaagttgagcccctgtgccaaggtcaaaggcgaagatgcaaagtcccaggtatgggccttcacatacacccagcagatcctccaggaggagctgtgcctgtcagtcatcaccttgttccctggcgccccagtggttcttgtcctttgcaagaatggagatgaccgacagcaatggaccaaaactggttcccacatcgagcacatagcatcccacctctgcctcgatacagatatgttcggtgatggcaccgagaacggcaaggaaatcgtcgtcaacccatgtgagtcctcactcatgagccagcactgggacatggtgagctcttgaggacccctgccagaagcagcaagggccatggggtggtgcttccctggaccagaacagactggaaactgggcagcaagcagcctgcaaccacctcagacatcctggactgggaggtggaggcagagccccccaggacaggagcaactgtctcagggaggacagaggaaaacatcacaagccaatggggctcaaagacaaatcccacatgttctcaaggccgttaagttccagtcctggccagtcattccctga

SEQ ID NO: 61 - protein, 552 aa, Homo sapiens
MRRLTRRLVLPVFGVLWITVLLFFWVTKRKLEVPTGPEVQTPKPSDADWDDLWDQFDERRYLNAKKWRVGDDPYKLYAFNQRESERISSNRAIPDTRHLRCTLLVYCTDLPPTSIIITFHNEARSTLLRTIRSVLNRTPTHLIREIILVDDFSNDPDDCKQLIKLPKVKCLRNNERQGLVRSRIRGADIAQGTTLTFLDSHCEVNRDWLQPLLHRVKEDYTRVVCPVIDIINLDTFTYIESASELRGGFDWSLHFQWEQLSPEQKLGAWTPRKPIRTPIIAGGLFVIDKAWFDYLGKYDMDMDIWGGENFEISFRVWMCGGSLEIVPCSRVGHVFRKKHPYVFPDGNANTYIKNTKRTAEVWMDEYKQYYYAARPFALERPFGNVESRLDLRKNLRCQSFKWYLENIYPELSIPKESSIQKGNIRQRQKCLESQRQNNQETPNLKLSPCAKVKGEDAKSQVWAFTYTQQILQEELCLSVITLFPGAPVVLVLCKNGDDRQQWTKTGSHIEHIASHLCLDTDMFGDGTENGKEIVVNPCESSLMSQHWDMVSS

SEQ ID NO: 62 - DNA, 1677 bp, Homo sapiens
atgaggaagatccgcgccaatgccatcgccatcctgaccgtagcctggatcctgggcactttctactacttatggcaggacaaccgagcccacgcagcatcctccggcggccggggcgcgcagagggcaggcaggaggtcggagcagctccgcgaggaccgcaccatcccgctcattgtgacaggaactccctcgaaaggctttgatgagaaggcctacctgtcggccaagcagctgaaggctggagaggacccctacagacagcacgccttcaaccagctggagagtgacaagctgagcccagaccggcccatccgggacacccgccattacagctgcccatctgtgtcctactcctcggacctgccagccaccagcgtcatcatcaccttccacaatgaggcccgttccaccctgctgcgcacagtgaagagtgtcctgaaccgaactcctgccaacttgatccaggagatcattttagtggatgacttcagctcagatccggaagactgtctactcctgaccaggatccccaaggtcaagtgcctgcgcaatgatcggcgggaagggctgatccggtcccgagtgcgtggggcggacgtggctgcagctaccgttctcacctttctggatagccactgcgaagtgaacaccgagtggctgccgcccatgctgcagcgggtgaaggaggaccacacccgcgtggtgagtcccatcattgatgtcatcagtctggataattttgcctaccttgcagcatctgctgaccttcgtggagggttcgactggagcctgcatttcaagtgggagcagatccctcttgagcagaagatgacccggacagaccccaccaggcccataaggacgcctgtcatagctggaggaatcttcgtgatcgacaagtcctggtttaaccacttgggaaagtatgatgcccagatggacatctgggggggagagaattttgagctctccttcagggtgtggatgtgtggtggcagtctggagatcgtcccctgcagccgggtgggccatgtcttcaggaaacggcacccctacaacttccctgagggtaatgccctcacctacatcaggaatactaagcgcactgcagaagtgtggatggatgaatacaagcaatactactatgaggcccggccctcggccatcgggaaggccttcggcagtgtggctacgcggatagagcagaggaagaagatgaactgcaagtccttccgctggtacctggagaacgtctacccagagctcacggtccccgtgaaggaagcactccccggcatcattaagcagggggtgaactgcttagaatctcagggccagaacacagctggtgacttcctgcttggaatggggatctgcagagggtctgccaagaacccgcagcccgcccaggcatggctgttcagtgaccacctcatccagcagcaggggaagtgcctggctgccacctccaccttaatgtcctcccctggatccccagtcatactgcagatgtgcaaccctagagaaggcaagcagaaatggaggagaaaaggatctttcatccagcattcagtcagtggcctctgcctggagacaaagcctgcccagctggtgaccagcaagtgtcaggctgacgcccaggcccagcagtggcagctgttgccacacacatga

SEQ ID NO: 63 - protein, 558 aa, Homo sapiens
MRKIRANAIAILTVAWILGTFYYLWQDNRAHAASSGGRGAQRAGRRSEQLREDRTIPLIVTGTPSKGFDEKAYLSAKQLKAGEDPYRQHAFNQLESDKLSPDRPIRDTRHYSCPSVSYSSDLPATSVIITFHNEARSTLLRTVKSVLNRTPANLIQEIILVDDFSSDPEDCLLLTRIPKVKCLRNDRREGLIRSRVRGADVAAATVLTFLDSHCEVNTEWLPPMLQRVKEDHTRVVSPIIDVISLDNFAYLAASADLRGGFDWSLHFKWEQIPLEQKMTRTDPTRPIRTPVIAGGIFVIDKSWFNHLGKYDAQMDIWGGENFELSFRVWMCGGSLEIVPCSRVGHVFRKRHPYNFPEGNALTYIRNTKRTAEVWMDEYKQYYYEARPSAIGKAFGSVATRIEQRKKMNCKSFRWYLENVYPELTVPVKEALPGIIKQGVNCLESQGQNTAGDFLLGMGICRGSAKNPQPAQAWLFSDHLIQQQGKCLAATSTLMSSPGSPVILQMCNPREGKQKWRRKGSFIQHSVSGLCLETKPAQLVTSKCQADAQAQQWQLLPHT

SEQ ID NO: 64 - DNA, 33 bp, Artificial Sequence
  PCR primer
caaaggaagcttatggagatatatcgtcaagag

SEQ ID NO: 65 - DNA, 43 bp, Artificial Sequence
  PCR primer
gcaagctcgaggcggccgctcagaatatttctggaagggtgac

SEQ ID NO: 66 - DNA, 37 bp, Artificial Sequence
  PCR primer
caaggaagcttcttatggaaatattcagagcagattg

SEQ ID NO: 67 - DNA, 38 bp, Artificial Sequence
  PCR primer
gcaagctcgaggcggccgcctactgctgcaggttgagc

SEQ ID NO: 68 - DNA, 37 bp, Artificial Sequence
  PCR primer
caaggaagcttcatttggtgatctttcaaaaagattt

SEQ ID NO: 69 - DNA, 40 bp, Artificial Sequence
  PCR primer
gcaagctcgaggcggccgcaggaacacttaatcattttgg

SEQ ID NO: 70 - DNA, 28 bp, Artificial Sequence
  PCR primer
agaaaagaagcttatggtgatatttctg

SEQ ID NO: 71 - DNA, 28 bp, Artificial Sequence
  PCR primer
agcggatccgacgaagtgctgttgtgct

SEQ ID NO: 72 - DNA, 35 bp, Artificial Sequence
  PCR primer
caaggaagctttagatgttggcaacctcacccagc

SEQ ID NO: 73 - DNA, 44 bp, Artificial Sequence
  PCR primer
gcaagctcgaggcggccgcaagcatcagttacacttcaggcttc

SEQ ID NO: 74 - DNA, 33 bp, Artificial Sequence
  PCR primer
caaggaagcttccttcggtgacatttcggaacg

SEQ ID NO: 75 - DNA, 39 bp, Artificial Sequence
  PCR primer
gcaagctcgaggcggccgctgggtcctagacaaagagcc

SEQ ID NO: 76 - DNA, 33 bp, Artificial Sequence
  PCR primer
agaaaagaagcttatggggatatatcggagctg

SEQ ID NO: 77 - DNA, 44 bp, Artificial Sequence
  PCR primer
gcaagctcgaggcggccgctctctaaacactatggatgttattc

SEQ ID NO: 78 - DNA, 34 bp, Artificial Sequence
  PCR primer
caaggaagcttttggagacgtttcttccagaatg

SEQ ID NO: 79 - DNA, 42 bp, Artificial Sequence
  PCR primer
gcaagctcgaggcggccgctcactggctgttggtctgacccc

SEQ ID NO: 80 - DNA, 34 bp, Artificial Sequence
  PCR primer
caaggaagctttcggggacgtgtctgagaggctg

SEQ ID NO: 81 - DNA, 42 bp, Artificial Sequence
  PCR primer
gcaagctcgaggcggccgctcagtgccgtgcgtgtttgatcc

SEQ ID NO: 82 - DNA, 34 bp, Artificial Sequence
  PCR primer
caaggaagcttccgctggggatgtcgcagtccag

SEQ ID NO: 83 - DNA, 41 bp, Artificial Sequence
  PCR primer
gcaagctcgaggcggccgctcagttcctattgaatttttcc

SEQ ID NO: 84 - DNA, 32 bp, Artificial Sequence
  PCR primer
caaggaagcttgcaatatcagtgagcgtgtgg

SEQ ID NO: 85 - DNA, 40 bp, Artificial Sequence
  PCR primer
gcaagctcgaggcggccgcccaccttaaccttccaaatgc

SEQ ID NO: 86 - DNA, 31 bp, Artificial Sequence
  PCR primer
caaggaagcttgggatgtgacagagaggaag

SEQ ID NO: 87 - DNA, 45 bp, Artificial Sequence
  PCR primer
gcaagctcgaggcggccgctcataacatgcgctctttgaagaacc

SEQ ID NO: 88 - DNA, 32 bp, Artificial Sequence
  PCR primer
caaggaagcttctgagaagccagactgcatgg

SEQ ID NO: 89 - DNA, 41 bp, Artificial Sequence
  PCR primer
gcaagctcgaggcggccgctcatcgttcatccacagcattg

SEQ ID NO: 90 - DNA, 36 bp, Artificial Sequence
  PCR primer
caaggaagcttatggagatgtgtcagtcagaaaaac

SEQ ID NO: 91 - DNA, 42 bp, Artificial Sequence
  PCR primer
gcaagctcgaggcggccgctcatgtgcccaaggtcatgttcc

SEQ ID NO: 92 - DNA, 34 bp, Artificial Sequence
  PCR primer
caaggaagctttcgggaatgttgagagcagattg

SEQ ID NO: 93 - DNA, 43 bp, Artificial Sequence
  PCR primer
gcaagctcgaggcggccgctcaagaactcaccatgtcccagtg

SEQ ID NO: 94 - DNA, 40 bp, Artificial Sequence
  PCR primer
caaggaagcttgcagtgtggctacgcggatagagcagagg

SEQ ID NO: 95 - DNA, 42 bp, Artificial Sequence
  PCR primer
gcaagctcgaggcggccgctcatgtgtgtggcaacagctgcc

SEQ ID NO: 96 - DNA, 513 bp, Homo sapiens
aaagaagcttatggagatatatcgtcaagagttggtctaagacacaaactacaatgcaaacctttttcctggtacctagagaatatatatcctgattctcaaattccacgtcactatttctcattgggagagatacgaaatgtggaaacgaatcagtgtctagataacatggctagaaaagagaatgaaaaagttggaatttttaattgccatggtatggggggtaatcaggttttctcttatactgccaacaaagaaattagaacagatgacctttgcttggatgtttccaaacttaatggcccagttacaatgctcaaatgccaccacctaaaaggcaaccaactctgggagtatgacccagtgaaattaaccctgcagcatgtgaacagtaatcagtgcctggataaagccacagaagaggatagccaggtgcccagcattagagactgcaatggaagtcggtcccagcagtggcttcttcgaaacgtcacccttccagaaatattctga

SEQ ID NO: 97 - protein, 167 aa, Homo sapiens; identical to SEQ ID NO: 3

SEQ ID NO: 98 - DNA, 417 bp, Homo sapiens
tatccagagttaagggttccagaccatcaggatatagcttttggggccttgcagcagggaactaactgcctcgacactttgggacactttgctgatggtgtggttggagtttatgaatgtcacaatgctgggggaaaccaggaatgggccttgacgaaggagaagtcggtgaagcacatggatttgtgccttactgtggtggaccgggcaccgggctctcttataaagctgcagggctgccgagaaaatgacagcagacagaaatgggaacagatcgagggcaactccaagctgaggcacgtgggcagcaacctgtgcctggacagtcgcacggccaagagcgggggcctaagcgtggaggtgtgtggcccggccctttcgcagcagtggaagttcacgctcaacctgcagcagtag

SEQ ID NO: 99 - protein, 138 aa, Homo sapiens
YPELRVPDHQDIAFGALQQGTNCLDTLGHFADGVVGVYECHNAGGNQEWALTKEKSVKHMDLCLTVVDRAPGSLIKLQGCRENDSRQKWEQIEGNSKLRHVGSNLCLDSRTAKSGGLSVEVCGPALSQQWKFTLNLQQ

SEQ ID NO: 100 - DNA, 507 bp, Homo sapiens
tcatttggtgatctttcaaaaagatttgaaataaaacaccgtcttcggtgtaaaaattttacatggtatctgaacaacatttatccagaggtgtatgtgccagaccttaatcctgttatatctggatacattaaaagcgttggtcagcctctatgtctggatgttggagaaaacaatcaaggaggcaaaccattaattatgtatacatgtcatggacttgggggaaaccagtactttgaatactctgctcaacatgaaattcggcacaacatccagaaggaattatgtcttcatgctgctcaaggtctcgttcagctgaaggcatgtacctacaaaggtcacaagacagttgtcactggagagcagatatgggagatccagaaggatcaacttctatacaatccattcttaaaaatgtgcctttcagcaaatggagagcatccaagtttagtgtcatgcaacccatcagatccactccaaaaatggatacttagccaaaatgattaa

SEQ ID NO: 101 - protein, 167 aa, Homo sapiens; identical to SEQ ID NO: 5

SEQ ID NO: 102 - DNA, 423 bp, Homo sapiens
gaggatagaccaggctggcatggggctattcgcagtagagggatctcgtctgaatgtttagattataattctcctgacaacaaccccacaggtgctaacctttcactgtttggatgccatggtcaaggaggcaatcaattctttgaatatacttcaaacaaagaaataaggtttaattctgtgacagagttatgtgcagaggtacctgagcaaaaaaattatgtgggaatgcaaaattgtcccaaagatgggttccctgtaccagcaaacattatttggcattttaaagaagatggaactatttttcacccacactcaggactgtgtcttagtgcttatcggacaccggagggccgacctgatgtacaaatgagaacttgtgatgctctagataaaaatcaaatttggagttttgagaaatag

SEQ ID NO: 103 - protein, 174 aa, Homo sapiens; identical to SEQ ID NO: 6

SEQ ID NO: 104 - DNA, 510 bp, Homo sapiens
ttagatgttggcaacctcacccagcaaagggagctgcgaaagaaactgaagtgcaaaagtttcaaatggtacttggagaatgtctttcctgacttaagggctcccattgtgagagctagtggtgtgcttattaatgtggctttgggtaaatgcatttccattgaaaacactacagtcattctggaagactgcgatgggagcaaagagcttcaacaatttaattacacctggttaagacttattaaatgtggagaatggtgtatagcccccatccctgataaaggagccgtaaggctgcacccttgtgataacagaaacaaagggctaaaatggctgcataaatcaacatcagtctttcatccagaactggtgaatcacattgtttttgaaaacaatcagcaattattatgcttggaaggaaatttttctcaaaagatcctgaaagtagctgcctgtgacccagtgaagccatatcaaaagtggaaatttgaaaaatattatgaagcctga

SEQ ID NO: 105 - protein, 168 aa, Homo sapiens; identical to SEQ ID NO: 7

SEQ ID NO: 106 - DNA, 498 bp, Homo sapiens
tccttcggtgacatttcggaacgactgcagctgagggaacaactgcactgtcacaacttttcctggtacctgcacaatgtctacccagagatgtttgttcctgacctgacgcccaccttctatggtgccatcaagaacctcggcaccaaccaatgcctggatgtgggtgagaacaaccgcggggggaagcccctcatcatgtactcctgccacggccttggcggcaaccagtactttgagtacacaactcagagggaccttcgccacaacatcgcaaagcagctgtgtctacatgtcagcaagggtgctctgggccttgggagctgtcacttcactggcaagaatagccaggtccccaaggacgaggaatgggaattggcccaggatcagctcatcaggaactcaggatctggtacctgcctgacatcccaggacaaaaagccagccatggccccctgcaatcccagtgacccccatcagttgtggctctttgtctag

SEQ ID NO: 107 - protein, 165 aa, Homo sapiens; identical to SEQ ID NO: 8

SEQ ID NO: 108 - DNA, 501 bp, Homo sapiens
tatggggatatatcggagctgaaaaaatttcgagaagatcacaactgccaaagttttaagtggttcatggaagaaatagcttatgatatcacctcacactaccctttgccacccaaaaatgttgactggggagaaatcagaggcttcgaaactgcttactgcattgatagcatgggaaaaacaaatggaggctttgttgaactaggaccctgccacaggatgggagggaatcagcttttcagaatcaatgaagcaaatcaactcatgcagtatgaccagtgtttgacaaagggagctgatggatcaaaagttatgattacacactgtaatctaaatgaatttaaggaatggcagtacttcaagaacctgcacagatttactcatattccttcaggaaagtgtttagatcgctcagaggtcctgcatcaagtattcatctccaattgtgactccagtaaaacgactcaaaaatgggaaatgaataacatccatagtgtttag

SEQ ID NO: 109 - protein, 166 aa, Homo sapiens; identical to SEQ ID NO: 9

SEQ ID NO: 110 - DNA, 534 bp, Homo sapiens
gacgtttcttccagaatggcactccgggaaaaactgaaatgtaaaacttttgactggtacctgaaaaatgtttatccactcttgaagccactccacaccatcgtgggctatggaagaatgaaaaacctattggatgaaaatgtctgcttggatcagggacccgttccaggcaacacccccatcatgtattactgccatgaattcagctcacagaatgtctactatcacctaactggggagctctatgtgggacaactgattgcagaggccagtgctagtgatcgctgcctgacagaccctggcaaggcggagaagcccaccttagaaccatgctccaaggcagctaagaatagactgcatatatattgggattttaaaccgggaggagctgtcataaacagagataccaagcggtgtctggagatgaagaaggatcttttgggtagccacgtgcttgtgctccagacctgtagcacgcaagtgtgggaaatccagcacactgtcagagactggggtcagaccaacagccagtga

SEQ ID NO: 111 - protein, 179 aa, Homo sapiens; identical to SEQ ID NO: 10

SEQ ID NO: 112 - DNA, 534 bp, Homo sapiens
ttcggggacgtgtctgagaggctggccctgcgtcagaggctgaagtgtcgcagcttcaagtggtacctggagaacgtgtacccggagatgagggtctacaacaacaccctcacgtacggagaggtgagaaacagcaaagccagtgcctactgtctggaccagggagcggaggacggcgaccgggcgatcctctacccctgccacgggatgtcctcccagctggtgcggtacagcgctgacggcctgctgcagctggggcctctgggctccacagccttcttgcctgactccaagtgtctggtggatgacggcacgggccgcatgcccaccctgaagaggtgtgaggatgtggcgcggccaacacagcggctgtgggacttcacccagagtggccccattgtgagccgggccacgggccgctgcctggaggtggagatgtccaaagatgccaactttgggctccggctggtggtacagaggtgctcggggcagaagtggatgatcagaaactggatcaaacacgcacggcactga

SEQ ID NO: 113 - protein, 177 aa, Homo sapiens; identical to SEQ ID NO: 11

SEQ ID NO: 114 - DNA, 564 bp, Homo sapiens
gctggggatgtcgcagtccagaaaaagctccgcagctcccttaactgcaagagtttcaagtggtttatgacgaagatagcctgggacctgcccaaattctacccacccgtggagcccccggctgcagcttggggggagatccgaaatgtgggcacagggctgtgtgcagacacaaagcacggggccttgggctccccactaaggctagagggctgcgtccgaggccgtggggaggctgcctggaacaacatgcaggtattcaccttcacctggagagaggacatccggcctggagacccccagcacaccaagaagttctgctttgatgccatttcccacaccagccctgtcacgctgtacgactgccacagcatgaagggcaaccagctgtggaaataccgcaaagacaagaccctgtaccaccctgtcagtggcagctgcatggactgcagtgaaagtgaccataggatcttcatgaacacctgcaacccatcctctctcacccagcagtggctgtttgaacacaccaactcaacagtcttggaaaaattcaataggaactga

SEQ ID NO: 115 - protein, 187 aa, Homo sapiens; identical to SEQ ID NO: 12

SEQ ID NO: 116 - DNA, 549 bp, Homo sapiens
tgcaatatcagtgagcgtgtggaactgagaaagaagttgggctgtaaatcatttaaatggtatttggataatgtatacccagagatgcagatatctgggtcccacgccaaaccccaacaacccatttttgtcaatagagggccaaaacgacccaaagtccttcaacgtggaaggctctatcacctccagaccaacaaatgcctggtggcccagggccgcccaagtcagaagggaggtctcgtggtgcttaaggcctgtgactacagtgacccaaatcagatctggatctataatgaagagcatgaattggttttaaatagtctcctttgtctagatatgtcagagactcgctcatcagacccgccacggctcatgaaatgccacgggtcaggaggatcccagcagtggacctttgggaaaaacaatcggctataccaggtgtcggttggacagtgcctgagagcagtggatcccctgggtcagaagggctctgtcgccatggcgatctgcgatggctcctcttcacagcagtggcatttggaaggttaa

SEQ ID NO: 117 - protein, 181 aa, Homo sapiens; identical to SEQ ID NO: 13

SEQ ID NO: 118 - DNA, 525 bp, Homo sapiens
tgggatgtgacagagaggaagcagctccgggacaagctccagtgtaaagacttcaagtggttcttggagactgtgtatccagaactgcatgtgcctgaggacaggcctggcttcttcgggatgctccagaacaaaggactaacagactactgctttgactataaccctcccgatgaaaaccagattgtgggacaccaggtcattctgtacctctgtcatgggatgggccagaatcagtttttcgagtacacgtcccagaaagaaatacgctataacacccaccagcctgagggctgcattgctgtggaagcaggaatggatacccttatcatgcatctctgcgaagaaactgccccagagaatcagaagttcatcttgcaggaggatggatctttatttcacgaacagtccaagaaatgtgtccaggctgcgaggaaggagtcgagtgacagtttcgttccactcttacgagactgcaccaactcggatcatcagaaatggttcttcaaagagcgcatgttatga

SEQ ID NO: 119 - protein, 173 aa, Homo sapiens; identical to SEQ ID NO: 14

SEQ ID NO: 120 - DNA, 528 bp, Homo sapiens
tctgagaagccagactgcatggaacgcttgcagctgcaaaggagactgggttgtcggacattccactggtttctggctaatgtctaccctgagctgtacccatctgaacccaggcccagtttctctggaaagctccacaacactggacttgggctctgtgcagactgccaggcagaaggggacatcctgggctgtcccatggtgttggctccttgcagtgacagccggcagcaacagtacctgcagcacaccagcaggaaggagattcactttggcagcccacagcacctgtgctttgctgtcaggcaggagcaggtgattcttcagaactgcacggaggaaggcctggccatccaccagcagcactgggacttccaggagaatgggatgattgtccacattctttctgggaaatgcatggaagctgtggtgcaagaaaacaataaagatttgtacctgcgtccgtgtgatggaaaagcccgccagcagtggcgttttgaccagatcaatgctgtggatgaacgatga

SEQ ID NO: 121 - protein, 174 aa, Homo sapiens; identical to SEQ ID NO: 15

SEQ ID NO: 122 - DNA, 498 bp, Homo sapiens
tatggagatgtgtcagtcagaaaaacactaagagaaaatctgaagtgtaagcccttttcttggtacctagaaaacatctatccggactcccagatcccaagacgttattactcacttggtgagataagaaatgttgaaaccaatcagtgtttagacaacatgggccgcaaggaaaatgaaaaagtgggtatattcaactgtcatggtatgggaggaaatcaggtattttcttacactgctgacaaagaaatccgaaccgatgacttgtgcttggatgtttctagactcaatggacctgtaatcatgttaaaatgccaccatatgagaggaaatcagttatgggaatatgatgctgagagactcacgttgcgacatgttaacagtaaccaatgtctcgatgaaccttctgaagaagacaaaatggtgcctacaatgcaggactgtagtggaagcagatcccaacagtggctgctaaggaacatgaccttgggcacatga

SEQ ID NO: 123 - protein, 165 aa, Homo sapiens; identical to SEQ ID NO: 16

SEQ ID NO: 124 - DNA, 516 bp, Homo sapiens
tcgggaatgttgagagcagattggacctgaggaagaatctgcgctgccagagcttcaagtggtacctggagaatatctaccctgaactcagcatccccaaggagtcctccatccagaagggcaatatccgacagagacagaagtgcctggaatctcaaaggcagaacaaccaagaaaccccaaacctaaagttgagcccctgtgccaaggtcaaaggcgaagatgcaaagtcccaggtatgggccttcacatacacccagaagatcctccaggaggagctgtgcctgtcagtcatcaccttgttccctggcgccccagtggttcttgtcctttgcaagaatggagatgaccgacagcaatggaccaaaactggttcccacatcgagcacatagcatcccacctctgcctcgatacagatatgttcggtgatggcaccgagaacggcaaggaaatcggcgtcaacccatgtgagtcctcactcatgagccagcactgggacatggtgagttcttgag

SEQ ID NO: 125 - protein, 171 aa, Homo sapiens; identical to SEQ ID NO: 17

SEQ ID NO: 126 - DNA, 492 bp, Homo sapiens
agtgtggctacgcggatagagcagaggaagaagatgaactgcaagtccttccgctggtacctggagaacgtctacccagagctcacggtccccgtgaaggaagcactccccggcatcattaagcagggggtgaactgcttagaatctcagggccagaacacagctggtgacttcctgcttggaatggggatctgcagagggtctgccaagaacccgcagcccgcccaggcatggctgttcagtgaccacctcatccagcagcaggggaagtgcctggctgccacctccaccttaatgtcctcccctggatccccagtcatactgcagatgtgcaaccctagagaaggcaagcagaaatggaggagaaaaggatctttcatccagcattcagtcagtggcctctgcctggagacaaagcctgcccagctggtgaccagcaagtgtcaggctgacgcccaggcccagcagtggcagctgttgccacacacatga

SEQ ID NO: 127 - protein, 163 aa, Homo sapiens; identical to SEQ ID NO: 18